(19) United States
(12) Patent Application Publication
(10) Pub. No.: US 2013/0018033 A1
(43) Pub. Date: Jan. 17, 2013

(54) RAF INHIBITOR COMPOUNDS AND METHODS OF USE THEREOF

(71) Applicants: Ignacio Aliagas, South San Francisco, CA (US); Stefan Gradl, South San Francisco, CA (US); Janet Gunzner, South San Francisco, CA (US); Wendy Lee, South San Francisco, CA (US); Simon Mathieu, South San Francisco, CA (US); Joachim Rudolph, South San Francisco, CA (US); Zhaoyang Wen, South San Francisco, CA (US); Guiling Zhao, South San Francisco, CA (US); Alexandre J. Buckmelter, Boulder, CO (US); Jonas Grina, Boulder, CO (US); Joshua D. Hansen, Boulder, CO (US); Ellen Laird, Boulder, CO (US); David Moreno, Boulder, CO (US); Li Ren, Boulder, CO (US); Steven Mark Wenglowsky, Boulder, CO (US)

(72) Inventors: Ignacio Aliagas, South San Francisco, CA (US); Stefan Gradl, South San Francisco, CA (US); Janet Gunzner, South San Francisco, CA (US); Wendy Lee, South San Francisco, CA (US); Simon Mathieu, South San Francisco, CA (US); Joachim Rudolph, South San Francisco, CA (US); Zhaoyang Wen, South San Francisco, CA (US); Guiling Zhao, South San Francisco, CA (US); Alexandre J. Buckmelter, Boulder, CO (US); Jonas Grina, Boulder, CO (US); Joshua D. Hansen, Boulder, CO (US); Ellen Laird, Boulder, CO (US); David Moreno, Boulder, CO (US); Li Ren, Boulder, CO (US); Steven Mark Wenglowsky, Boulder, CO (US)

(73) Assignee: ARRAY BIOPHARMA INC., Boulder, CO (US)

(21) Appl. No.: 13/393,116

(22) PCT Filed: Aug. 27, 2010
(86) PCT No.: PCT/US2010/046952

§ 371 Date: Feb. 28, 2012

Related U.S. Application Data
(60) Provisional application No. 61/238,105, filed on Aug. 28, 2009.
(60) Provisional application No. 61/312,448, filed on Mar. 10, 2010.

(51) Int. Cl. A61K 31/517; C07D 471/04; A61K 31/519; C07D 405/12; A61P 35/02; C07D 401/12; C07D 413/12; A61K 31/5377; C07D 239/72; A61P 35/00; C07D 239/94; C07D 403/12

(52) U.S. Cl. 514/210.21; 544/293; 514/266.4; 544/279; 514/264.11; 544/119; 514/234.5; 544/284; 544/283; 514/266.1

(57) ABSTRACT

Compounds of Formula I are useful for inhibition of Raf kinases. Methods of using compounds of Formula I and stereoisomers, tautomers, prodrugs and pharmaceutically acceptable salts thereof, for in vitro, in situ, and in vivo diagnosis, prevention or treatment of such disorders in mammalian cells, or associated pathological conditions are disclosed.

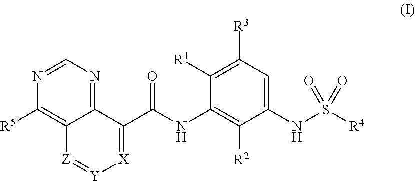

### PRIORITY OF INVENTION

This application claims priority under 35 U.S.C. 119(e) from U.S. Provisional Patent Application No. 61/238,105, filed 28 Aug. 2009 and U.S. Provisional Patent Application No. 61/312,448, filed 10 Mar. 2010, the contents of which are incorporated herein in their entirety.

### BACKGROUND OF THE INVENTION

1. Field of the Invention

The present invention relates to novel compounds, to pharmaceutical compositions comprising the compounds, to a process for making the compounds and to the use of the compounds in therapy. More particularly, it relates to certain substituted compounds useful for inhibiting Raf kinase and for treating disorders mediated thereby.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example RAF-265, GSK-2118436, PLX-4032, PLX-3603 and XL-281. Other B-Raf inhibitors are also known, see for example, U.S. Patent Application Publication 2006/0189627, U.S. Patent Application Publication 2006/0281751, U.S. Patent Application Publication 2007/0049603, U.S. Patent Application Publication 2009/0176809, International Patent Application Publication WO 2007/002325, International Patent Application Publication WO 2007/002433, International Patent Application Publication WO 2008/028141, International Patent Application Publication WO 2008/079903, International Patent Application Publication WO 2008/079906 and International Patent Application Publication WO 2009/012283. International Patent Application Publication WO 2006/066913, International Patent Application Publication WO 2008/028617 and International Patent Application Publication WO 2008/079909 also disclose kinase inhibitors.

### SUMMARY OF THE INVENTION

In one aspect, the invention relates to compounds that are inhibitors of Raf kinases, particularly B-Raf inhibitors. Certain hyperproliferative disorders are characterized by the overactivation of Raf kinase function, for example by mutations or overexpression of the protein. Accordingly, the compounds of the invention are useful in the treatment of hyperproliferative disorders, such as cancer.

More specifically, one aspect of the present invention provides compounds of Formula I:

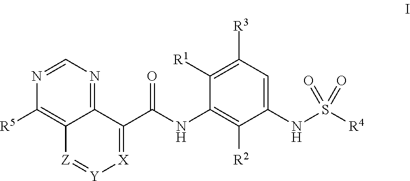

and stereoisomers, tautomers and pharmaceutically acceptable salts thereof, wherein R¹, R², R³, R⁴ and R⁵ are as defined herein.

Another aspect of the present invention provides methods of preventing or treating a disease or disorder modulated by B-Raf, comprising administering to a mammal in need of such treatment an effective amount of a compound of this invention or a stereoisomer, tautomer, prodrug or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating a disease or disorder modulated by B-Raf, comprising administering to a mammal in need of such treatment an effective amount of a compound of this invention or a stereoisomer, tautomer, or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating cancer, comprising administering to a mammal in need of such treatment an effective amount of a compound of this invention, or a stereoisomer, tautomer, prodrug or pharmaceutically acceptable salt thereof, alone or in combination with one or more additional compounds having anti-cancer properties.

Another aspect of the present invention provides methods of preventing or treating cancer, comprising administering to a mammal in need of such treatment an effective amount of a compound of this invention, or a stereoisomer, tautomer or pharmaceutically acceptable salt thereof, alone or in combination with one or more additional compounds having anti-cancer properties.

Another aspect of the present invention provides a method of treating a hyperproliferative disease in a mammal comprising administering a therapeutically effective amount of a compound of this invention to the mammal.

Another aspect of the present invention provides methods of preventing or treating kidney disease, comprising administering to a mammal in need of such treatment an effective amount of a compound of this invention, or a stereoisomer, tautomer, prodrug or pharmaceutically acceptable salt thereof, alone or in combination with one or more additional compounds. Another aspect of the present invention provides methods of preventing or treating polycystic kidney disease, comprising administering to a mammal in need of such treatment an effective amount of a compound of this invention, or a stereoisomer, tautomer, prodrug or pharmaceutically acceptable salt thereof, alone or in combination with one or more additional compounds.

Another aspect of the present invention provides the compounds of the present invention for use in therapy.

Another aspect of the present invention provides the compounds of the present invention for use in the treatment of a hyperproliferative disease. In a further embodiment, the hyperproliferative disease may be cancer (or still further, a specific cancer as defined herein).

Another aspect of the present invention provides the compounds of the present invention for use in the treatment of a kidney disease. In a further embodiment, the kidney disease may be polycystic kidney disease.

Another aspect of the present invention provides the use of a compound of this invention in the manufacture of a medicament for the treatment of a hyperproliferative disease. In a further embodiment, the hyperproliferative disease may be cancer (or still further, a specific cancer as defined herein).

Another aspect of the present invention provides the use of a compound of this invention in the manufacture of a medicament for the treatment of a kidney disease. In a further embodiment, the kidney disease may be polycystic kidney disease.

Another aspect of the present invention provides the use of a compound of the present invention in the manufacture of a medicament, for use as a B-Raf inhibitor in the treatment of a patient undergoing cancer therapy.

Another aspect of the present invention provides the use of a compound of the present invention in the manufacture of a medicament, for use as a B-Raf inhibitor in the treatment of a patient undergoing polycystic kidney disease therapy.

Another aspect of the present invention provides a pharmaceutical composition comprising a compound of the present invention for use in the treatment of a hyperproliferative disease.

Another aspect of the present invention provides a pharmaceutical composition comprising a compound of the present invention for use in the treatment of cancer.

Another aspect of the present invention provides a pharmaceutical composition comprising a compound of the present invention for use in the treatment of polycystic kidney disease.

Another aspect of the present invention provides a pharmaceutical composition comprising a compound of this invention, a stereoisomer, tautomer, prodrug or a pharmaceutically acceptable salt thereof, and a pharmaceutically acceptable carrier or excipient.

Another aspect of the present invention provides a pharmaceutical composition comprising a compound of this invention or a pharmaceutically acceptable salt thereof, and a pharmaceutically acceptable carrier or excipient.

Another aspect of the present invention provides intermediates for preparing compounds of Formulas I-VIII. Certain compounds of Formulas I-VIII may be used as intermediates for other compounds of Formulas I-VIII.

Another aspect of the present invention includes methods of preparing, methods of separation, and methods of purification of the compounds of this invention.

### BRIEF DESCRIPTION OF THE FIGURES

FIG. 1 shows a TGI experiment in nude mice with subcutaneous LOX xenografts.

FIG. 2 shows a TGI experiment in nude mice with subcutaneous LOX xenografts.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” includes linear or branched-chain radicals of carbon atoms. In one example, the alkyl radical is one to six carbon atoms (C₁-C₆). In other examples, the alkyl radical is C₁-C₅, C₁-C₄ or C₁-C₃. C₀ refers to a bond. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. Other examples of alkyl groups include 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂) and 3,3-dimethyl-2-butyl (—CH(CH₃)C(CH₃)₃. The abbreviations are sometimes used in conjunction with elemental abbreviations and chemical structures, for example, methanol (“MeOH”) or ethanol (“EtOH”).

Additional abbreviations used throughout the application include, for example, benzyl (“Bn”), phenyl (“Ph”) and acetyl (“Ac”).

The following terms are abbreviated: ethylacetate (“EtOAc”), dimethylsulfoxide (“DMSO”), dimethylformamide (“DMF”), dichloromethane (“DCM”) and tetrahydrofuran (“THF”).

The term “alkenyl” refers to linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to six carbon atoms (C₂-C₆). In other examples, the alkenyl radical is C₂-C₅, C₂-C₄ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, hexa-1,3-dienyl.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to six carbon atoms (C₂-C₆). In other examples, the alkynyl radical is C₂-C₅, C₂-C₄ or C₂-C₃. Examples include, but are not limited to, ethynyl prop-1-ynyl (—C≡CCH₃), prop-2-ynyl (propargyl, CH₂C≡CH), but-1-ynyl, but-2-ynyl and but-3-ynyl.

The term “alkoxy” refers to a linear or branched monovalent radical represented by the formula —OR in which R is alkyl, alkenyl, alkynyl or cycloalkyl, which can be further optionally substituted as defined herein. Alkoxy groups include methoxy, ethoxy, 2-methoxyethoxy, propoxy, isopropoxy, mono-, di- and tri-fluoromethoxy and cyclopropoxy.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 6 carbon atoms (C₃-C₆). In other examples, cycloalkyl is C₃-C₄ or C₃-C₅. In other examples, the cycloalkyl group, as a monocycle, is C₃-C₆ or C₅-C₆. In another example, the cycloalkyl group, as a bicycle, is C₇-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane, and bicyclo[3.2.2]nonane.

The terms “heterocyclic” or “heterocycle” or “heterocyclyl” refers to a saturated or a partially unsaturated (i.e., having one or more double and/or triple bonds within the ring) cyclic group in which at least one ring atom is a heteroatom independently selected from nitrogen, oxygen, and sulfur, the remaining ring atoms being carbon. In one embodiment, heterocyclyl includes saturated or partially unsaturated 4-6 membered heterocyclyl groups, another embodiment includes 5-6 membered heterocyclyl groups. The heterocyclyl group may be optionally substituted with one or more substituents described herein. Exemplary heterocyclyl groups include, but are not limited to, oxiranyl, aziridinyl, thiiranyl, azetidinyl, oxetanyl, thietanyl, 1,2-dithietanyl, 1,3-dithietanyl, pyrrolidinyl, piperidinyl, dihydropyridinyl, tetrahydropyridinyl, morpholinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, imidazolidinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Heterocycles include 4 to 6 membered rings containing one or two heteroatoms selected from oxygen, nitrogen and sulfur.

The term “heteroaryl” refers to an aromatic cyclic group in which at least one ring atom is a heteroatom independently selected from nitrogen, oxygen and sulfur, the remaining ring atoms being carbon. Heteroaryl groups may be optionally substituted with one or more substituents described herein. In one example, heteroaryl includes 5-6 membered heteroaryl groups. Other examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, and furopyridinyl. Heteroaryls includes 5 to 6 membered aromatic rings containing one, two or three heteroatoms selected from oxygen, nitrogen and sulfur.

“Halogen” refers to F, Cl, Br or I.

The abbreviation “TLC” stands for thin layer chromatography.

The terms “treat” or “treatment” refer to therapeutic, prophylactic, palliative or preventative measures. In one example, treatment includes therapeutic and palliative treatment. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound of the present invention that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term cancer may be used generically to include various types of cancer or specifically (as listed above).

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound of the invention.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

The term “mammal” means a warm-blooded animal that has or is at risk of developing a disease described herein and includes, but is not limited to, guinea pigs, dogs, cats, rats, mice, hamsters, and primates, including humans.

The terms “compound of this invention,” “compounds of the present invention” and “compounds of Formula I,” unless otherwise indicated, include compounds of Formulas I, II, III, IV, V, VI, VII and/or VIII, stereoisomers, tautomers, solvates, metabolites, salts (e.g., pharmaceutically acceptable salts) and prodrugs thereof. Unless otherwise stated, structures depicted herein are also meant to include compounds that differ only in the presence of one or more isotopically enriched atoms. For example, compounds of Formulas I, II, III, IV, V, VI, VII and/or VIII, wherein one or more hydrogen atoms are replaced deuterium or tritium, or one or more carbon atoms are replaced by a ¹³C- or ¹⁴C-enriched carbon are within the scope of this invention.

B-RAF Inhibitor Compounds

The present invention provides compounds, and pharmaceutical formulations thereof, that are potentially useful in the treatment of diseases, conditions and/or disorders modulated by B-Raf.

One embodiment of this invention provides compounds of Formula I:

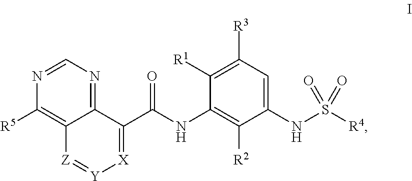

stereoisomers, tautomers, prodrugs and pharmaceutically acceptable salts thereof, wherein:

X is N or CR¹²;

Y is N or CR¹³;

Z is N or CR¹⁴, wherein no more than two of X, Y and Z can be N at the same time;

R¹ and R² are independently selected from hydrogen, halogen, —CN, —C(O)NR⁶R⁷, C₁-C₃ alkyl, C₂-C₃ alkenyl, C₂-C₃ alkynyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, 3-6 membered heterocyclyl, a 5-6 membered heteroaryl or NR⁶R⁷, wherein the cycloalkyl, alkyl, alkenyl, alkynyl, phenyl, heterocyclyl and heteroaryl are optionally substituted with OR¹⁵, halogen, phenyl, C₃-C₄ cycloalkyl or C₁-C₄ alkyl optionally substituted with halogen;

R⁵ is hydrogen, C₁-C₆ alkyl, or NR⁸R⁹;

R⁶ and R⁷ are each independently hydrogen or C₁-C₆ alkyl optionally substituted by halogen; or

R⁶ and R⁷ are independently taken together with the atom to which they are attached to form a 3-6 membered heterocyclyl optionally substituted by halogen, oxo or C₁-C₃ alkyl;

R⁸ is hydrogen;

R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)₃₋₆-membered heterocyclyl or (C₀-C₃ alkyl)₅₋₆-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;

R¹⁰ and R¹¹ are independently hydrogen or C₁-C₆ alkyl optionally substituted by halogen; or

R¹⁰ and R¹¹ are taken together with the atom to which they are attached to form a 3-6 membered heterocyclyl optionally substituted by halogen, oxo or C₁-C₃ alkyl;

R¹² is hydrogen, C₁-C₃ alkyl or halogen;

R¹³ is hydrogen, C₂-C₆ alkyl, C₂-C₆ alkenyl, C₂-C₆ alkynyl or halogen, wherein said alkyl, alkenyl and alkynyl are optionally substituted by OR¹⁸;

R¹⁴ is hydrogen, C₁-C₃ alkyl or halogen;

R¹⁵ is hydrogen or C₁-C₃ alkyl optionally substituted by halogen;

R¹⁶ and R¹⁷ are independently hydrogen or C₁-C₆ alkyl optionally substituted by halogen; or

R¹⁶ and R¹⁷ are taken together with the atom to which they attached to form a 3-6 membered heterocyclyl optionally substituted by halogen, oxo or C₁-C₃ alkyl; and

R¹⁸ is hydrogen or C₁-C₃ alkyl.

One embodiment includes compounds of Formula I, stereoisomers, tautomers, prodrugs and pharmaceutically acceptable salts thereof, wherein:

X is N or CR¹²;

Y is N or CR¹³;

Z is N or CR¹⁴, wherein no more than two of X, Y and Z can be N at the same time;

R¹ and R² are independently selected from hydrogen, halogen, CN, C₁-C₃ alkyl, C₂-C₃ alkenyl, C₂-C₃ alkynyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl or NR⁶R⁷, wherein the cycloalkyl, alkyl, alkenyl, alkynyl, phenyl and heteroaryl are optionally substituted with OR¹⁵, halogen, phenyl, C₃-C₄ cycloalkyl or C₁-C₄ alkyl optionally substituted with halogen;

R⁵ is hydrogen or NR⁸R⁹;

R⁶ and R⁷ are each independently hydrogen or C₁-C₆ alkyl optionally substituted by halogen; or

R⁶ and R⁷ are independently taken together with the atom to which they are attached to form a 3-6 membered heterocyclyl optionally substituted by halogen, oxo or C₁-C₃ alkyl;

R⁸ is hydrogen;

R⁹ is hydrogen, (C₀-C₃alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)3-6-membered heterocyclyl or (C₀-C₃ alkyl)5-6-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;

R¹⁰ and R¹¹ are independently hydrogen or C₁-C₆ alkyl optionally substituted by halogen; or

R¹⁰ and R¹¹ are taken together with the atom to which they are attached to form a 3-6 membered heterocyclyl optionally substituted by halogen, oxo or C₁-C₃ alkyl;

R¹² is hydrogen, C₁-C₃ alkyl or halogen;

R¹³ is hydrogen, C₁-C₃ alkyl, C₂-C₃ alkenyl, C₂-C₃ alkynyl or halogen, wherein said alkyl, alkenyl and alkynyl are optionally substituted by OR¹⁸;

R¹⁴ is hydrogen, C₁-C₃ alkyl or halogen;

R¹⁵ is hydrogen or C₁-C₃ alkyl optionally substituted by halogen;

R¹⁶

and R¹⁷ are independently hydrogen or C₁-C₆ alkyl optionally substituted by halogen; or

R¹⁶ and R¹⁷ are taken together with the atom to which they attached to form a 3-6 membered heterocyclyl optionally substituted by halogen, oxo or C₁-C₃ alkyl; and

R¹⁸ is hydrogen or C₁-C₃ alkyl.

One embodiment of this invention provides compounds of Formula I:

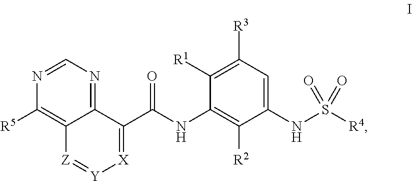

stereoisomers, tautomers, prodrugs and pharmaceutically acceptable salts thereof, wherein:

X is N or CR¹²;

Y is N or CR¹³;

Z is N or CR¹⁴, wherein no more than two of X, Y and Z can be N at the same time;

R¹ and R² are independently selected from hydrogen, halogen, CN, C₁-C₃ alkyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl or NR⁶R⁷, wherein the cycloalkyl, alkyl, alkenyl, alkynyl, phenyl and heteroaryl are optionally substituted with OR¹⁵, halogen, phenyl, C₃-C₄ cycloalkyl or C₁-C₄ alkyl optionally substituted with halogen;

R⁵ is hydrogen or NR⁸R⁹;

R⁶ and R⁷ are each independently hydrogen or C₁-C₆ alkyl optionally substituted by halogen; or

R⁶ and R⁷ are independently taken together with the atom to which they are attached to form a 3-6 membered heterocyclyl optionally substituted by halogen, oxo or C₁-C₃ alkyl;

R⁸ is hydrogen;

R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)3-6-membered heterocyclyl or (C₀-C₃ alkyl)5-6-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;

R¹⁰ and R¹¹ are independently hydrogen or C₁-C₆ alkyl optionally substituted by halogen; or

R¹⁰ and R¹¹ are taken together with the atom to which they are attached to form a 3-6 membered heterocyclyl optionally substituted by halogen, oxo or C₁-C₃ alkyl;

R¹² is hydrogen, C₁-C₃ alkyl or halogen;

R¹³ is hydrogen, C₁-C₃ alkyl or halogen;

R¹⁴ is hydrogen, C₁-C₃ alkyl or halogen;

R¹⁵ is hydrogen or C₁-C₃ alkyl optionally substituted by halogen; and

R¹⁶ and R¹⁷ are independently hydrogen or C₁-C₆ alkyl optionally substituted by halogen; or

R¹⁶ and R¹⁷ are taken together with the atom to which they attached to form a 3-6 membered heterocyclyl optionally substituted by halogen, oxo or C₁-C₃ alkyl.

One embodiment of this invention provides compounds of Formula I, stereoisomers, tautomers and pharmaceutically acceptable salts thereof.

In certain embodiments, X is CR¹², Y is N and Z is CR¹⁴. In certain embodiments, X is CH, Y is N and Z is CH.

In certain embodiments, X is CR¹², Y is CR¹³ and Z is N. In certain embodiments, X is CH, Y is CH and Z is N.

In certain embodiments, X is CR¹², Y is CR¹³ and Z is CR¹⁴. In certain embodiments, X is CH, Y is CH and Z is CH. In certain embodiments, X is CH, Y is CR¹³ and Z is CH.

In certain embodiments, R¹ and R² are independently selected from hydrogen, halogen, CN, C₁-C₃ alkyl, C₁-C₃ alkynyl or C₁-C₃ alkoxy.

In certain embodiments, R¹ and R² are independently selected from hydrogen, halogen, CN, C₁-C₃ alkyl or C₁-C₃ alkoxy.

In certain embodiments, R¹, R² and R³ are independently selected from hydrogen, halogen or C₁-C₃ alkyl.

In certain embodiments, R¹, R² and R³ are independently selected from hydrogen, F, Cl or methyl.

In certain embodiments, R¹ and R³ are independently selected from hydrogen, halogen or C₁-C₃ alkyl, and R² is Cl. In certain embodiments, R¹ and R³ are independently selected from hydrogen, F, Cl and methyl, and R² is Cl.

In certain embodiments, R¹ is hydrogen, halogen, CN, C₁-C₃ alkyl or C₁-C₃ alkoxy.

In certain embodiments, R¹ is hydrogen.

In certain embodiments, R¹ is halogen. In certain embodiments, R¹ is F or Cl.

In certain embodiments, R¹ is C₁-C₃ alkyl. In certain embodiments, R¹ is methyl.

In certain embodiments, R² is hydrogen, halogen, CN, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl. In certain embodiments, R² is Cl.

In certain embodiments, R² is hydrogen.

In certain embodiments, R³ is hydrogen, halogen or C₁-C₃ alkyl.

In certain embodiments, R³ is hydrogen.

In certain embodiments, R³ is halogen. In certain embodiments, R³ is F or Cl.

In certain embodiments, R¹ and R² are F and R³ is hydrogen.

In certain embodiments, R¹ is F and R² is Cl and R³ is hydrogen.

In certain embodiments, R¹ is Cl and R² is F and R³ is hydrogen.

In certain embodiments, R¹ is F and R² and R³ are hydrogen.

In certain embodiments, R¹ and R³ are hydrogen and R² is F.

In certain embodiments, R¹ and R³ are hydrogen and R² is Cl.

In certain embodiments, R² and R³ are F and R¹ is hydrogen.

In certain embodiments, R¹ is Cl and R² and R³ are hydrogen.

In certain embodiments, R¹ is methyl and R² and R³ are hydrogen.

In certain embodiments, R¹, R² and R³ are F.

In certain embodiments, R¹ is F and R² is methyl and R³ is hydrogen.

In certain embodiments, R¹ is methyl and R² is F and R³ is hydrogen.

In certain embodiments, R¹ is F and R² and R³ are hydrogen.

In certain embodiments, R¹ is Cl and R² and R³ are hydrogen.

In certain embodiments, R² is F and R¹ and R³ are hydrogen.

In certain embodiments, R¹ is hydrogen and R² and R³ are F.

In certain embodiments, R¹ is hydrogen, R² is F and R³ is Cl.

In certain embodiments, R¹ and R³ are hydrogen and R² is —CN.

In certain embodiments, R¹ is F, R² is —CN and R³ is hydrogen.

In certain embodiments, R¹ is Cl, R² is —CN and R³ is hydrogen.

In certain embodiments, R¹ and R² are Cl and R³ is hydrogen.

In certain embodiments, R¹ is F, R² is methoxy and R³ is hydrogen.

In certain embodiments, R¹ is Cl, R² is ethynyl and R³ is hydrogen.

In certain embodiments, R¹ is —C(O)NR⁶R⁷. In certain embodiments, R¹ is —C(O)NH₂.

In certain embodiments, the residue:

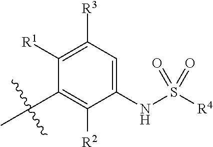

of Formula I, wherein the wavy line represents the point of attachment of the residue in Formula I, is selected from:

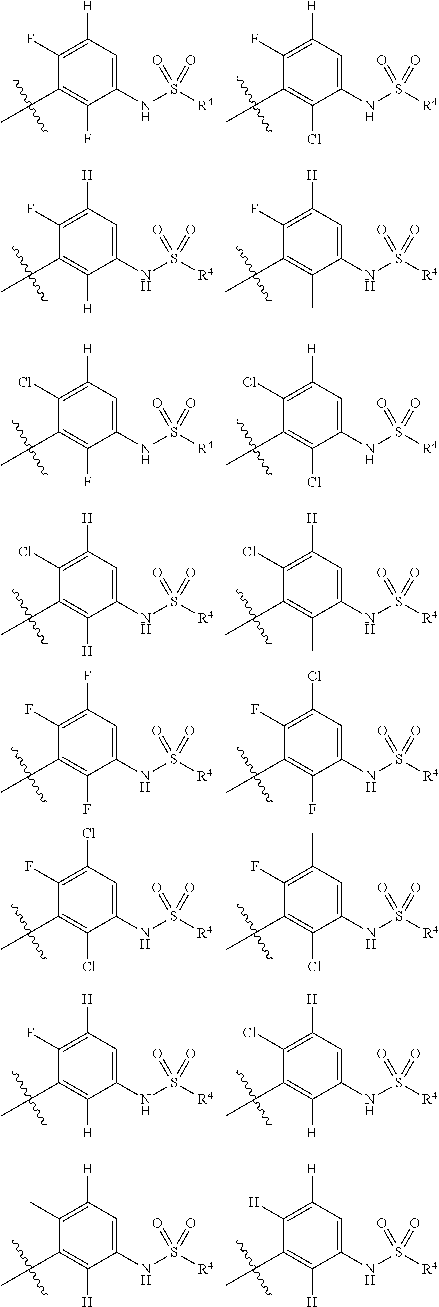

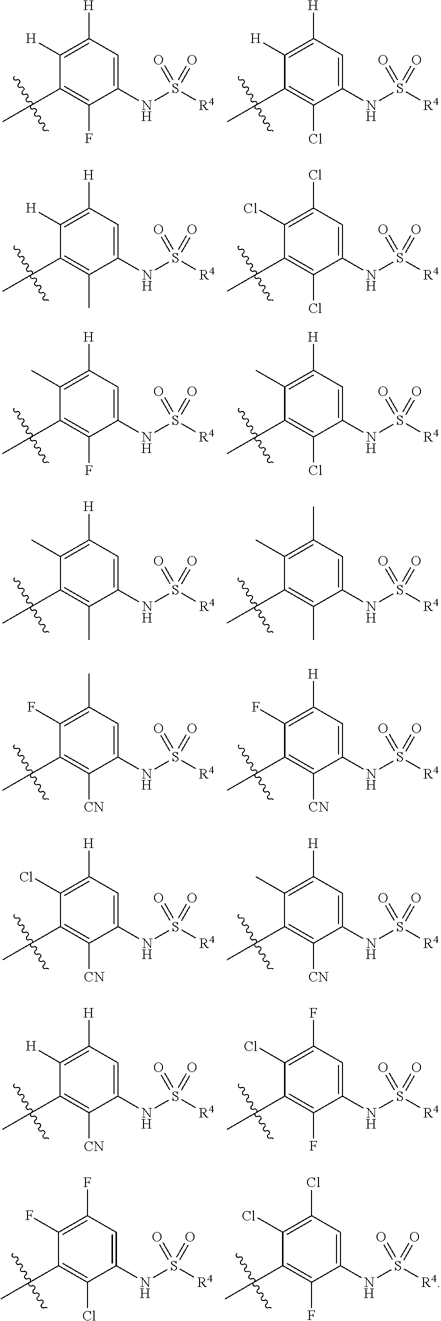

In certain embodiments, the residue:

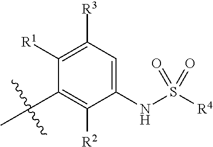

of Formula I, wherein the wavy line represents the point of attachment of the residue in Formula I, is selected from:

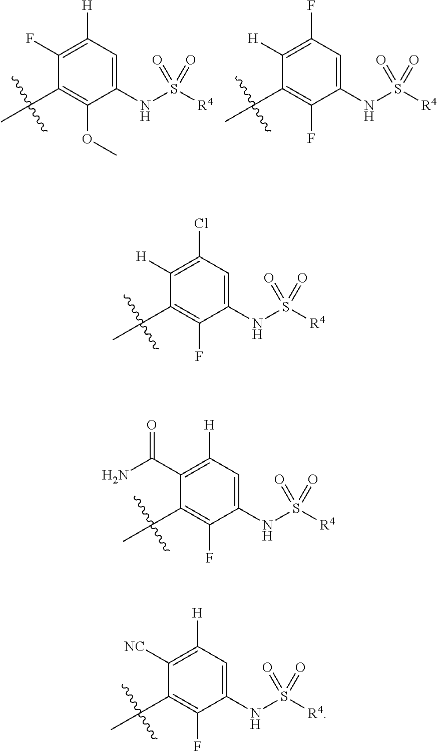

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl or NR⁶R⁷, wherein the cycloalkyl, alkyl, alkenyl, alkynyl, phenyl and heteroaryl are optionally substituted with OR¹⁵, halogen, phenyl, C₃-C₄ cycloalkyl, or C₁-C₄ alkyl optionally substituted with halogen.

In certain embodiments, R⁴ is C₃-C₄ cycloalkyl, C₁-C₆ alkyl optionally substituted with halogen or C₃-C₄ cycloalkyl, or NR⁶R⁷. In certain embodiments, R⁶ and R⁷ are independently selected from hydrogen and C₁-C₅ alkyl.

In certain embodiments, R⁴ is C₃-C₅ cycloalkyl, C₁-C₆ alkyl, C₂-C₆ alkenyl or C₂-C₆ alkynyl, wherein the cycloalkyl, alkyl, alkenyl and alkynyl are optionally substituted with OR¹⁵, halogen or C₃-C₄ cycloalkyl.

In certain embodiments, R⁴ is cyclopropyl, ethyl, propyl, butyl, isobutyl, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —N(CH₃)₂, or pyrrolidinyl.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl, —NHCH₂CH₂CH₃, —N(CH₃)CH₂CH₃, —N(CH₃)₂, or pyrrolidine.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl or —NHCH₂CH₂CH₃.

In certain embodiments, R⁴ is propyl, butyl, isobutyl, —CH₂CH₂CH₂F, —CH₂CH₂CF₃ or cyclopropylmethyl.

In certain embodiments, R⁴ is C₃-C₅ cycloalkyl or C₁-C₆ alkyl optionally substituted with OH, halogen or C₃-C₄ cycloalkyl.

In certain embodiments, R⁴ is C₃-C₅ cycloalkyl. In certain embodiments, R⁴ is C₃-C₄ cycloalkyl. In certain embodiments, R⁴ is cyclopropyl or cyclobutyl.

In certain embodiments, R⁴ is C₁-C₆ alkyl. In certain embodiments, R⁴ is ethyl, propyl, butyl or isobutyl. In certain embodiments, R⁴ is propyl.

In certain embodiments, R⁴ is C₁-C₆ alkyl optionally substituted with halogen. In certain embodiments, R⁴ is —CF₃, —CH₂Cl, —CH₂CF₃, —CH₂CH₂CH₂F, —CH₂CH₂CF₃, —CF₂CF₃ or —CF₂CF₂CF₃.

In certain embodiments, R⁴ is C₁-C₆ alkyl optionally substituted with OH, halogen or C₃-C₄ cycloalkyl. In certain embodiments, R⁴ is cyclopropylmethyl (—CH₂-cyclopropyl) or cyclobutylmethyl (—CH₂-cyclobutyl). In certain embodiments, R⁴ is cyclopropylmethyl (—CH₂-cyclopropyl).

In certain embodiments, R⁴ is C₁-C₆ alkyl optionally substituted with phenyl. In certain embodiments, R⁴ is phenylmethyl.

In certain embodiments, R⁴ is phenyl optionally substituted with OR¹⁵, halogen, C₃-C₄ cycloalkyl, or C₁-C₄ alkyl optionally substituted with halogen. In certain embodiments, R⁴ is phenyl optionally substituted with halogen. In certain embodiments, R⁴ is phenyl optionally substituted with C₁-C₄ alkyl optionally substituted with halogen. In certain embodiments, R⁴ is phenyl optionally substituted with halogen and C₁-C₄ alkyl optionally substituted with halogen. In certain embodiments, R⁴ is phenyl. In certain embodiments, R⁴ is phenyl, 2-fluorophenyl, 3-fluorophenyl, 4-fluorophenyl, 2,5-difluorophenyl or 4-chloro-3-trifluoromethylphenyl.

In certain embodiments, R⁴ is a 5-6 membered heteroaryl optionally substituted with OR¹⁵, halogen, C₃-C₄ cycloalkyl or C₁-C₄ alkyl optionally substituted with halogen. In certain embodiments, R⁴ is a 5-6 membered heteroaryl optionally substituted with C₁-C₄ alkyl. In certain embodiments, R⁴ is a 5-6 membered heteroaryl, wherein the heteroaryl contains one or two heteroatoms selected from the group consisting of oxygen, nitrogen and sulfur. In certain embodiments, R⁴ is a 5-6 membered heteroaryl, wherein the heteroaryl is imidazolyl, furanyl, pyridinyl or thiophenyl. In certain embodiments, R⁴ is 1-methyl-1H-imidazol-4-yl, furan-2-yl, pyridin-2-yl, pyridin-3-yl or thiophen-2-yl.

In certain embodiments, R⁴ is NR⁶R⁷. In certain embodiments, R⁶ and R⁷ are independently selected from hydrogen and C₁-C₆ alkyl. In certain embodiments, R⁶ is hydrogen. In certain embodiments, R⁶ is C₁-C₆ alkyl. In certain embodiments, R⁶ is methyl, ethyl or propyl. In certain embodiments, R⁷ is hydrogen or methyl. In certain embodiments, R⁴ is selected from the group consisting of —NHCH₂CH₃, —NHCH₂CH₂CH₃, —N(CH₃)CH₂CH₃ and —N(CH₃)₂.

In certain embodiments, R⁶ and R⁷ together with the nitrogen to which they are attached form a 4 to 6 membered heterocyclic ring. In certain embodiments, R⁶ and R⁷ together with the nitrogen to which they are attached form a 4 to 6 membered heterocyclic ring, wherein the heterocyclic ring contains one nitrogen heteroatom. In certain embodiments, R⁴ is pyrrolidine.

In certain embodiments, R⁴ is selected from propyl, cyclopropylmethyl, —CH₂CH₂CH₂F and phenyl. In a further embodiment, R⁴ is selected from propyl, cyclopropylmethyl and —CH₂CH₂CH₂F.

An embodiment of Formula I provides compounds of Formulas II-V:

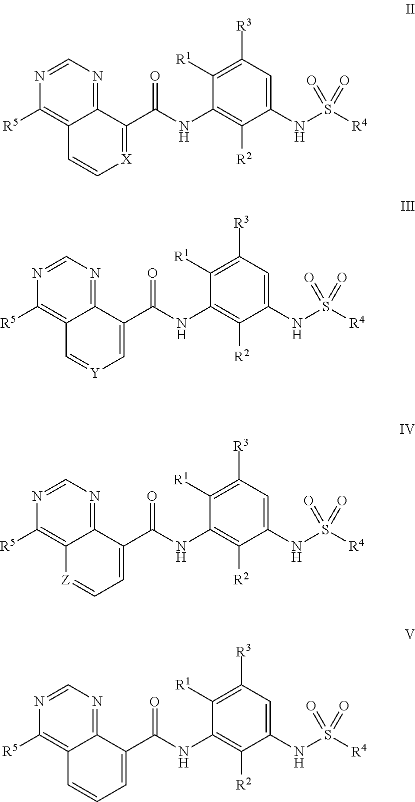

In certain embodiments of Formula I, R¹ and R² are F, R³ is hydrogen and R⁴ is propyl, such that the compounds have the structure of Formula VI:

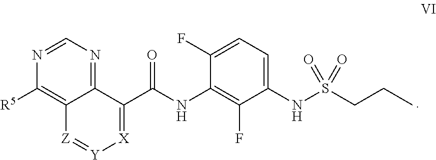

In certain embodiments of Formula I, R¹ is Cl and R² is F, R³ is hydrogen and R⁴ is propyl, such that the compounds have the structure of Formula VII:

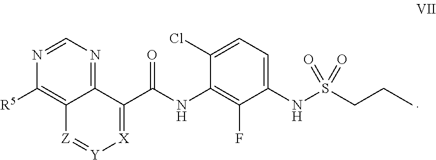

In certain embodiments of Formula I, R¹ is F and R² is Cl, R³ is hydrogen and R⁴ is propyl, such that the compounds have the structure of Formula VIII:

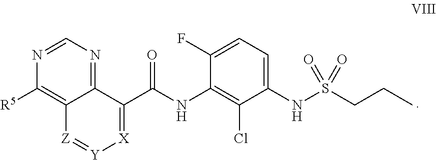

In certain embodiments, R⁵ is hydrogen.

In certain embodiments, R⁵ is NR⁸R⁹, R⁸ is hydrogen and R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)₃₋₆-membered heterocyclyl or (C₀-C₃ alkyl)₅₋₆-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl.

In certain embodiments, R⁵ is NR⁸R⁹, and R⁸ and R⁹ are hydrogen.

In certain embodiments, R⁵ is NR⁸R⁹, R⁸ is hydrogen and R⁹ is hydrogen or C₁-C₃ alkyl.

In certain embodiments, R⁵ is NR⁸R⁹, R⁸ is hydrogen and R⁹ is C₁-C₃ alkyl optionally substituted by halogen. In certain embodiments, R⁹ is 2-fluoroethyl.

In certain embodiments, R⁵ is NR⁸R⁹, R⁸ is hydrogen and R⁹ is C₃-C₆ cycloalkyl optionally substituted by halogen. In certain embodiments, R⁹ is cyclopropyl, cyclobutyl, cyclopentyl, cyclohexyl or 4,4-difluorocyclohexyl.

In certain embodiments, R⁵ is NR⁸R⁹, R⁸ is hydrogen and R⁹ is 3-6-membered heterocyclyl optionally substituted by C₁-C₃ alkyl. In certain embodiments, R⁹ is N-methylazetidinyl, morpholinyl, tetrahydropyranyl or piperidinyl.

In certain embodiments, R¹² is hydrogen.

In certain embodiments, R¹³ is hydrogen.

In certain embodiments, R¹³ is C₁-C₆ alkyl. In certain embodiments, R¹³ is methyl.

In certain embodiments, X is CH, Z is CH, Y is CR¹³, and R¹³ is methyl.

In certain embodiments, R¹³ is halogen. In certain embodiments, R¹³ is F.

In certain embodiments, X is CH, Z is CH, Y is CR¹³, and R¹³ is F.

In certain embodiments, R¹³ is C₂-C₆ alkynyl optionally substituted by OR¹⁸. In certain embodiments, R¹³ is —C≡CCH₂OH.

In certain embodiments, R¹⁴ is hydrogen.

In certain embodiments of Formula I, R¹ is halogen, R² is halogen or OCH₃, R³ is hydrogen, R⁴ is C₁-C₃ alkyl, R⁵ is hydrogen or NH₂, X and Z are CH, and Y is CH or N.

In certain embodiments, X is CH, Z is CH, Y is CR¹³, R¹³ is methyl, R⁵ is NR⁸R⁹, R⁸ is hydrogen and R⁹ is C₃-C₆ cycloalkyl optionally substituted by halogen.

In certain embodiments, X, Y and Z are CH; R¹ and R² are Cl or F; R³ is hydrogen; R⁴ is C₃-C₄ alkyl optionally substituted by halogen; and R⁵ is NH₂.

It will be appreciated that certain compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

It will also be appreciated that compounds of Formulas I-VIII include tautomeric forms. Tautomers are compounds that are interconvertible by tautomerization. This commonly occurs due to the migration of a hydrogen atom or proton, accompanied by the switch of a single bond and adjacent double bond. Tautomers of Formulas I-VIII may form at positions, including, but not limited to, the sulfonamide or R⁵ position depending on the substitution. The compounds of Formulas I-VIII are intended to include all tautomeric forms.

It will also be appreciated that certain compounds of Formulas I-VIII may be used as intermediates for further compounds of Formulas I-VIII.

It will be further appreciated that the compounds of the present invention may exist in unsolvated, as well as solvated forms with pharmaceutically acceptable solvents, such as water, ethanol, and the like, and it is intended that the invention embrace both solvated and unsolvated forms.

The term “prodrug” as used in this application refers to a precursor or derivative form of a compound of the invention that is less active or inactive compared to the parent compound or drug and is capable of being metabolized in vivo into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, N-methyl prodrugs (including N-methyl sulfonamide prodrugs), phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug.

Prodrugs of compounds of Formulas I-VIII may not be as active as the compounds of Formulas I-VIII in the assay as described in Example A. However, the prodrugs are capable of being converted in vivo into more active metabolites of compounds of Formulas

Synthesis of Compounds

Compounds of the present invention may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

In preparing compounds of Formulas I-VIII, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”), p-methoxybenzyl (“PMB”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

For illustrative purposes, Schemes 1-26 show general methods for preparing the compounds of the present invention, as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

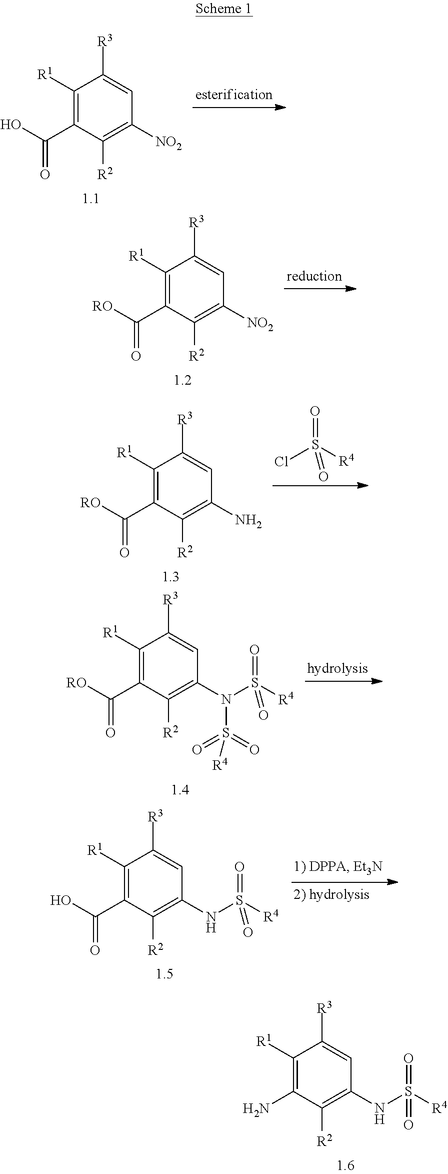

Scheme 1 shows a general method for preparing a compound 1.6, wherein R¹, R², R³ and R⁴ are as defined herein. A benzoic acid 1.1 is esterified to an alkyl benzoate 1.2 (where R is alkyl), e.g. by treatment with trimethylsilyl diazomethane in MeOH, or via Fischer esterification conditions, such as treatment with trimethylsilyl chloride (“TMSCl”) in MeOH. Reduction of nitro intermediate 1.2 to its amino analog 1.3 is performed using a standard condition, such as treatment with Pd/C and H₂. Bis-sulfonamide 1.4 is obtained by treatment of the aniline 1.3 with a sulfonyl chloride R⁴SO₂Cl in the presence of a base, such as NEt₃, in an organic solvent, such as dichloromethane (“DCM”). Hydrolysis of compound 1.4 is accomplished under basic conditions, such as aqueous NaOH, in the appropriate solvent system, such as THF and/or MeOH, to provide a carboxylic acid 1.5. This compound in a suitable solvent, such as THF, is treated with diphenylphosphonic azide (“DPPA”) and a base such as triethylamine, and subsequently hydrolyzed to form an amine 1.6.

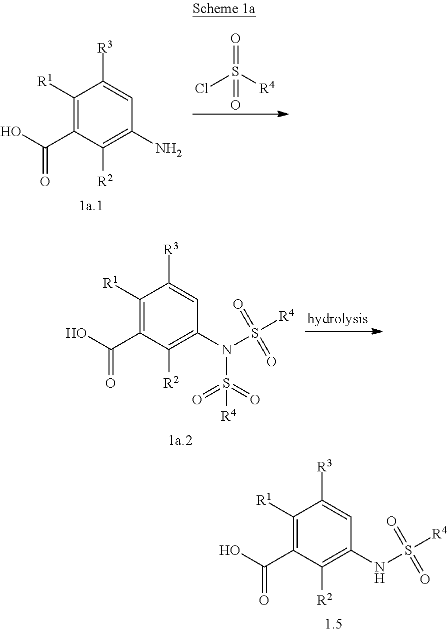

Scheme 1a shows an alternative procedure for the preparation of compounds 1.5. Aminobenzoic acid 1a.1 is treated with a sulfonyl chloride R⁴SO₂Cl in the presence of a base, such as NEt₃, in an organic solvent, such as dichloromethane (“DCM”). Hydrolysis of compound 1a.2 is accomplished under basic conditions, such as aqueous NaOH, in the appropriate solvent system, such as THF and/or MeOH, to provide the mono-sulfonamide 1.5.

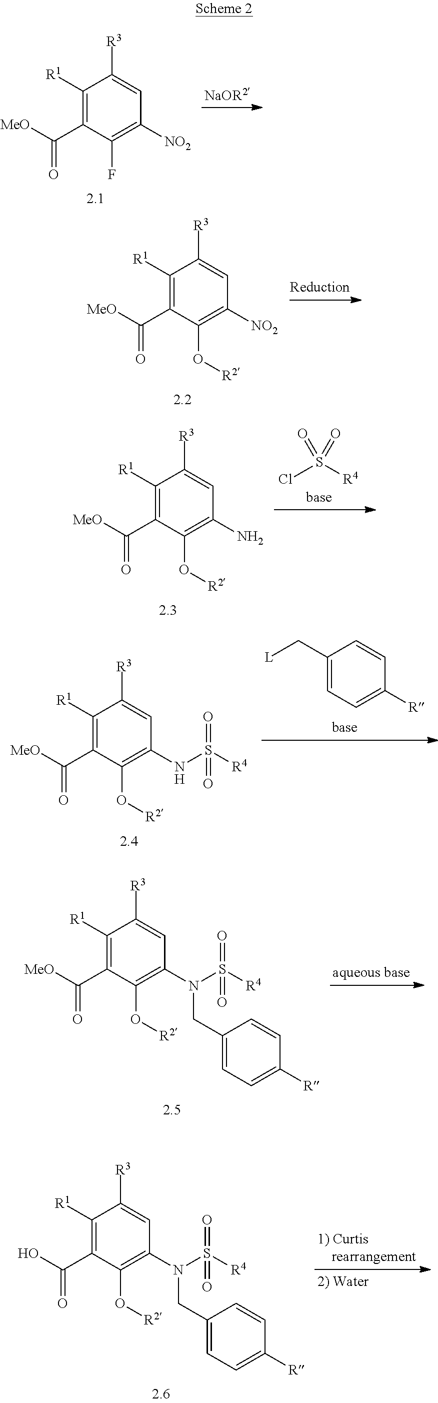

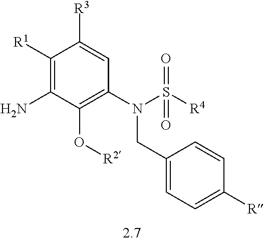

Scheme 2 describes the synthesis of aniline intermediates 2.7, wherein R¹, R²′, R³, R⁴ and R″ are as defined herein. A benzoic acid ester 2.1 is treated with an alkoxide NaOR^{2′} (wherein R^{2′} is C₁-C₃ alkyl) in an appropriate solvent, such as methanol, to form the ether intermediate 2.2. Reduction of the nitro group affords an aniline 2.3, which is reacted with a sulfonyl chloride R⁴SO₂Cl in the presence of base, such as pyridine, to give a sulfonamide intermediate 2.4. Benzylation with an optionally substituted benzyl halide, for example p-methoxybenzyl chloride, (wherein L is a leaving group such as chloro, bromo, iodo, triflate, tosylate; and R″ is hydrogen, C₁-C₃ alkyl or C₁-C₆ alkoxy; and in one example, R″ is hydrogen, in another example, R″ is OMe) in the presence of a base, such as sodium hydride, yields the protected sulfonamide ester 2.5, which is hydrolyzed with aqueous base, such as NaOH, to form the acid 2.6. In the last step, application of Curtius rearrangement conditions and subsequent hydrolysis gives the amino intermediate 2.7.

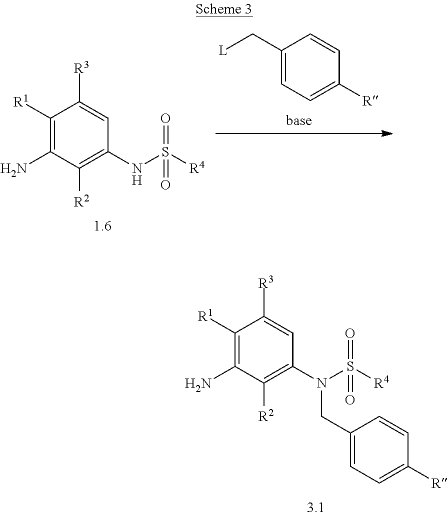

Scheme 3 shows a procedure for generating the aniline intermediate 3.1, wherein R″ and L are defined in Scheme 2 and R¹, R², R³ and R⁴ are as defined herein, through protection of the sulfonamide moiety of aniline 1.6. This transformation can be accomplished by treatment with an optionally substituted benzyl halide (e.g. p-methoxybenzyl chloride) and a base, such as sodium hydride.

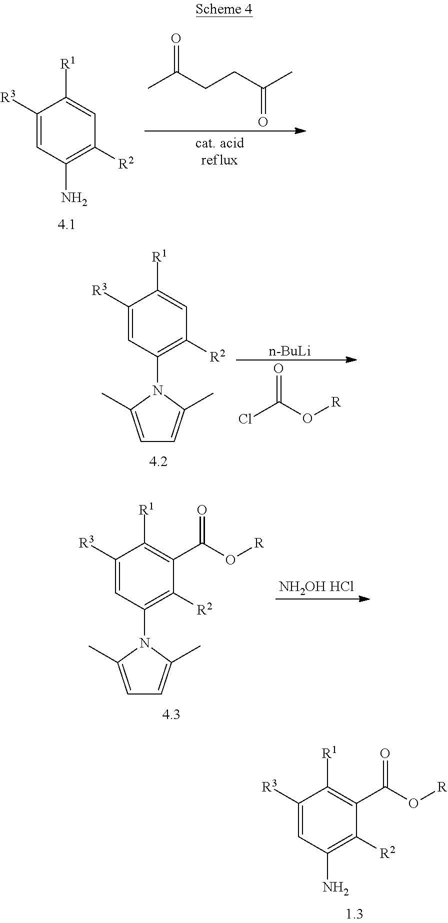

Scheme 4 describes the synthesis of an aniline ester of Formula 1.3, wherein R¹, R², and R³ are defined herein and R is alkyl, such as methyl or ethyl or benzyl. The amino group of an aniline 4.1 is protected by reacting with hexane-2,5-dione in the presence of a catalytic amount of an acid, such as p-toluenesulfonic acid, in a solvent, such as toluene, to form the 2,5-dimethylpyrrole derivative 4.2. Reaction with a carbamoyl chloride RO(C═O)Cl in the presence of n-butyllithium or a comparable agent in a suitable solvent, such as THF, leads to formation of the ester analog 4.3. The amino function of compound 4.3 is deprotected by reaction with hydroxylamine in a suitable solvent, such as ethanol, leading to formation of intermediate 1.3.

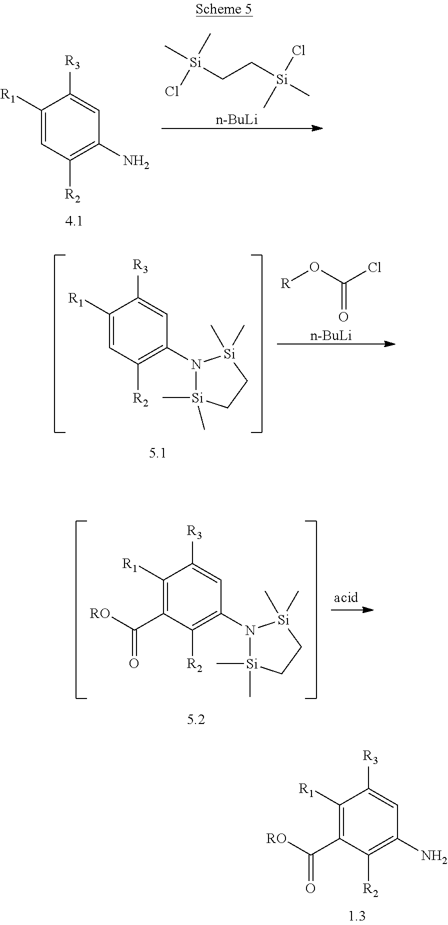

Scheme 5 describes the synthesis of an aniline ester of Formula 1.3, wherein R¹, R², and R³ are defined herein and R is alkyl, such as methyl or ethyl or benzyl. The amino group of an aniline 4.1 is protected by reacting with 1,2-bis(chlorodimethylsilyl)ethane in the presence of a strong base such as n-butyllithium in a suitable solvent, such as THF, at low temperatures, e.g. −78° C., to form the 1-aza-2,5-disilacyclopentane intermediate 5.1. Compound 5.1 is immediately reacted with a carbamoyl chloride RO(C═O)Cl in the presence of n-butyllithium or a comparable agent in a suitable solvent, such as THF, leading to formation of the ester analog 5.2. The amino function of compound 5.2 is deprotected by reaction with an acid such as HCl in a suitable solvent, leading to formation of intermediate 1.3.

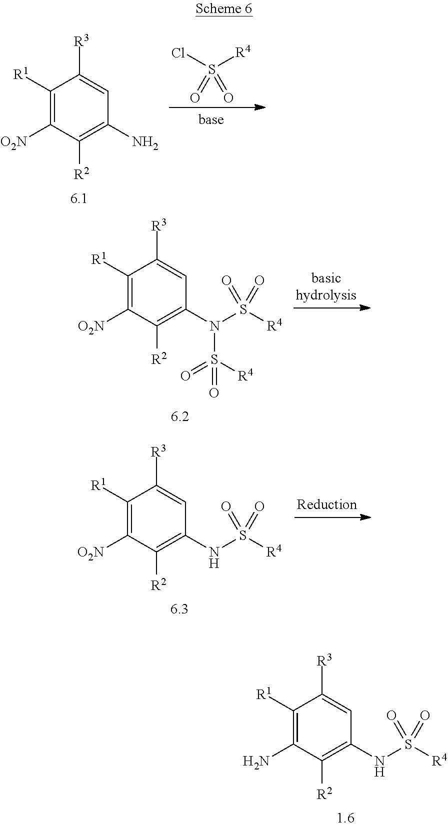

Scheme 6 describes another way of synthesizing an intermediate of Formula 1.6, wherein R¹, R², R³ and R⁴ are as defined herein. Bis-sulfonamide 6.2 is obtained by treatment of the aniline 6.1 with a sulfonyl chloride R⁴SO₂Cl in the presence of a base, such as NEt₃, in an organic solvent, such as dichloromethane. Hydrolysis of compound 6.2 is accomplished under basic conditions, such as aqueous NaOH, in the appropriate solvent system, such as THF and/or MeOH, to provide the mono-sulfonamide 6.3. This compound in a suitable solvent, such as ethanol, is treated with a reducing agent, such as iron and ammonium chloride to form an amine 1.6.

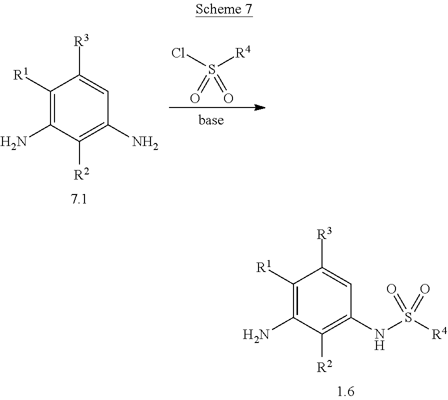

Scheme 7 shows another way of preparing an intermediate of Formula 1.6. This transformation is accomplished by mono-sulfonylation of a diamino derivative 7.1 with a sulfonyl chloride R⁴SO₂Cl in the presence of a base, such as pyridine, in an organic solvent, such as dichloromethane.

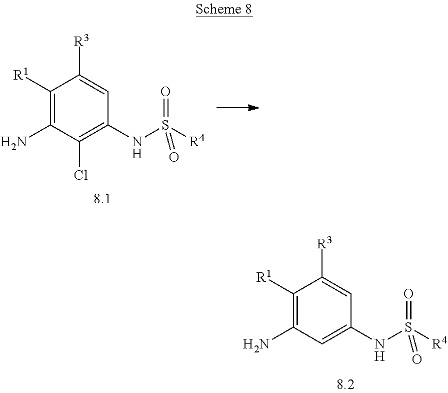

Scheme 8 describes the synthesis of an intermediate of Formula 8.2, a subset of Formula 1.6 compounds, wherein R³ and R⁴ are as defined herein and R² is hydrogen. This transformation is accomplished by reduction of the chloro atom of compound 8.1 using reducing conditions such as hydrogen in the presence of a palladium catalyst in a suitable solvent such as ethanol.

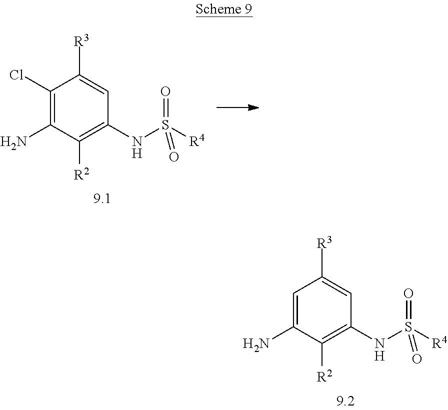

Scheme 9 describes the synthesis of an intermediate of Formula 9.2, a subset of Formula 1.6 compounds, wherein R², R³ and R⁴ are as defined herein and R¹ is hydrogen. This transformation is accomplished by reduction of the chloro atom of compound 9.1 using reducing conditions such as hydrogen in the presence of a palladium catalyst in a suitable solvent such as ethanol.

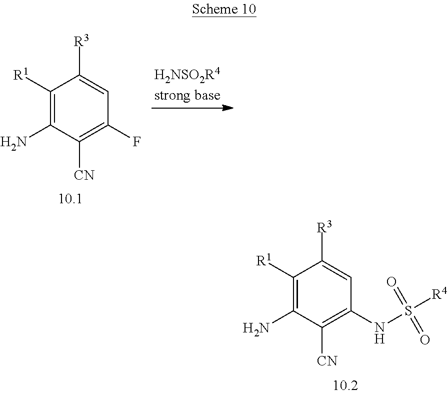

Scheme 10 shows a method for preparing nitrile-substituted aniline intermediates 10.2. Reaction of fluoronitrile 10.1 with the sodium salt of H₂NSO₂R⁴ (generated by a strong base such as sodium hydride) in a suitable solvent such as dimethylsulfoxide or N-methylpyrrolidone at elevated temperature, results in the formation of intermediate 10.2.

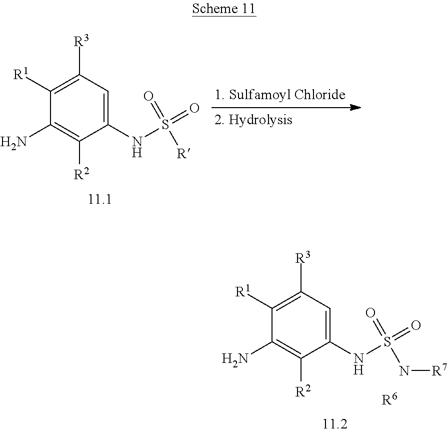

Scheme 11 shows a general method for preparing sulfamides of Formula 11.2, a subset of Formula 1.6 compounds, wherein R¹, R², R³, R⁶, and R⁷ are defined herein. A sulfonamide 11.1 (R′=alkyl), a subset of Formula 1.6 compounds, is treated with a sulfamoyl chloride in a solvent such as DMF and subsequently hydrolyzed to a sulfamide 11.2 by addition of a base and water, such as sodium hydroxide.

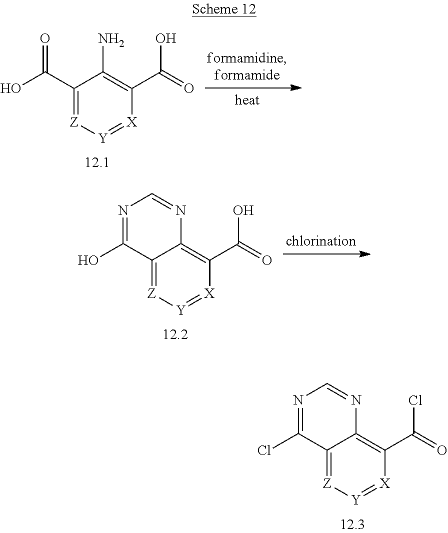

Scheme 12 describes the synthesis of an acid chloride 12.3. A bis-acid 12.1 is treated with formamidine and formamide at elevated temperature to afford the bicyclic intermediate 12.2. The aromatic OH and the acid moieties are chlorinated in the next step, for example, by using thionyl chloride and catalytic DMF, to give intermediate 12.3.

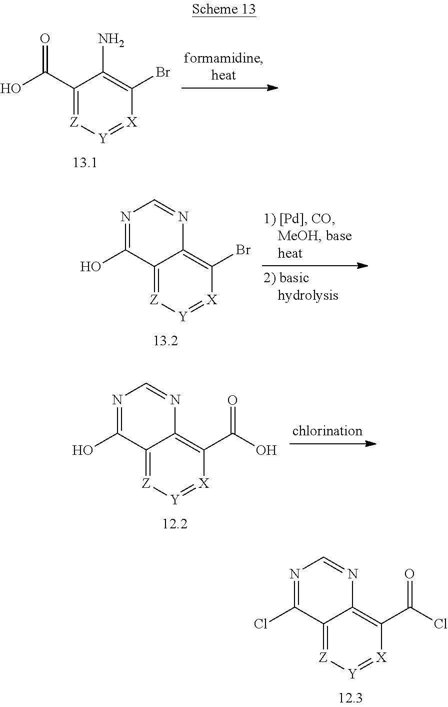

Scheme 13 describes another way of synthesizing an acid chloride 12.3. An acid 13.1 is treated with formamidine acetate at elevated temperature in a suitable solvent, such as ethanol, to afford the bicyclic intermediate 13.2. Introduction of a carboxylic acid moiety is accomplished through reaction with carbon monoxide, in the presence of a suitable palladium catalyst, such ad Pd(dppf)Cl₂, a base, such as triethylamine, and an alcoholic solvent, such as methanol, an subsequent hydrolysis using an inorganic base, such as sodium hydroxide, in water, and a suitable organic solvent, such as methanol or THF, to afford intermediate 12.2. The aromatic OH and the acid moieties are chlorinated in the next step, for example, by using thionyl chloride and catalytic DMF, to give compound 12.3.

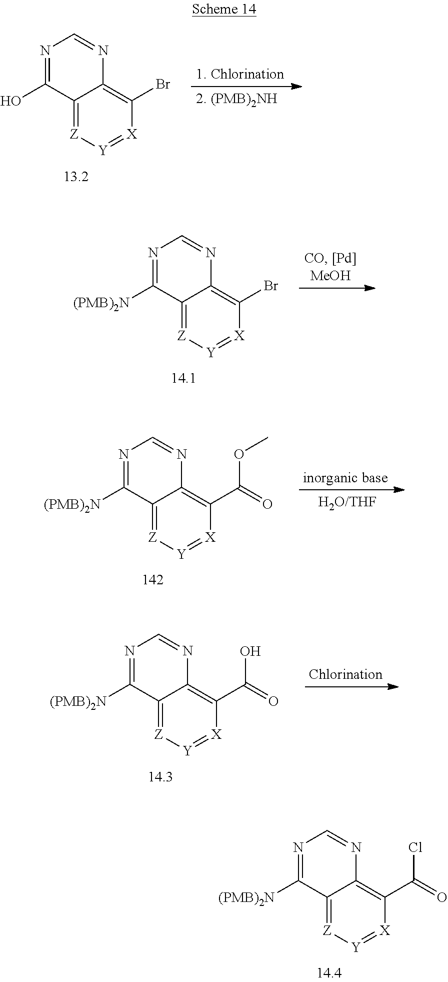

Scheme 14 shows a variation of Scheme 13, starting from intermediate 13.2. Chlorination, for example, by using thionyl chloride, and subsequent treatment with di-(p-methoxybenzyl)amine (“PMB” is p-methoxybenzyl) gives intermediate 14.1. Carbonylation reaction using carbon monoxide and a suitable Pd catalyst, for example Pd(PPh₃)₄, in the presence of a suitable protonating solvent, such as methanol, affords methyl ester 14.2. Ester 14.2 is hydrolyzed to acid 14.3 using a suitable base, such as NaOH, in aqueous THF, and subsequently converted to the corresponding acid chloride 14.4, for example, using thionyl chloride.

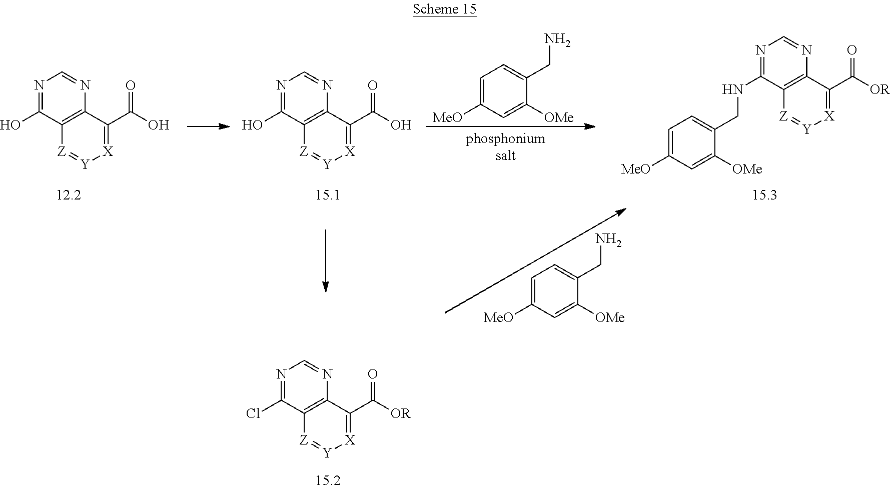

Scheme 15 shows a general procedure for obtaining the fused aminopyrimidine intermediate 15.3. Hydroxypyrimidine carboxylic acid 12.2 is esterified in an alcoholic solvent at reflux with an acid catalyst such as sulfuric acid. Hydroxypyrimidine ester 15.1 is converted to a dimethoxybenzyl-protected aminopyrimidine 15.3 via coupling with a suitable phosphonium salt such as BOP or PyBOP. Alternatively, compound 15.3 can be prepared via intermediate chloride 15.2, prepared from intermediate 15.1 with a chlorinating reagent such as thionyl chloride or phosphorus oxychloride.

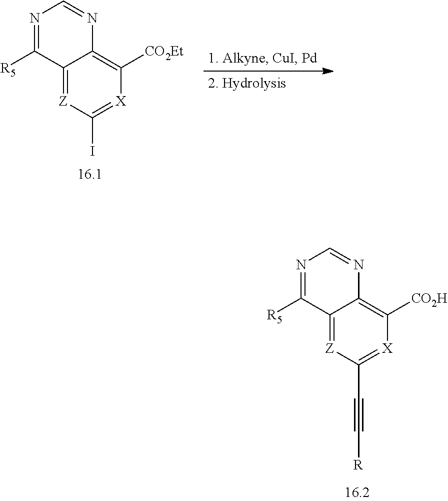

Scheme 16 shows a general procedure for obtaining intermediates of Formula 16.2. Starting with compounds of Formula 16.1, which are a subset of Formula 18.1 compounds, where Y is an iodine-substituted carbon, addition of an alkyne to the heteroaryl iodide can be accomplished using catalysts such as Pd and CuI in a suitable solvent, such as THF. Ester hydrolysis can be accomplished using a base such as lithium hydroxide in a solvent such as THF and water to afford compounds of Formula 16.2.

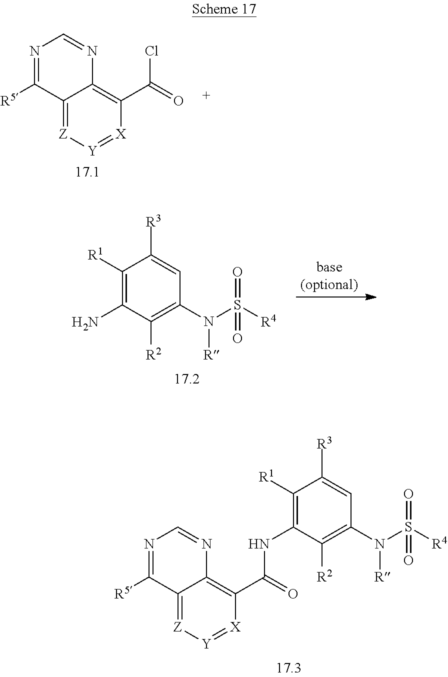

Scheme 17 shows a general procedure for obtaining compounds 17.3, wherein R¹, R², R³, R⁴, R⁵′ and R″ are as defined herein. An acid chloride 17.1 (wherein R^{5′} is R⁵, halogen or protected amine, for example mono- or di-(p-methoxybenzyl)amine) and an amine 17.2 (wherein R″ is hydrogen or a protecting group, for example di-(p-methoxybenzyl)amine) in a suitable solvent, such as chloroform or THF, and in the presence of an optional base, such as triethylamine or pyridine, are coupled to form compounds 17.3.

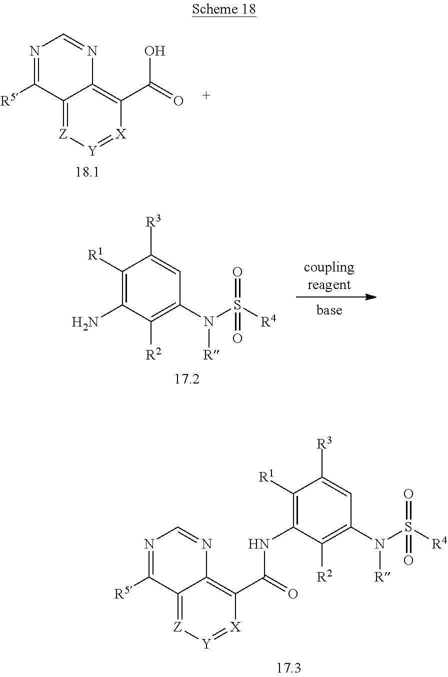

Scheme 18 describes another general procedure for obtaining compounds 17.3. Treatment of acid 18.1 with an activating agent such as (2-(7-Aza-1H-benzotriazole-1-yl)-1,1,3,3-tetramethyluronium hexafluorophosphate) (“HATU”) and a base such as N,N-diisopropylethylamine (“DIEA”) in an appropriate solvent such as DMF with amine 17.2 forms compounds of Formula 17.3.

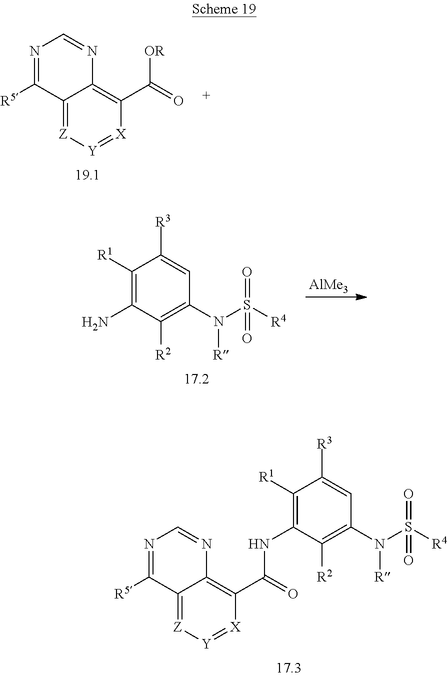

Scheme 19 describes another general procedure for obtaining compounds 17.3. Reaction of ester 18.1 (with R=small alkyl such as methyl or ethyl) with trimethylaluminum and amine 17.2 at elevated temperatures in an appropriate solvent such as toluene furnishes compounds of Formula 17.3.

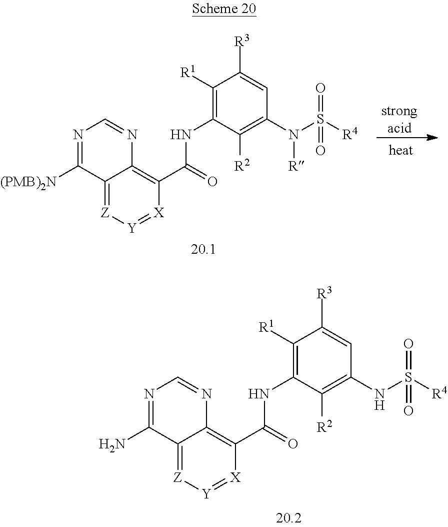

Scheme 20 describes the synthesis of compounds 20.2 from compounds 20.1, wherein R¹, R², R³, and R⁴ are as defined herein and R″ is a protecting group such as benzyl, monomethoxybenzyl or dimethoxybenzyl. Treatment of 20.1 with a strong acid, such as trifluoroacetic acid, under heat and optionally with microwave radiation forms compounds 20.2.

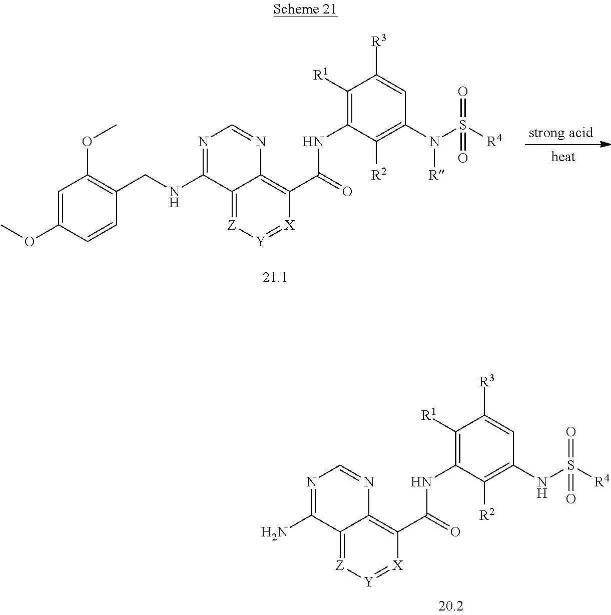

Scheme 21 describes another synthesis of compounds 20.2 from compounds 21.1, wherein R¹, R², R³, and R⁴ are as defined herein and R″ is a protecting group such as benzyl, monomethoxybenzyl or dimethoxybenzyl. Treatment of 21.1 with a strong acid, such as trifluoroacetic acid, under heat with microwave radiation forms compounds 20.2.

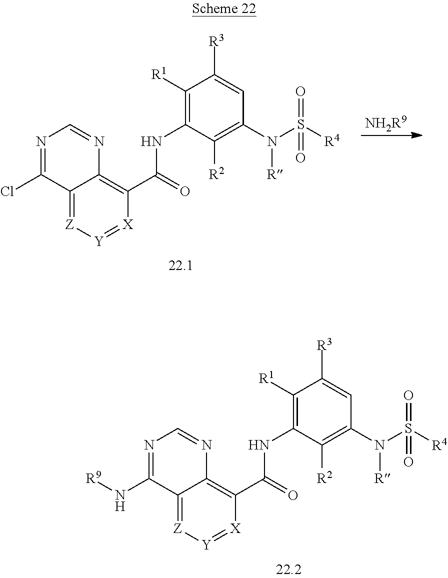

Scheme 22 shows a general procedure for obtaining compounds 21.2 from compounds 22.1, wherein R¹, R², R³, R⁴, R⁹ and R″ are as defined herein. This transformation is accomplished by treatment with an amine NH₂R⁹ in a suitable solvent, such as THF.

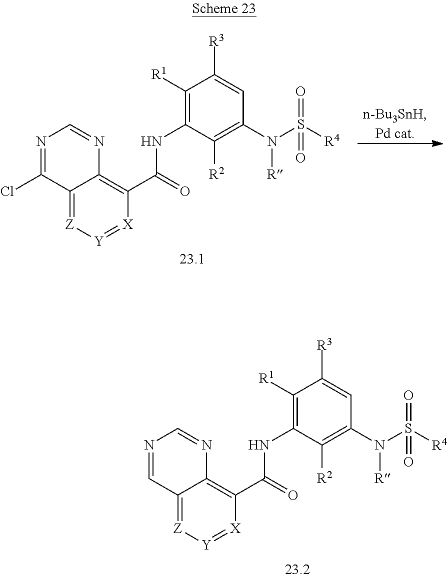

Scheme 23 shows a general procedure for obtaining compounds 23.2, wherein R¹, R², R³, R⁴ and R″ are as defined herein. Treatment of chloro compounds 23.1 with tributyltinhydride and a Pd catalyst in a suitable solvent, such as THF, affords compounds 23.2.

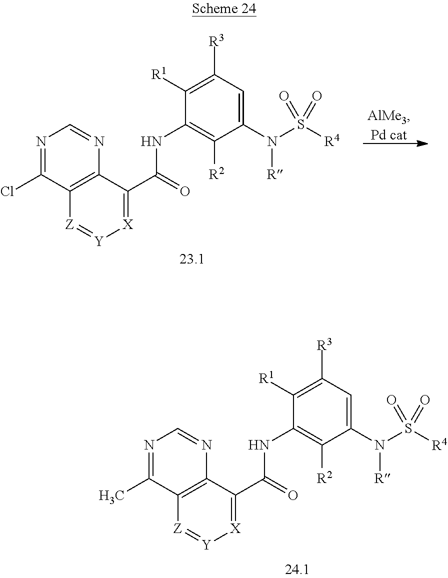

Scheme 24 shows a general procedure for obtaining compounds 24.1 wherein R¹, R², R³, R⁴, and R″ are as defined herein. Treatment of chloro compounds 23.1 with trimethylaluminum and a Pd catalyst, such as tetrakis(triphenylphosphine)palladium(0), in a suitable solvent, such as THF, affords compounds 24.1.

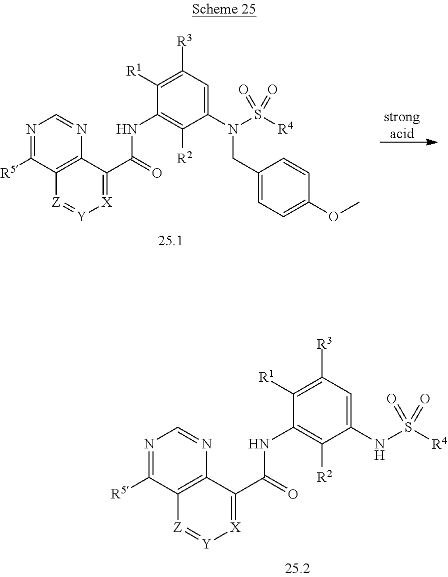

Scheme 25 shows a general procedure for the synthesis of compounds 25.2, wherein R¹, R², R³, R⁴ and R⁵′ are as defined herein. Using a strong acid, such as trifluoroacetic acid, in a suitable solvent, such as dichloromethane, 25.1 is deprotected to afford compounds 25.2.

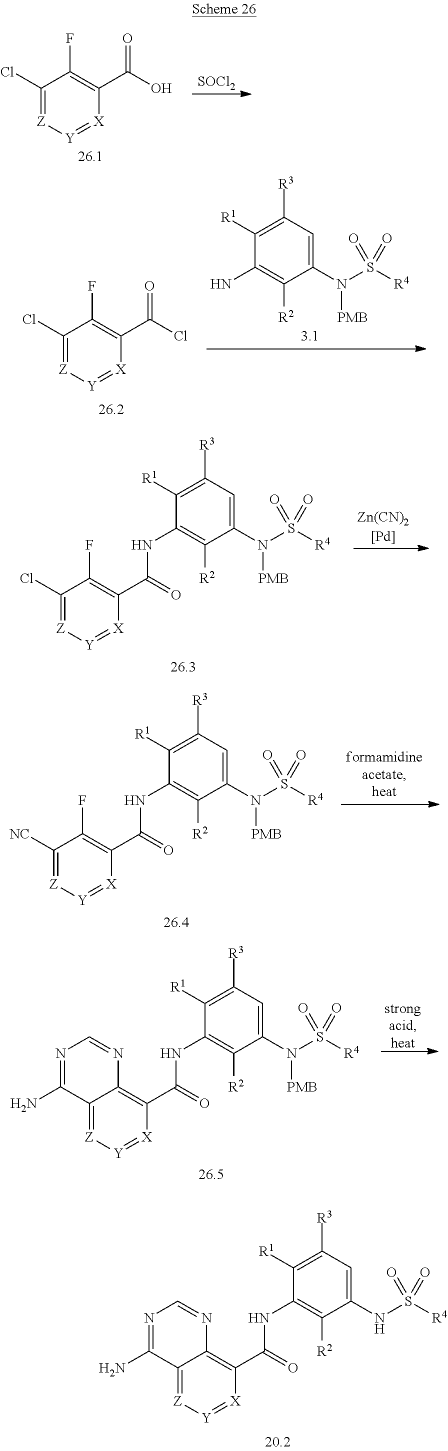

Scheme 26 describes an alternative method for the synthesis of compounds of Formula 20.2. A compound of Formula 26.1 is treated with a chlorinating agent such as thionyl chloride. Coupling of the resulting acid chloride 26.2 with an amino derivative of Formula 3.1 in a suitable solvent, such as chloroform, leads to formation of the amide 26.3. Conversion of the chlorine to a cyano group can be accomplished by treatment with zinc cyanide and a palladium catalyst, such as Pd(dppf)Cl₂, in a suitable solvent, such as DMF. Formation of a fused amino-substituted pyrimidine ring is accomplished by treatment of 26.4 with formamidine acetate at elevated temperatures in a suitable solvent, such as dimethylacetamide (“DMA”). Deprotection of the PMB group with a strong acid such as TFA leads to formation of the final product 20.2.

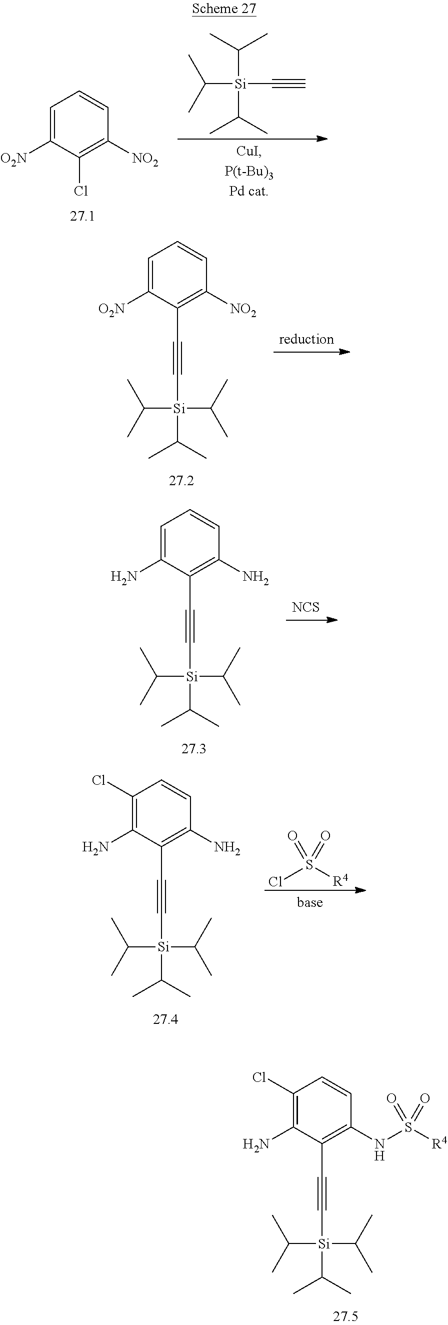

Scheme 27 describes the general synthesis of intermediates of Formula 27.5. 2-Chloro-1,3-dinitrobenzene (12.1), CuI, P(t-Bu)₃, and ethynyltriisopropylsilane and a Pd catalyst, such as PdCl₂(MeCN)₂, in a suitable solvent mixture, such as acetonitrile/triethylamine (5:1), are reacted to form the triisopropylsilane derivative 27.2. Reduction, for example using SnCl₂ in DCM/DMF (1:1), affords the corresponding diamine 27.3. Reaction with n-chlorosuccinimide (“NCS”) in a suitable solvent, such as THF, gived the chlorinated product 27.4, which is further transformed into sulfonamide 27.5 through reaction with a sulfonylchloride R⁴SO₂Cl.

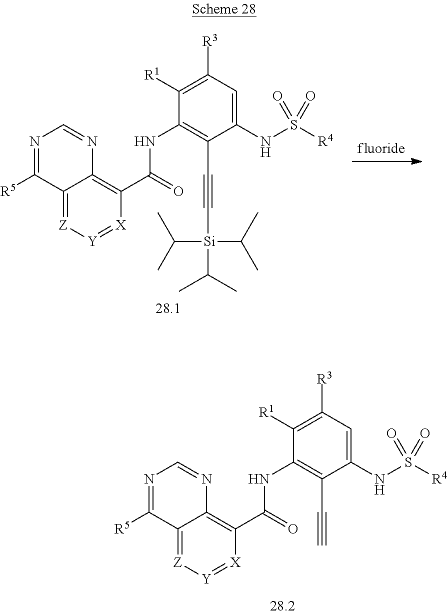

Scheme 28 describes the general synthesis of compounds of Formula 28.2, wherein R² is ethynyl. Trisopropylsilane-protected alkyne 28.1 is treated with a fluoride reagent, such as tetrabutylammonium fluoride (“TBAF”) in a suitable solvent, such as THF, to afford deprotected products of Formula 28.2.

Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-³³P]ATP into FSBA-modified wild-type MEK (see Example A).

Administration and Pharmaceutical Formulations

The compounds of the invention may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formulas I-VIII, or a stereoisomer or pharmaceutically acceptable salt thereof. In a further embodiment, the present invention provides a pharmaceutical composition comprising a compound of Formulas I-VIII, or a stereoisomer or pharmaceutically acceptable salt thereof, together with a pharmaceutically acceptable carrier or excipient.

Another embodiment of the present invention provides a pharmaceutical composition comprising a compound of Formulas I-VIII for use in the treatment of a hyperproliferative disease.

Another embodiment of the present invention provides a pharmaceutical composition comprising a compound of Formulas I-VIII for use in the treatment of cancer.

Another embodiment of the present invention provides a pharmaceutical composition comprising a compound of Formulas I-VIII for use in the treatment of kidney disease. A further embodiment of the present invention provides a pharmaceutical composition comprising a compound of Formulas I-VIII for use in the treatment of polycystic kidney disease.

Methods of Treatment with Compounds of the Invention

The invention includes methods of treating or preventing disease or condition by administering one or more compounds of this invention, or a stereoisomer or pharmaceutically acceptable salt thereof. In one embodiment, a human patient is treated with a compound of Formulas I-VIII, or a stereoisomer, tautomer or pharmaceutically acceptable salt thereof, and a pharmaceutically acceptable carrier, adjuvant, or vehicle in an amount to detectably inhibit B-Raf activity.

In another embodiment, a human patient is treated with a compound of Formulas I-VIII, or a stereoisomer, tautomer, prodrug or pharmaceutically acceptable salt thereof, and a pharmaceutically acceptable carrier, adjuvant, or vehicle in an amount to detectably inhibit B-Raf activity.

In another embodiment of the present invention, a method of treating a hyperproliferative disease in a mammal comprising administering a therapeutically effective amount of the compound of Formulas I-VIII, or a stereoisomer, tautomer, prodrug or pharmaceutically acceptable salt thereof, to the mammal is provided.

In another embodiment of the present invention, a method of treating a hyperproliferative disease in a mammal comprising administering a therapeutically effective amount of the compound of Formulas I-VIII, or a stereoisomer, tautomer or pharmaceutically acceptable salt thereof, to the mammal is provided.

In another embodiment of the present invention, a method of treating kidney disease in a mammal comprising administering a therapeutically effective amount of the compound of Formulas I-VIII, or a stereoisomer, tautomer, prodrug or pharmaceutically acceptable salt thereof, to the mammal is provided. In another embodiment of the present invention, a method of treating kidney disease in a mammal comprising administering a therapeutically effective amount of the compound of Formulas I-VIII, or a stereoisomer, tautomer or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formulas I-VIII, or a stereoisomer, tautomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment of the present invention provides the use of a compound of Formulas I-VIII, or a stereoisomer, tautomer or pharmaceutically acceptable salt thereof, in the manufacture of a medicament for the treatment of cancer.

In another embodiment, a method of treating or preventing cancer in a mammal in need of such treatment, wherein the method comprises administering to said mammal a therapeutically effective amount of a compound of Formulas I-VIII, or a stereoisomer, tautomer, prodrug or pharmaceutically acceptable salt thereof.

Another embodiment of the present invention provides the use of a compound of Formulas I-VIII, or a stereoisomer, tautomer, prodrug or pharmaceutically acceptable salt thereof, in the manufacture of a medicament for the treatment of cancer.

Another embodiment of the present invention provides the use of a compound of Formulas I-VIII, or a stereoisomer, tautomer, prodrug or pharmaceutically acceptable salt thereof, in the manufacture of a medicament for the treatment of kidney disease. Another embodiment of the present invention provides the use of a compound of Formulas I-VIII, or a stereoisomer, tautomer or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formulas I-VIII, or a stereoisomer, tautomer, prodrug or pharmaceutically acceptable salt thereof, alone or in combination with one or more additional compounds having anti-cancer properties.

In another embodiment, a method of preventing or treating cancer, comprising administering to a mammal in need of such treatment an effective amount of a compound of Formulas I-VIII, or a stereoisomer, tautomer or pharmaceutically acceptable salt thereof, alone or in combination with one or more additional compounds having anti-cancer properties.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

In another embodiment, a method of treating or preventing a disease or disorder modulated by B-Raf, comprising administering to a mammal in need of such treatment an effective amount of a compound of Formulas I-VIII, or a stereoisomer, tautomer or pharmaceutically acceptable salt thereof. Examples of such diseases and disorders include, but are not limited to, cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In another embodiment, a method of treating or preventing a disease or disorder modulated by B-Raf, comprising administering to a mammal in need of such treatment an effective amount of a compound of Formulas I-VIII, or a stereoisomer, tautomer, prodrug or pharmaceutically acceptable salt thereof.

In another embodiment of the present invention, a method of preventing or treating kidney disease, comprising administering to a mammal in need of such treatment an effective amount of a compound of Formulas I-VIII, or a stereoisomer, tautomer, prodrug or pharmaceutically acceptable salt thereof, alone or in combination with one or more additional compounds. In another embodiment of the present invention, a method of preventing or treating polycystic kidney disease, comprising administering to a mammal in need of such treatment an effective amount of a compound of Formulas I-VIII, or a stereoisomer, tautomer, prodrug or pharmaceutically acceptable salt thereof, alone or in combination with one or more additional compounds.

Another embodiment of the present invention provides the use of a compound of Formulas I-VIII, or a stereoisomer, tautomer or pharmaceutically acceptable salt thereof, in the manufacture of a medicament for the treatment of cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. In a further embodiment, the use of a compound of Formulas I-VIII in the manufacture of a medicament, for use as a b-Raf inhibitor in the treatment of a patient undergoing cancer therapy.

Another embodiment of the present invention provides the use of a compound of Formulas I-VIII, or a stereoisomer, tautomer, prodrug or pharmaceutically acceptable salt thereof, in the manufacture of a medicament for the treatment of cancer.

Another embodiment of the present invention provides the use of a compound of Formulas I-VIII, or a stereoisomer, tautomer, prodrug or pharmaceutically acceptable salt thereof, in the manufacture of a medicament for the treatment of polycystic kidney disease. In a further embodiment, the kidney disease is polycystic kidney disease.

Another embodiment of the present invention provides the compounds of Formulas I-VIII for use in therapy.

Another embodiment of the present invention provides the compounds of Formulas I-VIII for use in the treatment of a hyperproliferative disease. In a further embodiment, the hyperproliferative disease is cancer (as further defined and may be individually selected from those above).

Another embodiment of the present invention provides the compounds of Formulas I-VIII for use in the treatment of kidney disease. In a further embodiment, the kidney disease is polycystic kidney disease.

Combination Therapy

The compounds of this invention, stereoisomers, tautomers and pharmaceutically acceptable salts thereof may be employed alone or in combination with other therapeutic agents for treatment. The compounds of the present invention can be used in combination with one or more additional drugs, for example an anti-hyperproliferative, anti-cancer, or chemotherapeutic agent. The second compound of the pharmaceutical combination formulation or dosing regimen preferably has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) PI3k/AKT/mTOR pathway inhibitors, including GDC-0941 (2-(1H-Indazol-4-yl)-6-(4-methanesulfonyl-piperazin-1-ylmethyl)-4-morpholin-4-yl-thieno[3,2-d]pyrimidine), XL-147, GSK690693 and temsirolimus; (xi) Ras/Raf/MEK/ERK pathway inhibitors; and (xii) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

In order to illustrate the invention, the following Examples are included. However, it is to be understood that these Examples do not limit the invention and are only meant to suggest a method of practicing the invention. Persons skilled in the art will recognize that the chemical reactions described may be readily adapted to prepare a number of other compounds of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography purification was done on a Biotage system (Manufacturer: Biotage AB) having a silica gel column or on a silica SepPak cartridge (Waters) or on a Teledyne Isco Combiflash purification system using prepacked silica gel cartridges. ¹H NMR spectra were recorded on a Bruker AVIII 400 MHz or Bruker AVIII 500 MHz or on a Varian 400 MHz NMR spectrometer.

¹H-NMR spectra were obtained as CDCl₃, CD₂Cl₂, CD₃OD, D₂O, DMSO-d₆, acetone-d₆ or CD₃CN solutions (reported in ppm), using tetramethylsilane (0.00 ppm) or residual solvent (CDCl₃: 7.25 ppm; CD₃OD: 3.31 ppm; D₂O: 4.79 ppm; d₆-DMSO: 2.50 ppm; acetone-d₆: 2.05 ppm; CD₃CN: 1.94 ppm) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

### Biological Example A

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-³³P]ATP into FSBA-modified wild-type MEK. The 30-μL assay mixtures contained 25 mM Na Pipes, pH 7.2, 100 mM KCl, 10 mM MgCl₂, 5 mM β-glycerophosphate, 1000/1 Na Vanadate, 4 μM ATP, 500 nCi [γ-³³P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5×(20 μL of 30 nM and 1.5 μM, respectively) for 15 minutes, and the assay was initiated by the addition of 10 μL of 10 μM ATP. Following the 60-minute incubation, the assay mixtures were quenched by the addition of 100 μL of 25% TCA, the plate was mixed on a rotary shaker for 1 minute, and the product was captured on a Perkin-Elmer GF/B filter plate using a Tomtec Mach III Harvester. After sealing the bottom of the plate, 35 μL of Bio-Safe II (Research Products International) scintillation cocktail were added to each well and the plate was top-sealed and counted in a Topcount NXT (Packard).

The compounds of Examples 1-71 and 73-79 were tested in the above assay, and compounds of Examples 1-40, 43, 45, 47, 49-70, 73 and 77-79 were found to have an IC₅₀ of less than 1 μM.

The compounds of Examples 1-3, 5-40, 55, 57-70 and 77-79 were tested in the above assay and found to have an IC₅₀ of less than 100 nM.

### PS Biological Example A1

### Cellular ERK 1/2 Phosphorylation Assay

Inhibition of basal ERK1/2 phosphorylation was determined by the following in vitro cellular proliferation assay, which comprises incubating cells with a compound of Formula I for 1 hour and quantifying the fluorescent pERK signal on fixed cells and normalizing to total ERK signal.

Materials and Methods:

Malme-3M cells were obtained from ATCC and grown in RPMI-1640 supplemented with 10% fetal bovine serum. Cells were plated in 96-well plates at 24,000 cells/well and allowed to attach for 16-20 hours at 37° C., 5% CO₂. The media was removed, and DMSO-diluted compounds were added in RPMI-1640 at a final concentration of 1% DMSO. The cells were incubated with the compounds for 1 hour at 37° C., 5% CO₂. The cells were washed with PBS and fixed in 3.7% formaldehyde in PBS for 15 minutes. This was followed by washing in PBS/0.05% Tween20 and permeabilizing in −20° C. 100% MeOH for 15 minutes. Cells were washed in PBS/0.05% Tween20 then blocked in Odyssey blocking buffer (LI-COR Biosciences) for 1 hour. Antibodies to phosphorylated ERK (1:400, Cell Signaling #9106, monoclonal) and total ERK (1:400, Santa Cruz Biotechnology #sc-94, polyclonal) were added to the cells and incubated 16-20 hours at 4° C. After washing with PBS/0.05% Tween20, the cells were incubated with fluorescently-labeled secondary antibodies (1:1000 goat anti-rabbit IgG-IRDye800, Rockland and 1:500 goat anti-mouse IgG-Alexa Fluor 680, Molecular Probes) for an additional hour. Cells were then washed and analyzed for fluorescence at both wavelengths using the Odyssey Infrared Imaging System (LI-COR Biosciences). Phosphorylated ERK signal was normalized to total ERK signal.

### Biological Example A2

### Tumor Growth Inhibition (LOX)

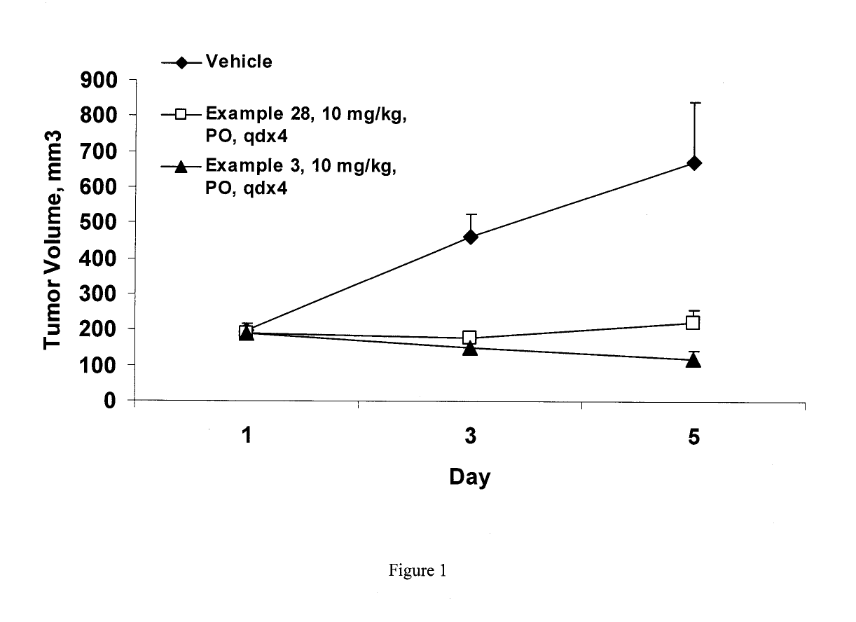

Female nude mice were implanted subcutaneously on the right flank with approximately 3.5×10⁶ LOX cells in 100 μL PBS. Five to seven days later, tumors were measured and mice were randomized into groups of six with average tumor volume in each group of approximately 200 mm³. Examples 3 and 28 were dissolved in 80% PEG400/20% ethanol before dosing, and administered PO at a volume of 5 mL/kg. Dosing was vehicle alone on days 1, 2, 3 and 4; and Examples 3 and 28 at 10 mg/kg on days 1, 2, 3 and 4. Animal weights and tumor volumes were measured using electronic calipers on day 5. Tumor volume was calculated using the formula: volume=(width2×length)/2. The results are shown in FIG. 1 and the Table below.

| | Tumor Volume | Tumor Volume | Tumor Volume |
|---|---|---|---|
| | (mm³) | (mm³) | (mm³) |
| Compound | Day 1 | Day 3 | Day 5 |
| Vehicle | 196.97 | 460.42 | 672.85 |
| Example 3 | 190.33 | 151.33 | 118.41 |
| Example 28 | 190.71 | 176.38 | 219.31 |

### Biological Example A3

### Tumor Growth Inhibition (LOX)

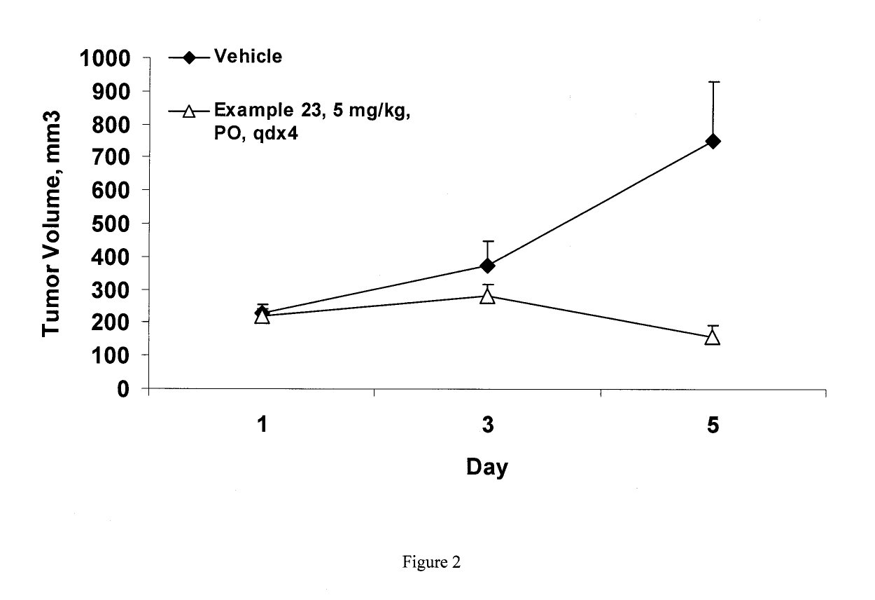

Female nude mice were implanted subcutaneously on the right flank with approximately 3.5×10⁶ LOX cells in 100 μL PBS. Five to seven days later, tumors were measured and mice were randomized into groups of six with average tumor volume in each group of approximately 200 mm³. Example 23 was dissolved in 80% PEG400/20% ethanol before dosing, and administered PO at a volume of 5 mL/kg. Dosing was vehicle alone on days 1, 2, 3 and 4; and Example 23 at 5 mg/kg on days 1, 2, 3 and 4. Animal weights and tumor volumes were measured using electronic calipers on day 5. Tumor volume was calculated using the formula: volume=(width²×length)/2. The results are shown in FIG. 2 and the table below.

| | Tumor Volume | Tumor Volume | Tumor Volume |
|---|---|---|---|
| | (mm³) | (mm³) | (mm³) |
| Compound | Day 1 | Day 3 | Day 5 |
| Vehicle | 226.24 | 372.51 | 749.2 |
| Example 23 | 221.32 | 280.81 | 157.52 |

### Example B

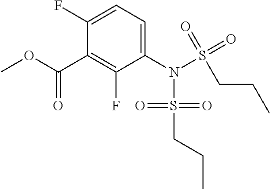 [SMILES: CCCS(=O)(=O)N(c1ccc(F)c(C(=O)OC)c1F)S(=O)(=O)CCC]

### Methyl 2,6-difluoro-3-(N-(propylsulfonyl)propylsulfonamido)benzoate

Step A:

A 1 L flask was charged with 2,6-difluoro-3-nitrobenzoic acid (17.0 g, 83.7 mmol) and MeOH (170 mL, 0.5M). The flask was placed in a cold water bath, and an addition funnel charged with a 2M solution of trimethylsilyl (“TMS”) diazomethane in hexanes (209 mL, 419 mmol) was attached to the flask. The TMS diazomethane solution was added slowly to the reaction flask over the course of 2 hours. A large excess of reagent was required in order for the reaction to reach completion as determined by the ceased evolution of N₂ upon further addition of reagent. The volatiles were removed in vacuo to afford crude methyl 2,6-difluoro-3-nitrobenzoate as a solid (18.2 g). The material was taken directly to Step B.

Step B:

10% (wt.) Pd on activated carbon (4.46 g, 4.19 mmol) was added to a 1 L flask charged with methyl 2,6-difluoro-3-nitrobenzoate (18.2 g, 83.8 mmol) under a nitrogen atmosphere. To the flask was added EtOH (350 mL, 0.25 M), and H₂ gas was passed through the mixture for 15 minutes. The reaction mixture was stirred under two H₂ balloons overnight. The balloons were recharged with H₂ gas and the mixture was stirred an additional 4 hours. Upon consumption of the starting material and intermediate hydroxylamine as determined by TLC, N₂ gas was flushed through the reaction mixture. The mixture was then filtered through glass microfibre filter (“GF/F”) paper twice. The volatiles were removed to afford crude methyl 3-amino-2,6-difluorobenzoate as an oil (15.66 g). The material was taken directly onto the next step.

Step C:

Propane-1-sulfonyl chloride (23.46 mL, 209.3 mmol) was slowly added to a solution of methyl 3-amino-2,6-difluorobenzoate (15.66 g, 83.7 mmol) and triethylamine (35.00 mL, 251.1 mmol) in CH₂Cl₂ (175 mL, 0.5M) maintained in a cool water bath. The reaction mixture was stirred for 1 hour at room temperature. Water (300 mL) was added and the organic layer was separated, washed with water (2×300 mL) and brine (200 mL), then dried (Na₂SO₄), filtered and concentrated to an oil. The crude product was purified by column chromatography, eluting with 15% ethyl acetate (“EtOAc”)/hexane. The isolated fractions were triturated with hexanes to afford methyl 2,6-difluoro-3-(N-(propylsulfonyl)propylsulfonamido)benzoate as a solid (24.4 g, 73% yield for 3 steps). ¹H NMR (400 MHz, CDCl₃) δ 7.52-7.45 (m, 1H), 7.08-7.02 (m, 1H), 3.97 (s, 3H), 3.68-3.59 (m, 2H), 3.53-3.45 (m, 2H), 2.02-1.89 (m, 4H), 1.10 (t, J=7.4 Hz, 6H). m/z (APCI-neg) M-(SO₂Pr)=292.2.

### Example C

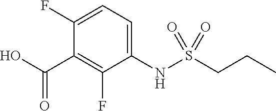 [SMILES: CCCS(=O)(=O)Nc1ccc(F)c(C(=O)O)c1F]

### 2,6-Difluoro-3-(propylsulfonamido)benzoic acid

A 1N aqueous NaOH solution (150 mL, 150 mmol) was added to a solution of methyl 2,6-difluoro-3-(N-(propylsulfonyl)propylsulfonamido)benzoate (20.0 g, 50.1 mmol) in 4:1 THF/MeOH (250 mL, 0.2M). The reaction mixture was stirred at room temperature overnight. The majority of the organic solvents were removed in vacuo (water bath temperature 35° C.). 1N HCl (150 mL) was slowly added to the mixture, and the resulting solid was filtered and rinsed with water (4×50 mL). The material was washed with Et₂O (4×15 mL) to give 2,6-difluoro-3-(propylsulfonamido)benzoic acid as a solid (10.7 g, 77% yield). ¹H NMR (400 MHz, d₆-DMSO) δ 9.74 (s, 1H), 7.57-7.50 (m, 1H), 7.23-7.17 (m, 1H), 3.11-3.06 (m, 2H), 1.79-1.69 (m, 2H), 0.98 (t, J=7.4 Hz, 3H). m/z (APCI-neg) M−1=278.0.

### Example D

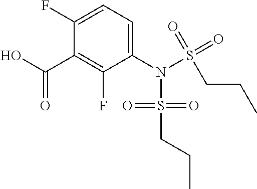 [SMILES: CCCS(=O)(=O)N(c1ccc(F)c(C(=O)O)c1F)S(=O)(=O)CCC]

### 2,6-Difluoro-3-(N-(propylsulfonyl)propylsulfonamido)benzoic acid

Propane-1-sulfonyl chloride (1.225 mL, 10.92 mmol) was added to a mixture of 3-amino-2,6-difluorobenzoic acid (0.573 g, 3.310 mmol), triethylamine (2.030 mL, 14.56 mmol) and CH₂Cl₂ (17 mL, 0.2M) cooled to 0° C. The reaction mixture was allowed to warm to room temperature and stirred for 1 hour. The mixture was then partitioned between saturated NaHCO₃ (100 mL) and ethyl acetate (75 mL). The aqueous layer was washed with ethyl acetate (50 mL) and then acidified with concentrated HCl to a pH of about 1. The acidified aqueous layer was extracted with ethyl acetate (2×50 mL), and the combined ethyl acetate extracts were dried (over Na₂SO₄), filtered and concentrated. The resulting residue was triturated with hexanes to afford 2,6-difluoro-3-(N-(propylsulfonyl)propyl-sulfonamido)benzoic acid as a solid (0.948 g, 74% yield). ¹H NMR (400 MHz, DMSO-d₆) δ 7.90-7.84 (m, 1H), 7.39-7.34 (m, 1H), 3.73-3.58 (m, 4H), 1.88-1.74 (m, 4H), 1.01 (t, J=7.5 Hz, 6H). m/z (APCI-neg) M−(SO₂Pr)=278.1.

### Example E

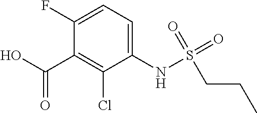 [SMILES: CCCS(=O)(=O)Nc1ccc(F)c(C(=O)O)c1Cl]

### 2-Chloro-6-fluoro-3-(propylsulfonamido)benzoic acid

Step A:

Into a 20-L 4-neck round flask was placed a solution of 2-chloro-4-fluorobenzenamine (1300 g, 8.82 mol, 1.00 equiv, 99%) in toluene (10 L), 4-methylbenzenesulfonic acid (3.1 g, 17.84 mmol, 99%), and hexane-2,5-dione (1222.5 g, 10.62 mol, 1.20 equiv, 99%). The resulting solution was heated to reflux for 1 h in an oil bath and cooled. The pH value of the solution was adjusted to 8 with sodium carbonate (1 mol/L). The resulting mixture was washed with 1×5000 mL of water and concentrated under vacuum. The crude product was purified by distillation and the fraction was collected at 140° C. to afford 1-(2-chloro-4-fluorophenyl)-2,5-dimethyl-1H-pyrrole (1700 g, yield: 85%).

Step B:

Into a 5000-mL 4-necked round-bottom flask purged and maintained with an inert atmosphere of nitrogen was placed a solution of 1-(2-chloro-4-fluorophenyl)-2,5-dimethyl-1H-pyrrole (390 g, 1.65 mol, 1.00 equiv, 95%) in tetrahydrofuran (2000 mL). The reaction vessel was cooled to −78° C. To the above reaction vessel was added n-BuLi (800 mL, 1.10 equiv, 2.5%) dropwise with stirring over 80 minutes and methyl carbonochloridate (215.5 g, 2.27 mol, 1.20 equiv, 99%) dropwise with stirring over 90 minutes. The reaction solution was further stirred for 60 minutes at −78° C. and quenched by the addition of 1000 mL of NH₄Cl/water. The resulting solution was extracted with 1500 mL of ethyl acetate. The organic layers were combined, washed with 1×1500 mL of water and 1×1500 mL of sodium chloride(aq), dried over anhydrous magnesium sulfate, and concentrated under vacuum to afford methyl 2-chloro-3-(2,5-dimethyl-1H-pyrrol-1-yl)-6-fluorobenzoate (crude, 566.7 g).

Step C:

Into five 5000-mL 4-neck round-bottom flasks was placed a solution of methyl 2-chloro-3-(2,5-dimethyl-1H-pyrrol-1-yl)-6-fluorobenzoate (1500 g, 5.05 mol, 1.00 equiv, 95%) in ethanol/H₂O (7500/2500 mL), NH₂OH—HCl (5520 g, 79.20 mol, 15.00 equiv, 99%), and triethylamine (2140 g, 20.98 mol, 4.00 equiv, 99%). The resulting solution was refluxed for 18 h in an oil bath, cooled to room temperature, concentrated, and extracted with 3×3000 mL of ethyl acetate. The organic layers were combined, dried over anhydrous sodium sulfate, and concentrated under vacuum. The residue was purified using a silica gel column eluting with PE:EA (20:1-10:1) to afford methyl 3-amino-2-chloro-6-fluorobenzoate (980 g, yield: 95%).

Step D:

Into four 5000-mL 4-neck round-bottom flasks was placed a solution of methyl 3-amino-2-chloro-6-fluorobenzoate (980 g, 4.76 mol, 1.00 equiv, 99%) in dichloromethane (8000 mL). Triethylamine (1454 g, 14.25 mol, 3.00 equiv, 99%) was added dropwise with stirring at 0° C. over 80 minutes followed by the addition of propane-1-sulfonyl chloride (1725 g, 11.94 mol, 2.50 equiv, 99%). The resulting solution was stirred at room temperature for 2 h, diluted with 1000 mL of water. The organic layer was washed with 1×1000 mL of hydrogen chloride and 1×1000 mL of water, dried over sodium sulfate, and concentrated to afford methyl 2-chloro-6-fluoro-3-(propylsulfonamido)benzoate as a brown solid (1500 g, 97%).

Step E:

Into a 10000-mL 4-necked round-bottom flask was placed a solution of methyl 2-chloro-6-fluoro-3-(propylsulfonamido)benzoate (1500 g, 4.61 mol, 1.00 equiv, 95%) in tetrahydrofuran/H₂O (3000/3000 mL) and potassium hydroxide (1000 g, 17.68 mol, 4.50 equiv, 99%). The resulting solution was refluxed for 2 hours, cooled to room temperature and extracted with 3×2000 mL of ethyl acetate. The aqueous layers were combined and the pH was adjusted to 2 with hydrogen chloride (2 mol/L). The resulting solution was extracted with 2×3000 mL of dichloromethane. The organic layers were combined, dried over anhydrous sodium sulfate and concentrated to afford 2-chloro-6-fluoro-3-(propylsulfonamido)benzoic acid (517.5 g, yield: 37%). (ES, m/z): [M+H]⁺296. ¹H NMR (400 MHz, CDCl₃): δ 1.058-1.096 (m, J=15.2 Hz, 3H), 1.856-1.933 (m, 2H), 3.073-3.112 (m, 2H); 6.811 (1H, s), 7.156-7.199 (d, J=17.2 Hz, 1H), 7.827-7.863 (d, J=14.4 Hz, 1H).

### Example F

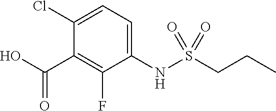 [SMILES: CCCS(=O)(=O)Nc1ccc(Cl)c(C(=O)O)c1F]

### 6-Chloro-2-fluoro-3-(propylsulfonamido)benzoic acid

Step A:

A flame dried flask equipped with a stir bar and rubber septum was charged with 4-chloro-2-fluoroaniline (5.00 g, 34.35 mmol) and anhydrous THF (170 mL). This solution was chilled to −78° C., and n-BuLi (14.7 mL, 1.07 eq. of 2.5M solution in hexanes) was then added over a 15 minute period. This mixture was stirred at −78° C. for 20 minutes, and then a THF solution (25 mL) of 1,2-bis(chlorodimethylsilyl)ethane (7.76 g, 1.05 eq.) was added slowly (over a 10 minute period) to the reaction mixture. This was stirred for 1 hour, and then 2.5M n-BuLi in hexanes (15.11 mL, 1.1 eq.) was added slowly. After allowing the mixture to warm to room temperature for one hour, the mixture was chilled back to −78° C. A third allotment of n-BuLi (15.66 mL, 1.14 eq.) was added slowly, and the mixture was stirred at −78° C. for 75 minutes. Benzyl chloroformate (7.40 g, 1.2 eq.) was then added slowly, and the mixture was stirred at −78° C. for one hour. The cooling bath was then removed. The mixture was allowed to warm for 30 minutes and then quenched with water (70 mL) and concentrated HCl (25 mL). The mixture was allowed to continue to warm to room temperature. The mixture was then extracted with EtOAc. The extracts were washed twice with a saturated Na₂HCO₃ solution, once with water, dried over sodium sulfate and concentrated. The resulting residue was flashed on a 65 Biotage (30% ethyl acetate/hexane) to produce benzyl 3-amino-6-chloro-2-fluorobenzoate (4.3 g, 45%) as an oil. ¹H NMR (DMSO-d₆, 400 MHz) δ 7.37-7.48 (m, 5H), 7.07 (dd, J=8, 2 Hz, 1H), 6.87 (t, J=8 Hz, 1H), 5.61 (br s, 2H), 5.40 (s, 2H).

Step B:

Benzyl 3-amino-6-chloro-2-fluorobenzoate (4.3 g, 15.37 mmol) was dissolved in dry dichloromethane (270 mL). Triethylamine (5.36 mL, 2.5 eq.) was added, and the mixture was chilled to 0° C. Propane-1-sulfonyl chloride (3.63 mL, 32.3 mmol, 2.1 eq.) was then added via syringe, and a precipitate resulted. Once the addition was complete, the mixture was allowed to warm to room temperature, and the starting material was consumed as determined by TLC (3:1 hexane:ethyl acetate). The mixture was then diluted with dichloromethane (200 mL), washed with 2M aqueous HCl (2×100 mL), saturated NaHCO₃ solution, dried over sodium sulfate and concentrated. The resulting residue was purified on a 65 Biotage chromatography system (40% ethyl acetate/hexane) to produce benzyl 6-chloro-2-fluoro-3-(N-(propylsulfonyl)propylsulfonamido)benzoate (5.5 g, 72%) as an oil that slowly solidified upon standing. NMR (CDCl₃, 400 MHz) δ 7.28-7.45 (m, 7H), 5.42 (s, 2H), 3.58-3.66 (m, 2H), 3.43-3.52 (m, 2H), 1.08 (t, J=8 Hz, 6H).

Step C:

Benzyl 6-chloro-2-fluoro-3-(N-(propylsulfonyl)propylsulfonamido)benzoate (5.4 g, 10.98 mmol) was dissolved in THF (100 mL) and 1M aqueous KOH (100 mL). This mixture was refluxed for 16 hours and then allowed to cool to room temperature. The mixture was then acidified to a pH of 2 with 2M aqueous HCl and extracted with EtOAc (2×). The extracts were washed with water, dried over sodium sulfate and concentrated to a solid that was triturated with hexanes/ether to give 6-chloro-2-fluoro-3-(propylsulfonamido)benzoic acid (2.2 g, 68%) as a solid. ¹H NMR (DMSO-d₆, 400 MHz) δ 9.93 (s, 1H), 7.49 (t, J=8 Hz, 1H), 7.38 (dd, J=8, 2 Hz, 1H), 3.11-3.16 (m, 2H), 1.68-1.78 (m, 2H), 0.97 (t, J=8 Hz, 3H).

### Example G

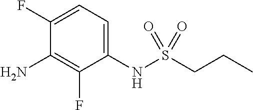 [SMILES: CCCS(=O)(=O)Nc1ccc(F)c(N)c1F]

### N-(3-Amino-2,4-difluorophenyl)propane-1-sulfonamide

To a solution of 2,6-difluoro-3-(propylsulfonamido)benzoic acid (4.078 g, 14.6 mmol) in THF (60 mL) was added triethylamine (4.68 mL, 33.59 mmol) and diphenylphosphonic azide (3.73 mL, 16.79 mmol). The reaction mixture was stirred at room temperature for 3 hours and then warmed to 80° C. for 2 hours. Water (10 mL) was added, and the mixture stirred at 80° C. for 15 hours. The reaction mixture was diluted with 300 mL of EtOAc, and the organic layer was washed with saturated aq. NaHCO₃ solution and brine. The solvent was removed under reduced pressure and the residual purified via silica gel column chromatography eluting with 30/70 EtOAc/hexane to obtain 2.03 g (55%) of the title compound. ¹H NMR (400 MHz, DMSO-d₆) δ 9.32 (s, 1H), 6.90-6.80 (m, 1H), 6.51 (td, J=8.7, 5.5 Hz, 1H), 5.28 (s, 2H), 3.05-2.96 (m, 2H), 1.82-1.64 (m, 2H), 1.01-0.90 (m, 3H). LC/MS: m/z 251.1 [M+1].

### Example H

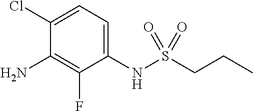 [SMILES: CCCS(=O)(=O)Nc1ccc(Cl)c(N)c1F]

### N-(3-Amino-4-chloro-2-fluorophenyl)propane-1-sulfonamide

To a solution of 6-chloro-2-fluoro-3-(propylsulfonamido)benzoic acid (1.70 g, 5.75 mmol) in THF (23 mL) was added triethylamine (1.84 mL, 13.2 mmol) and diphenylphosphonic azide (1.43 mL, 6.61 mmol). The reaction mixture was stirred at room temperature for 1 hour, warmed to 70° C. and stirred for 1 hour. Water (6 mL) was added, after which the reaction mixture was stirred again at 70° C. for 3 hours. The mixture was cooled to room temperature, ethyl acetate was added, and the layers were separated. The organic phase was dried with sodium sulfate, filtered and concentrated in vacuo. The crude product was purified by flash silica gel chromatography using 0-50% EtOAc/heptane gradient to afford N-(3-amino-4-chloro-2-fluorophenyl)propane-1-sulfonamide (1.01 g, 66%) as an off-white solid. ¹H NMR (500 MHz, DMSO-d₆) δ 9.54 (s, 1H), 7.02 (d, 1H), 6.58 (t, 1H), 5.50 (s, 2H), 3.09-2.95 (t, 2H), 1.81-1.64 (sx, 2H), 0.96 (t, 3H). LC/MS: m/z 267.1 [M+1].

### Example I

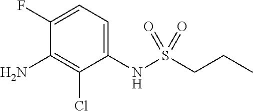 [SMILES: CCCS(=O)(=O)Nc1ccc(F)c(N)c1Cl]

### N-(3-Amino-2-chloro-4-fluorophenyl)propane-1-sulfonamide

The compound was prepared using the procedure described in Example G using 2-chloro-6-fluoro-3-(propylsulfonamido)benzoic acid instead 2,6-difluoro-3-(propyl-sulfonamido)benzoic acid as starting material. ¹H NMR (400 MHz, DMSO-d₆) δ 9.20 (s, 1H), 7.28-6.99 (m, 1H), 6.63 (td, J=8.7, 5.5 Hz, 1H), 5.45 (s, 2H), 3.07-2.99 (m, 2H), 1.88-1.69 (m, 2H), 1.03-0.95 (m, 3H). LC/MS: m/z 267.1 [M+1].

### Example J

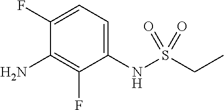

### N-(3-amino-2,4-difluorophenyl)ethanesulfonamide

To a solution of 2,6-difluoro-3-(propylsulfonamido)benzoic acid (6.643 g, 25.1 mmol) in THF (50 mL) was added triethylamine (8.02 mL, 57.61 mmol) and diphenylphosphonic azide (6.21 mL, 28.81 mmol). The reaction mixture was stirred at room temperature for 3 hours and then warmed to 80° C. for 2 hours. Water (15 mL, 830 mmol) was added and the mixture stirred at 80° C. for 15 hours. The reaction mixture was diluted with 500 mL of EtOAc, and the organic layer was washed with saturated aqueous NaHCO₃ solution and brine. The solvent was removed under reduced pressure and the residual purified via silica gel column chromatography eluting with EtOAc/hexane (30/70) to obtain 3.03 g (50%) of the title compound. ¹H NMR (400 MHz, DMSO-d₆) δ 9.37 (d, J=29.6 Hz, 1H), 6.86 (ddd, J=10.7, 9.1, 1.9 Hz, 1H), 6.52 (td, J=8.7, 5.5 Hz, 1H), 5.28 (s, 2H), 3.03 (q, J=7.3 Hz, 2H), 1.25 (td, J=7.3, 2.5 Hz, 3H). LC/MS: m/z 237.1 [M+1].

### Example K

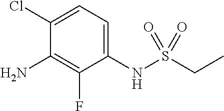

### N-(3-Amino-4-chloro-2-fluorophenyl)ethanesulfonamide

To a solution of 6-chloro-3-(ethylsulfonamido)-2-fluorobenzoic acid (8.00 g, 28.4 mmol) in THF (115 mL) was added triethylamine (9.10 mL, 65.3 mmol) and diphenylphosphonic azide (7.04 mL, 32.7 mmol). The reaction mixture was stirred at room temperature for 4 hours, warmed to 70° C. and stirred for 2 hours. Water (27 mL) was then added, after which the reaction mixture was stirred again at 70° C. for 16 hours. The mixture was cooled to room temperature, ethyl acetate and a saturated solution of NaHCO₃ were added, and the layers were separated. The organic phase was dried with sodium sulfate, filtered and concentrated in vacuo. The crude product was purified by flash silica gel chromatography using 30-50% EtOAc/heptane gradient to afford N-(3-amino-4-chloro-2-fluorophenyl)ethanesulfonamide (4.24 g, 59%) as a solid. ¹H NMR (500 MHz, DMSO-d₆) δ 9.48 (s, 1H), 7.02 (dd, J=8.8, 1.7 Hz, 1H), 6.59 (t, J=8.3 Hz, 1H), 5.44 (s, 2H), 3.07 (q, J=7.3 Hz, 2H), 1.24 (t, J=7.3 Hz, 3H). LC/MS: m/z 253.2 [M+1].

### Example L

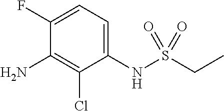

### N-(3-Amino-2-chloro-4-fluorophenyl)ethanesulfonamide

2-Chloro-6-fluoro-3-(ethylsulfonamido)benzoic acid (3.3 g, 12.0 mmol) was treated with thionyl chloride (21.0 mL, 0.29 mmol) and heated at reflux for 15 hours. The reaction mixture was concentrated and then azeotrophed with toluene (2×20 mL). The residue was treated with a solution of sodium azide (3.1 g, 48.0 mmol) dissolved in water (20 mL) and acetone (20 mL). After stirring at room temperature for 1 hour, the intermediate acyl azide was extracted into ethyl acetate (2×25 mL), dried with magnesium sulfate and concentrated. The residue was dissolved in dioxane (40 mL) and water (5 mL) and heated to reflux for 3 hours. After cooling to room temperature, the product was extracted into methylene chloride (2×25 mL), dried with magnesium sulfate and concentrated. The residue was purified by flash silica gel chromatography (2-30% isopropanol in methylene chloride) to afford N-(3-amino-2-chloro-4-fluorophenyl)ethanesulfonamide. (2.0 g, 66%) ¹H NMR (400 MHz, DMSO-d₆) δ 9.15 (s, 1H), 7.02 (dd, J=10.7, 8.8 Hz, 1H), 6.64 (dd, J=8.8, 5.1 Hz, 1H), 5.45 (s, 2H), 3.06 (q, J=7.3 Hz, 2H), 0.96 (t, J=7.3 Hz, 3H). LC/MS: m/z 253.0 [M+1]. LC/MS: m/z 253.0 [M+1].

### Example M

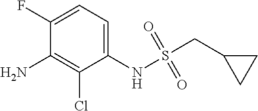 [SMILES: Nc1c(F)ccc(NS(=O)(=O)CC2CC2)c1Cl]

### N-(3-Amino-2-chloro-4-fluorophenyl)-1-cyclopropylmethanesulfonamide

Step A:

To a solution of methyl 3-amino-2-chloro-6-fluorobenzoate (2.97 g, 14.6 mmol) in THF (26 mL) and triethylamine (6.10 mL, 43.8 mmol) at 0° C. was added cyclopropylmethanesulfonyl chloride (4.74 g, 30.6 mmol) dropwise. The reaction mixture was stirred at 0° C. for 90 minutes, after which 8N NaOH (18.2 mL, 140 mmol) was added. The reaction mixture was then warmed up at 40° C. and stirred for 16 hours. The volatiles were removed in vacuo and the mixture acidified with concentrated HCl at 0° C. to pH 1. The acidified mixture was extracted with ethyl acetate twice. The organic phases were combined, dried with sodium sulfate, filtered and concentrated in vacuo to obtain crude 2-chloro-3-(cyclopropylmethylsulfonamido)-6-fluorobenzoic acid, which was used directly in the next step without further purification.

Step B:

To a solution of 2-chloro-3-(cyclopropylmethylsulfonamido)-6-fluorobenzoic acid (4.11 g, 13.4 mmol) in 1,4-dioxane (30 mL) was added triethylamine (2.05 mL, 14.7 mmol), followed by diphenylphosphonic azide (3.12 mL, 14.0 mmol) at room temperature. The reaction was stirred at room temperature for 4 hours and the resulting mixture added dropwise, via an addition funnel, over 15 minutes in a round-bottom flask containing 1,4-dioxane (16 mL) and water (1.20 mL, 66.8 mmol) at 95° C. The reaction mixture was stirred at this temperature for 16 hours. The reaction mixture was concentrated to half the volume in vacuo and diluted with ethyl acetate and a saturated solution of NaHCO₃. The layers were separated and the aqueous layer extracted twice with ethyl acetate. The organic phases were combined, dried with sodium sulfate, filtered and concentrated in vacuo. The crude product was purified by flash chromatography to afford N-(3-amino-2-chloro-4-fluorophenyl)-1-cyclopropylmethanesulfonamide (2.05 g, 55%). ¹H NMR (500 MHz, DMSO) δ 9.07 (s, 1H), 7.01 (dd, J=10.7, 8.9 Hz, 1H), 6.66 (dd, J=8.8, 5.1 Hz, 1H), 5.43 (s, 2H), 3.04 (d, J=7.1 Hz, 2H), 1.12-0.99 (m, 1H), 0.59-0.52 (m, 2H), 0.36-0.30 (m, 2H).

### Example N

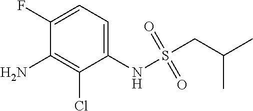 [SMILES: CC(C)CS(=O)(=O)Nc1ccc(F)c(N)c1Cl]

### N-(3-Amino-2-chloro-4-fluorophenyl)-2-methylpropane-1-sulfonamide

Step A:

To a solution of methyl 3-amino-2-chloro-6-fluorobenzoate (2.97 g, 14.6 mmol) in THF (20 mL) and triethylamine (6.10 mL, 43.8 mmol) at 0° C. was added 2-methylpropane-1-sulfonyl chloride (4.80 g, 30.6 mmol) dropwise. The reaction mixture was stirred at 0° C. for 90 minutes, after which 8N aqueous NaOH (18.2 mL, 140 mmol) was added. The reaction mixture was heated with stirring at 40° C. for 16 hours. The volatiles were then removed in vacuo and the mixture acidified with concentrated HCl at 0° C. to pH 1. The acidified mixture was extracted with ethyl acetate twice. The organic phases were combined, dried with sodium sulfate, filtered and concentrated in vacuo to obtain crude 2-chloro-6-fluoro-3-(2-methylpropylsulfonamido)benzoic acid, which was used directly in the next step without further purification.

Step B:

N-(3-Amino-2-chloro-4-fluorophenyl)-2-methylpropane-1-sulfonamide was prepared according to the general procedure for Example M (step B), substituting 2-chloro-6-fluoro-3-(2-methylpropylsulfonamido)benzoic acid for 2-chloro-3-(cyclopropylmethylsulfonamido)-6-fluorobenzoic acid. m/z (ES-MS) M+1=281.2. ¹H NMR (500 MHz, DMSO) δ 9.14 (s, 1H), 7.02 (dd, J=10.7, 8.9 Hz, 1H), 6.64 (dd, J=8.8, 5.1 Hz, 1H), 5.44 (s, 2H), 2.96 (d, J=6.4 Hz, 2H), 2.20-2.10 (m, 1H), 1.01 (d, J=6.7 Hz, 6H).

### Example O

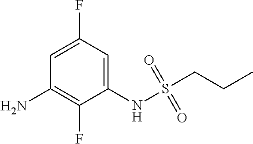 [SMILES: CCCS(=O)(=O)Nc1cc(F)cc(N)c1F]

### N-(3-Amino-2,5-difluorophenyl)propane-1-sulfonamide

To a solution of 2,5-difluorobenzene-1,3-diamine (2.00 g, 13.9 mmol) (described in E.P. Pat. Appl. Publication No. 0,415,595) in THF (40 mL) and pyridine (1.571 mL, 19.43 mmol) was added propane-1-sulfonyl chloride (1.867 mL, 16.65 mmol) at 0° C. The reaction mixture was stirred at 50° C. for 90 minutes and DCM and a saturated solution of NaHCO₃ were then added. The layers were separated and the aqueous layer was extracted twice with DCM. The organic layers were combined, dried with sodium sulfate, filtered and concentrated in vacuo. The crude mixture was re-submitted to exact same reaction conditions, and the reaction was stirred at 55° C. for 16 hours, and ethyl acetate and a saturated solution of NaHCO₃ were then added. The layers were separated and the aqueous layer extracted twice with ethyl acetate. The organic layers were combined, dried with sodium sulfate, filtered and concentrated in vacuo. The crude product was purified by flash chromatography to afford N-(3-amino-2,5-difluorophenyl)propane-1-sulfonamide (485 mg, 14%). ¹H NMR (400 MHz, DMSO-d₆) δ 9.57 (s, 1H), 6.39-6.23 (m, 2H), 5.55 (s, 2H), 3.12-2.99 (m, 2H), 1.77-1.66 (m, 2H), 0.96 (t, J=7.3 Hz, 3H). m/z (ES-MS) M+1=251.2.

### Example P

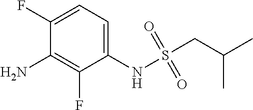 [SMILES: CC(C)CS(=O)(=O)Nc1ccc(F)c(N)c1F]

### N-(3-Amino-2,4-difluorophenyl)-2-methylpropane-1-sulfonamide

Step A:

2,6-Difluoro-3-(2-methylpropylsulfonamido)benzoic acid was prepared according to the general procedure for Example N (step A), substituting methyl 3-amino-2,6-difluorobenzoate for methyl 3-amino-2-chloro-6-fluorobenzoate.

Step B:

N-(3-Amino-2,4-difluorophenyl)-2-methylpropane-1-sulfonamide was prepared according to the general procedure for Example M (step B), substituting 2,6-difluoro-3-(2-methylpropylsulfonamido)benzoic acid for 2-chloro-3-(cyclopropylmethylsulfonamido)-6-fluorobenzoic acid. m/z (ES-MS) M+1=265.2. ¹H NMR (400 MHz, DMSO) δ 9.36 (s, 1H), 6.86 (t, J=9.8 Hz, 1H), 6.55-6.47 (m, 1H), 5.32 (s, 2H), 2.93 (d, J=6.4 Hz, 2H), 2.21-2.10 m, 1H), 1.01 (d, J=6.7 Hz, 6H).

### Example Q

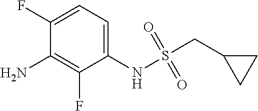 [SMILES: Nc1c(F)ccc(NS(=O)(=O)CC2CC2)c1F]

### N-(3-Amino-2,4-difluorophenyl)-1-cyclopropylmethanesulfonamide

Step A:

3-(Cyclopropylmethylsulfonamido)-2,6-difluorobenzoic acid was prepared according to the general procedure for Example M (step A), substituting methyl 3-amino-2,6-difluorobenzoate for methyl 3-amino-2-chloro-6-fluorobenzoate.

Step B:

N-(3-Amino-2,4-difluorophenyl)-1-cyclopropylmethanesulfonamide was prepared according to the general procedure for Example M (step B), substituting 3-(cyclopropylmethylsulfonamido)-2,6-difluorobenzoic acid for 2-chloro-3-(cyclopropylmethyl-sulfonamido)-6-fluorobenzoic acid. m/z (ES-MS) M+1=263.2. ¹H NMR (400 MHz, DMSO) δ 9.37 (s, 1H), 6.84 (t, J=9.8 Hz, 1H), 6.57-6.50 (m, 1H), 5.30 (s, 2H), 3.01 (d, J=7.1 Hz, 2H), 1.11-0.98 (m, 1H), 0.59-0.51 (m, 2H), 0.35-0.27 (m, 2H).

### Example R

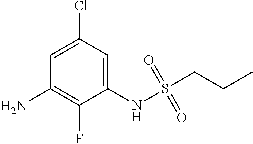 [SMILES: CCCS(=O)(=O)Nc1cc(Cl)cc(N)c1F]

### N-(3-Amino-5-chloro-2-fluorophenyl)propane-1-sulfonamide

Step A:

To a solution of methyl 5-chloro-2-fluorobenzoate (16.0 g, 84.8 mmol) in sulfuric acid (100 mL) at 0° C. was added fuming nitric acid (4.98 mL, 119 mmol). The reaction mixture was stirred at room temperature for 3 hours, poured into ice/water and the resulting precipitate was filtered. The obtained solid was purified by flash chromatography to afford methyl 5-chloro-2-fluoro-3-nitrobenzoate (6.78 g, 30%).

Step B:

A round-bottom flask was charged with 5-chloro-2-fluoro-3-nitrobenzoate (6.78 g, 29.0 mmol), iron (16.2 g, 290 mmol), ammonium chloride (5.43 g, 102 mmol), ethanol (100 mL) and water (30 mL). The reaction mixture was stirred at 85° C. for 2 hours, then cooled to room temperature. The mixture was diluted with ethyl acetate and a saturated solution of NaHCO₃, and the layers were separated. The aqueous layer was extracted twice with ethyl acetate. The organic layers were combined, dried with sodium sulfate, filtered and concentrated in vacuo. The crude product was purified by flash chromatography to afford methyl 3-amino-5-chloro-2-fluorobenzoate (3.7 g, 63%).

Step C:

To a solution of methyl 3-amino-5-chloro-2-fluorobenzoate (2.7097 g, 13.3 mmol) in THF (25 mL) and triethylamine (5.54 mL, 39.8 mmol) at 0° C. was added propane-1-sulfonyl chloride (3.12 mL, 27.8 mmol) dropwise. The reaction mixture was stirred at 0° C. for 90 minutes, after which 8N aqueous NaOH (16.6 mL, 130 mmol) was added. The reaction mixture was heated with stirring at 40° C. for 16 hours. The volatiles were removed in vacuo, and the mixture was acidified with concentrated HCl at 0° C. to pH 1. The acidified mixture was extracted with ethyl acetate twice. The organic layers were combined, dried with sodium sulfate, filtered and concentrated in vacuo to obtain crude 5-chloro-2-fluoro-3-(propylsulfonamido)benzoic acid, which was used in the next step without further purification.

Step D:

N-(3-Amino-5-chloro-2-fluorophenyl)propane-1-sulfonamide was prepared according to the general procedure for Example M (step B), substituting 5-chloro-2-fluoro-3-(propylsulfonamido)benzoic acid for 2-chloro-3-(cyclopropylmethylsulfonamido)-6-fluorobenzoic acid. m/z (ES-MS) M+1=267.0. ¹H NMR (400 MHz, DMSO-d₆) δ 9.58 (s, 1H), 6.59 (dd, J=7.1, 2.6 Hz, 1H), 6.53 (dd, J=5.9, 2.6 Hz, 1H), 5.56 (s, 2H), 3.11-3.03 (m, 2H), 1.78-1.65 (m, 2H), 0.97 (t, J=7.4 Hz, 3H).

### Example S

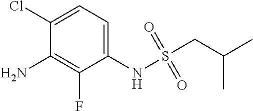 [SMILES: CC(C)CS(=O)(=O)Nc1ccc(Cl)c(N)c1F]

### N-(3-Amino-4-chloro-2-fluorophenyl)-2-methylpropane-1-sulfonamide

Step A:

Benzyl 6-chloro-2-fluoro-3-(N-(isobutylsulfonyl)-2-methylpropyl-sulfonamido)benzoate was prepared according to the general procedure for Example F (step B), substituting 2-methylpropane-1-sulfonyl chloride for propane-1-sulfonyl chloride.

Step B:

6-Chloro-2-fluoro-3-(2-methylpropylsulfonamido)benzoic acid was prepared according to the general procedure for Example F (step C) substituting benzyl 6-chloro-2-fluoro-3-(N-(isobutylsulfonyl)-2-methylpropylsulfonamido)benzoate for benzyl 6-chloro-2-fluoro-3-(N-(propylsulfonyl)propylsulfonamido)benzoate.

Step C:

N-(3-Amino-4-chloro-2-fluorophenyl)-2-methylpropane-1-sulfonamide was prepared according to the general procedure for Example M (step B), substituting 6-chloro-2-fluoro-3-(2-methylpropylsulfonamido)benzoic acid for 2-chloro-3-(cyclopropylmethylsulfonamido)-6-fluorobenzoic acid. m/z (ES-MS) M+1=281.2. ¹H NMR (400 MHz, DMSO) δ 9.50 (s, 1H), 7.02 (dd, J=8.8, 1.8 Hz, 1H), 6.62-6.54 (m, 1H), 5.45 (s, 2H), 2.97 (d, J=6.4 Hz, 2H), 2.21-2.10 (m, 1H), 1.01 (d, J=6.7 Hz, 6H).

### Example T

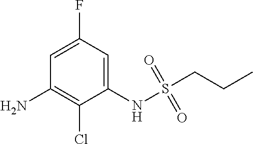 [SMILES: CCCS(=O)(=O)Nc1cc(F)cc(N)c1Cl]

### N-(3-Amino-2-chloro-5-fluorophenyl)propane-1-sulfonamide

Step A:

To a solution of 2-chloro-5-fluorobenzene-1,3-diamine (1.01 g, 6.29 mmol; 70% purity) (described in U.S. Pat. Publication No. 2006/0258888) in DCM (30 mL) and triethylamine (1.93 mL, 13.8 mmol) was added propane-1-sulfonyl chloride (1.41 mL, 12.6 mmol) at 0° C. The reaction mixture was stirred at room temperature for 1 hour. An aqueous saturated solution of NaHCO₃ and ethyl acetate were added, and the layers were separated. The aqueous layer was extracted twice with ethyl acetate. The organic phases were combined, dried with sodium sulfate, filtered and concentrated in vacuo. The crude mixture was dissolved in tetrahydrofuran (15 mL) and methanol (5 mL), and 1.0 M of sodium hydroxide in water (6.3 mL) was added. The reaction mixture was stirred at room temperature for 30 minutes. An aqueous saturated solution of NaHCO₃ and ethyl acetate were added, and the layers were separated. The aqueous layer was extracted twice with ethyl acetate. The organic layers were combined, dried with sodium sulfate, filtered and concentrated in vacuo. The crude product was purified by flash chromatography to afford N-(3-amino-2-chloro-5-fluorophenyl)propane-1-sulfonamide (0.17 g, 7%). m/z (ES-MS) M+1=281.2.

### Example U

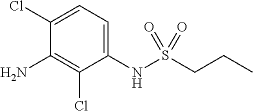 [SMILES: CCCS(=O)(=O)Nc1ccc(Cl)c(N)c1Cl]

### N-(3-Amino-2,4-dichlorophenyl)propane-1-sulfonamide

Step A:

2,6-Dichloro-3-nitrobenzoic acid (2.13 g, 9.03 mmol) was dissolved in 2:1 THF:saturated aqueous NH₄Cl and cooled to 0° C. The mixture was treated with zinc (11.8 g, 181 mmol) and then allowed to warm to ambient temperature and stirred for 24 hours. The reaction mixture was filtered through GF/F paper while rinsing with THF. The mixture was acidified to a pH of 1 using 1.0 M HCl and extracted with 15% 2-propanol/DCM (3×). The extracts were washed with water and brine, dried over sodium sulfate and concentrated to afford 3-amino-2,6-dichlorobenzoic acid (1.40 g, 6.82 mmol, 75.5% yield). MS (APCI-neg) m/z=203.6 (M−H).

Step B:

3-Amino-2,6-dichlorobenzoic acid (1.40 g, 6.82 mmol) was dissolved in dry dichloromethane (66.7 mL). Triethylamine (4.09 mL, 29.4 mmol) was added, and the mixture was chilled to 0° C. Propane-1-sulfonyl chloride (2.48 mL, 22 mmol) was then added using a syringe. When the addition was complete, the mixture was allowed to warm to ambient temperature and stirred for 1 hour. The mixture was concentrated in vacuo and diluted with diethyl ether. The mixture was washed with 0.25 M NaOH (80 mL) and the aqueous layer acidified to a pH of 1 using 1.0 M HCl. The aqueous layer was extracted with 15% 2-propanol:DCM (2×300 mL). The organic layer was collected, dried over sodium sulfate, and concentrated to afford 2,6-dichloro-3-(propylsulfonamido)benzoic acid (1.55 g, 4.96 mmol, 74.4% yield). ¹H NMR (400 MHz, DMSO-d₆) δ 9.77-9.75 (s, 1H), 7.84-7.80 (d, 1H), 7.71-7.68 (d, 1H), 3.82-3.72 (m, 2H), 1.89-1.70 (m, 2H), 1.05-1.03 (m, 3H).

Step C:

To a solution of 2,6-dichloro-3-(propylsulfonamido)benzoic acid (2.788 g, 8.93 mmol in THF (40 mL) was added triethylamine (2.863 mL, 20.5 mmol) and diphenylphosphonic azide (2.282 mL, 10.2 mmol). The reaction mixture was stirred for 6 hours at room temperature. Water (8 mL, 400 mmol) was added, and the reaction mixture was heated under reflux overnight. Ethyl acetate (300 mL) was added, followed by washing with saturated aqueous NaHCO₃ solution and brine. The solvent was removed under reduced pressure and the crude product purified via silica gel flash chromatography using ethyl acetate/hexane (1:1) as eluent to yield 834 mg (33%) of N-(3-amino-2,4-dichlorophenyl)propane-1-sulfonamide. ¹H NMR (500 MHz, DMSO-d₆) δ 9.24 (s, 1H), 7.20 (d, J=8.7 Hz, 1H), 6.71 (d, J=8.7 Hz, 1H), 5.55 (s, 2H), 3.13-2.92 (m, 2H), 1.73 (dd, J=15.2, 7.6 Hz, 2H), 0.96 (t, J=7.4 Hz, 3H). LC-MS [M+1] m/z 284.1

### Example V

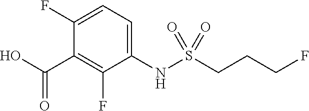 [SMILES: O=C(O)c1c(F)ccc(NS(=O)(=O)CCCF)c1F]

### 2,6-Difluoro-3-(3-fluoropropylsulfonamido)benzoic acid

Step A:

Into a 3000-mL 4-necked round-bottom flask was placed a solution of methyl 3-amino-2,6-difluorobenzoate (120 g, 609.63 mmol, 1.00 equiv, 95%) in dichloromethane (1800 mL) and pyridine (152 g, 1.92 mol, 3.16 equiv) followed by the addition of 3-fluoropropane-1-sulfonyl chloride (103 g, 643.75 mmol, 1.06 equiv) dropwise with stirring at 8° C. After stirred overnight at 8° C., the resulting mixture was washed with 2×400 mL of 5N HCl and 2×400 ml of brine. The organic layer was dried over anhydrous sodium sulfate and concentrated under vacuum to afford 150 g (75%) of methyl 2,6-difluoro-3-(3-fluoropropylsulfonamido)benzoate as a lavender colored solid.

Step B:

A solution of methyl 2,6-difluoro-3-(3-fluoropropylsulfonamido)benzoate (150 g, 458.2 mmol, 1.00 equiv, 95%) in tetrahydrofuran (750 mL) and KOH (aq. 2N, 750 mL) was stirred at 50° C. in an oil bath for 3.5 h, cooled and concentrated under vacuum. The residual solution was adjusted to pH 2-3 with 6N HCl and extracted with 3×1000 mL of ethyl acetate. The combined organic layers were dried over anhydrous sodium sulfate and concentrated under vacuum to afford 140 g (crude) of 2,6-difluoro-3-(3-fluoropropylsulfonamido)benzoic acid as a lavender colored solid. ¹H NMR (500 MHz, DMSO-d₆) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H). MS m/z 296.1 [M−1].

### Example W

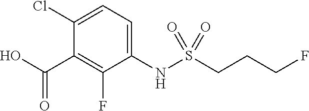 [SMILES: O=C(O)c1c(Cl)ccc(NS(=O)(=O)CCCF)c1F]

### 6-Chloro-2-fluoro-3-(3-fluoropropylsulfonamido)benzoic acid

Step A:

Into a 5000-mL 4-necked round-bottom flask was placed a solution of benzyl 3-amino-6-chloro-2-fluorobenzoate (200 g, 714.29 mmol, 1.00 equiv) in dichloromethane (2000 mL) and triethylamine (216 g, 2.14 mol, 3.00 equiv) followed by the addition of a solution of 3-fluoropropane-1-sulfonyl chloride (227 g, 1.42 mol, 2.00 equiv) in dichloromethane (300 mL) dropwise with stirring at 8° C. over 60 min. After stirred at room temperature for 3 h, the resulting mixture was washed with 500 mL of 5N HCl and 2×500 mL of water. The organic layer was dried over anhydrous sodium sulfate and concentrated under vacuum to afford 360 g (91%) of benzyl 6-chloro-2-fluoro-3-(3-fluoro-N-(3-fluoropropyl-sulfonyl)propylsulfonamido)benzoate as a brown oil.

Step B:

A solution of benzyl 6-chloro-2-fluoro-3-(3-fluoro-N-(3-fluoropropyl-sulfonyl)propylsulfonamido)benzoate (360 g, 647.73 mmol, 1.00 equiv, 95%) in tetrahydrofuran (1800 mL) and KOH (2M, 1680 mL) was stirred at 50° C. for 12 h. The resulting mixture was cooled and concentrated under vacuum to remove most of THF. The residual solution was washed with 3×500 mL of EtOAc. The aqueous layer was adjusted to pH 2-3 with HCl (6M). The resulting solution was extracted with 4×500 mL of ethyl acetate. The combined organic layers were dried over anhydrous sodium sulfate and concentrated under vacuum to afford 190 g (89%) of 6-chloro-2-fluoro-3-(3-fluoropropylsulfonamido)benzoic acid as a pink solid. ¹H NMR (400 MHz, DMSO-d₆) δ 9.65 (br s, 1H), 7.03 (m, 1H), 6.58 (m, 1H), 4.59 (m, 1H), 4.47 (m, 1H), 3.18 (m, 2H), 2.22-2.02 (m, 2H). MS m/z 312.1, 314.1 [M−1].

### Example X

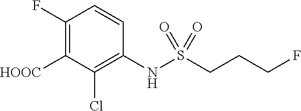 [SMILES: O=C(O)c1c(F)ccc(NS(=O)(=O)CCCF)c1Cl]

### 2-Chloro-6-fluoro-3-(3 fluoropropylsulfonamido)benzoic acid

Step A:

Into a 2000-mL 3-necked round-bottom flask was placed a solution of methyl 3-amino-2-chloro-6-fluorobenzoate (50 g, 243.84 mmol, 1.00 equiv, 99%) in dichloromethane (900 mL) followed by the addition of triethylamine (75 g, 726.28 mmol, 3.00 equiv, 98%) dropwise with stirring at 0° C. To this was added a solution of 3-fluoropropane-1-sulfonyl chloride (55.6 g, 344.02 mmol, 1.30 equiv, 99%) in dichloromethane (100 mL) dropwise with stirring at −15° C. After stirring overnight at room temperature, the resulting solution was diluted with 500 mL of DCM, washed with 2×500 mL of water and 5×500 mL of HCl (4N) to remove starting material. The organic layer, containing methyl 2-chloro-6-fluoro-3-(3-fluoropropylsulfonamido)benzoate and methyl 2-chloro-6-fluoro-3-(3-fluoro-N-(3-fluoropropylsulfonyl)propylsulfonamido)benzoate as a contamination, was washed with 2×500 mL of brine, dried over anhydrous sodium sulfate and concentrated under vacuum. This resulted in 90 g crude mixture as a yellow oil which was used in the next step.

Step B:

Into a 1000-mL round-bottom flask were placed a solution of crude mixture from last step in tetrahydrofuran (250 mL) and a solution of potassium hydroxide (60 g, 1.05 mol, 3.00 equiv, 98%) in water (250 mL). The resulting solution was refluxed for 1 h in an oil bath, cooled to room temperature with a water/ice bath, concentrated under vacuum, diluted with 100 mL of H₂O, and washed with 3×500 mL of ethyl acetate. The aqueous layer was adjusted to pH 1 with HCl (2 mol/L). The resulting solution was extracted with 5×200 mL of ethyl acetate. The combined organic layers were washed with 1×500 mL of brine, dried over anhydrous sodium sulfate and concentrated under vacuum. The residue was washed with 1×200 ml of hexane and dried to afford 60 g (78%, two steps) of 2-chloro-6-fluoro-3-(3-fluoropropylsulfonamido)benzoic acid as a yellowish solid. ¹H NMR (400 MHz, MeOH-d₄) δ 7.63 (m, 1H), 7.19 (m, 1H), 4.56 (m, 1H), 4.44 (m, 1H), 3.21 (m, 2H), 2.25-2.12 (m, 2H). MS m/z 312.1, 314.1 [M−1].

### Example Y

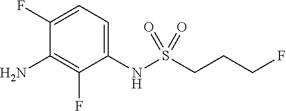 [SMILES: Nc1c(F)ccc(NS(=O)(=O)CCCF)c1F]

### N-(3-Amino-2,4-difluorophenyl)-3-fluoropropane-1-sulfonamide

Into a 3000-mL 4-necked round-bottom flask were placed a solution of 2,6-difluoro-3-(3-fluoropropylsulfonamido)benzoic acid (150 g, 479.80 mmol, 1.00 equiv, 95%) in N,N-dimethylformamide (1200 mL), TEA (153 g, 1.51 mol, 3.00 equiv) and DPPA (208.5 g, 758.18 mmol, 1.50 equiv). The resulting solution was stirred at 6° C. for 2 h followed by the addition of water (364 mL, 40.00 equiv). The resulting solution was stirred at 80° C. in an oil bath for 1.5 h, diluted with 3 L of H₂O, and extracted with 3×1 L of ethyl acetate. The combined organic layers were washed with 3×1 L of H₂O, dried over anhydrous sodium sulfate and concentrated under vacuum. The residue was applied onto a silica gel column eluted with ethyl acetate/petroleum ether (1:2) to afford 74.74 g (58%) of N-(3-amino-2,4-difluorophenyl)-3-fluoropropane-1-sulfonamide as a brown solid. ¹H NMR (400 MHz, CDCl₃, ppm): 2.265 (2H, m), 3.252 (2H, m), 3.805 (2H, br), 4.494 (1H, t), 4.611 (1H, t), 6.274 (1H, s), 6.842 (2H, m). LC-MS (ES, m/z): 268 [M+H]⁺.

### Example Z

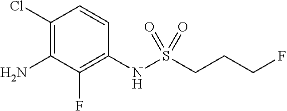 [SMILES: Nc1c(Cl)ccc(NS(=O)(=O)CCCF)c1F]

### N-(3-Amino-4-chloro-2-fluorophenyl)-3-fluoropropane-1-sulfonamide

Into a 3000-mL 3-necked round-bottom flask was placed a solution of 6-chloro-2-fluoro-3-(3-fluoropropylsulfonamido)benzoic acid (190 g, 574.84 mmol, 1.00 equiv, 95%) in N,N-dimethylformamide (1500 mL) and triethylamine (184 g, 1.82 mol, 3.00 equiv) followed by the addition of DPPA (250 g, 909.09 mmol, 1.50 equiv) dropwise with stirring at 5° C. over 10 min. After stirred at 5° C. for 2 h, to the reaction mixture was added water (500 mL). The resulting solution was stirred at 80° C. in an oil bath for an additional 2 h, cooled and diluted with 2000 mL of EtOAc. The organic layer was washed with 4×1000 mL of brine, dried over anhydrous sodium sulfate and concentrated under vacuum. The residue was applied onto a silica gel column eluted with ethyl acetate/petroleum ether (1:3) to afford 76 g (46%) of N-(3-amino-4-chloro-2-fluorophenyl)-3-fluoropropane-1-sulfonamide as a white solid. ¹H-NMR (400 MHz, CDCl₃, ppm): 7.04-7.06 (1H, m), 6.91-6.87 (1H, t), 6.39 (1H, s), 4.62-4.59 (1H, t), 4.40-4.57 (1H, t), 4.15 (1H, br), 3.27-3.24 (2H, t), 2.30-2.16 (2H, m). LC-MS (ES, m/z): 283 [M−H]⁻.

### Example AA

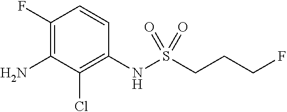 [SMILES: Nc1c(F)ccc(NS(=O)(=O)CCCF)c1Cl]

### N-(3-Amino-2-chloro-4-fluorophenyl)-3-fluoropropane-1-sulfonamide

Into three 1000-mL 3-necked round-bottom flask, purged and maintained with an inert atmosphere of nitrogen, was placed a solution of 2-chloro-6-fluoro-3-(3-fluoropropylsulfonamido)benzoic acid (147 g, 422.68 mmol, 1.00 equiv, 90%) in N,N-dimethylformamide (1170 mL) followed by the addition of triethylamine (142 g, 1.38 mol, 3.00 equiv, 98%) dropwise with stirring at 0-5° C. To this was added diphenylphosphoryl azide (200 g, 712.73 mmol, 1.50 equiv, 98%) dropwise with stirring at 0° C. The resulting solution was stirred at 25° C. for 4 h. The reaction mixture was diluted with water (340 mL). The resulting solution was stirred at 80° C. in an oil bath overnight, cooled to room temperature and concentrated under vacuum. The residual solution was diluted with 1500 mL of DCM and washed with 4×1000 mL of saturated sodium bicarbonate solution and 1×1000 mL of brine. The organic layer was dried over anhydrous sodium sulfate and concentrated under vacuum. The residue was applied onto a silica gel column eluted with ethyl acetate/petroleum ether (1:4) to afford 50.3 g (41%) of N-(3-amino-2-chloro-4-fluorophenyl)-3-fluoropropane-1-sulfonamide as a white solid. ¹H NMR (400 MHz, DMSO-d₆, ppm): δ9.84 (1H, s), 7.06-7.02 (1H, d), 6.65-6.62 (1H, d), 5.53 (2H, s), 4.62-4.59 (1H, m), 4.50-4.47 (1H, m), 3.18-3.15 (2H, m), 2.17-2.04 (2H, m). LC-MS (ES, m/z): 285 [M+H]⁺.

### Example AB

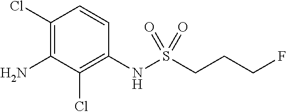 [SMILES: Nc1c(Cl)ccc(NS(=O)(=O)CCCF)c1Cl]

### N-(3-Amino-2,4-dichlorophenyl)-3-fluoropropane-1-sulfonamide

Step A:

To 3-amino-2,6-dichlorobenzoic acid (8.00 g, 38.8 mmol) in tetrahydrofuran (200 ml) at 0° C. was added dropwise triethylamine (29.8 mL, 214 mmol) followed by 3-fluoropropane-1-sulfonyl chloride (15.1 mL, 136 mmol). The reaction mixture was stirred at 50° C. for 16 hours and then cooled to room temperature. Water and dichloromethane were added. The biphasic layers were separated, and the aqueous layer was extracted twice with dichloromethane. The organic layers were combined, dried with sodium sulfate, filtered and concentrated in vacuo. The obtained oil was dissolved in tetrahydrofuran (107 mL) and 8 M NaOH (49 mL) was added dropwise at room temperature. The reaction mixture was heated at 50° C. Volatiles were removed in vacuo, and the reaction mixture was acidified with concentrated HCl at 0° C. to pH 1. The aqueous phase was then extracted twice with ethyl acetate. The organic layers were combined, dried with sodium sulfate, filtered and concentrated in vacuo. The crude product was purified by flash chromatography to afford 2,6-dichloro-3-(3-fluoropropylsulfonamido)benzoic acid (6.7 g, 44%).

Step B:

To a solution of 2,6-dichloro-3-(3-fluoropropylsulfonamido)benzoic acid (6.7 g, 20.0 mmol) in 1,4-dioxane (50 mL) was added triethylamine (3.11 mL, 22.3 mmol), followed by diphenylphosphonic azide (4.73 mL, 21.3 mmol) at room temperature. The reaction mixture was stirred at room temperature for 1 hour, then at 50° C. for 7 hours. The reaction mixture was subsequently added dropwise, via an addition funnel, over 15 minutes in a round-bottom flask containing 1,4-dioxane (24 ml) and water (1.83 mL, 101 mmol) at 95° C. The reaction was stirred at this temperature for 16 hours. The reaction mixture was concentrated in vacuo to half its volume and then diluted with ethyl acetate and a saturated solution of NaHCO₃. The biphasic layers were separated, and the aqueous layer was extracted twice with ethyl acetate. The organic layers were combined, dried with sodium sulfate, filtered and concentrated in vacuo. The crude product was purified by flash chromatography to afford N-(3-amino-2,4-dichlorophenyl)-3-fluoropropane-1-sulfonamide (3.06 g, 50%). ¹H NMR (400 MHz, DMSO) δ 9.45 (s, 1H), 7.23 (d, J=8.6 Hz, 1H), 6.70 (d, J=8.6 Hz, 1H), 5.61 (s, 2H), 4.59 (t, J=5.8 Hz, 1H), 4.48 (t, J=5.8 Hz, 1H), 3.25-3.15 (m, 2H), 2.20-2.01 (m, 2H). m/z (ES-MS) 301.2 (100%) [M+1].

### Example AC

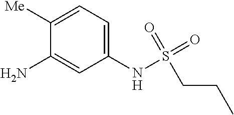

### (3-Amino-4-methylphenyl)propane-1-sulfonamide

Step A:

To 4-methyl-3-nitroaniline (1.0 g, 6.57 mmol) in DCM (30 mL) at 0° C. was added TEA (4.58 mL, 32.9 mmol), followed by propane-1-sulfonyl chloride (1.84 mL, 16.4 mmol). The solution was warmed to ambient temperature and stirred for 2 hours. The solution was diluted with aqueous bicarbonate (100 mL) and extracted with EtOAc (3×40 mL). The organics were dried over sodium sulfate, filtered and concentrated under reduced pressure to afford N-(4-methyl-3-nitrophenyl)-N-(propylsulfonyl)propane-1-sulfonamide (2.4 g, 100%).

Step B:

To N-(4-methyl-3-nitrophenyl)-N-(propylsulfonyl)propane-1-sulfonamide (2.4 g, 6.6 mmol) in 4:1 THF:MeOH (75 mL) was added 2 M NaOH (16 mL, 33 mmol). The solution was warmed to 50° C. for 3 hours. The cooled solution was concentrated under reduced pressure, and the residue was diluted with aqueous ammonium chloride (100 mL) and extracted with EtOAc (3×40 mL). The combined organics were washed with aqueous bicarbonate (2×50 mL), then dried over sodium sulfate, filtered, and concentrated under reduced pressure to afford N-(4-methyl-3-nitrophenyl)propane-1-sulfonamide (1.6 g, 94%).

Step C:

To N-(4-methyl-3-nitrophenyl)propane-1-sulfonamide (1.6 g, 6.19 mmol) in EtOH (30 mL) was added 10% Pd/C (1.32 g, 1.24 mmol). The suspension was stirred under a balloon of hydrogen at ambient temperature for 16 hours. The suspension was filtered and concentrated to afford (3-amino-4-methylphenyl)propane-1-sulfonamide (1.38 g, 97.6%). ¹H NMR (400 MHz, CD₃OD) δ 6.96-6.99 (m, 1H), 6.63-6.65 (m, 1H), 6.45-6.49 (m, 1H), 6.38 (br s, 1H), 3.02-3.07 (m, 2H), 2.13 (s, 3H), 1.75-1.90 (m, 2H), 0.98-1.04 (m, 3H). LC/MS: m/z 229.1 [M+1].

### Example AD

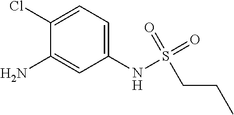

### N-(3-Amino-4-chlorophenyl)propane-1-sulfonamide

Step A:

To 4-chloro-3-nitroaniline (29.0 mL, 5.79 mmol) in DCM (30 mL) at 0° C. was added triethylamine (4.19 mL, 29.0 mmol) and propane-1-sulfonyl chloride (1.63 mL, 14.5 mmol). The solution was warmed to ambient temperature and stirred for 1 hour before dilution with aqueous bicarbonate (50 mL) and extraction with EtOAc (3×40 mL). The organics were dried over sodium sulfate, filtered, and concentrated under reduced pressure to afford N-(4-chloro-3-nitrophenyl-N-(propylsulfonyl)propane-1-sulfonamide (2.4 g, 107%), which was used without further purification.

Step B:

To N-(4-chloro-3-nitrophenyl)-N-(propylsulfonyl)propane-1-sulfonamide (2.4 g, 6.6 mmol) in 4:1 THF:MeOH (75 mL) was added 2 M NaOH (16 mL, 33 mmol). The solution was warmed to 50° C. for 3 hours. The cooled solution was concentrated under reduced pressure, and the residue was diluted with aqueous ammonium chloride (100 mL) and extracted with EtOAc (3×40 mL). The combined organic extracts were washed with aqueous bicarbonate (2×50 mL), then dried over sodium sulfate, filtered, and concentrated under reduced pressure to afford N-(4-chloro-3-nitrophenyl)propane-1-sulfonamide (1.5 g, 88%).

Step C:

To N-(4-chloro-3-nitrophenyl)propane-1-sulfonamide (0.50 g, 1.79 mmol) in MeOH (10 mL) was added 2 M HCl (2 mL) and Fe(0) (0.301 g, 5.38 mmol). The suspension was warmed to reflux for 4 hours, then cooled and filtered through GF/F paper. The filtrate was concentrated under reduced pressure to afford N-(3-amino-4-chlorophenyl)propane-1-sulfonamide (0.40 g, 89%). ¹H NMR (400 MHz, MeOD-d₄) δ 7.16-7.21 (m, 1H), 6.90 (s, 1H), 6.58-6.63 (m, 1H), 3.02-3.08 (m, 2H), 2.13 (s, 3H), 1.73-1.84 (m, 2H), 0.97-1.05 (m, 3H). LC/MS: m/z 247.1 [M−1].

### Example AE

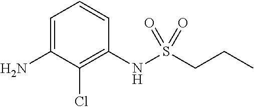

### N-(3-Amino-2-chlorophenyl)propane-1-sulfonamide

Step A:

2-Chloro-3-nitroaniline (Sienkowska, et. al., Tetrahedron 56 (2000) 165) (0.36 g, 2.086 mmol) was dissolved in DCM (20 mL) and cooled to 0° C. Triethylamine (0.8723 mL, 6.258 mmol) was added followed by propane-1-sulfonyl chloride (0.5847 mL, 5.215 mmol) and the reaction was stirred at room temperature overnight. The reaction was quenched with 0.1 N HCl (10 mL) and the layers were separated. The organic layer was dried over Na₂SO₄, and concentrated to give N-(2-chloro-3-nitrophenyl)-N-(propylsulfonyl)propane-1-sulfonamide as an oil which was used directly in the next step.

Step B:

N-(2-Chloro-3-nitrophenyl)-N-(propylsulfonyl)propane-1-sulfonamide (0.8028 g, 2.086 mmol) was dissolved in 3:1 THF/MeOH (4.0 mL) NaOH (2.0 M, 2.086 mL, 4.172 mmol) was added and the reaction was stirred for five minutes at room temperature. The reaction was quenched with 0.1N HCl (5 mL) and the volatiles were removed by rotary evaporation. EtOAc (10 mL) was added and the organic layer was washed with water and brine, dried with Na₂SO₄ and concentrated to give N-(2-chloro-3-nitrophenyl)propane-1-sulfonamide as an oil which was used directly in the next step.

Step C:

N-(2-chloro-3-nitrophenyl)propane-1-sulfonamide (0.580 g, 2.08 mmol) was dissolved in 4:1 EtOH/water (10 mL). Fe(0) (1.16 g, 20.8 mmol) was added followed by a catalytic amount of NH₄Cl (5 mg) and the reaction was heated to 80° C. for 3 hours. The reaction was cooled to room temperature, filtered through celite, concentrated, dissolved in EtOAc, washed with water, dried over Na₂SO₄ and concentrated. Purification by silica gel chromatography (10% to 90% EtOAc/Hex) gave N-(3-amino-2-chlorophenyl)propane-1-sulfonamide (259 mg, 1.04 mmol, 51%). ¹H NMR (400 MHz, DMSO-d₆) δ 9.06 (br s, 1H), 6.96-6.99 (d, 1H), 6.63-6.66 (m, 2H), 5.43 (bs, 1H), 3.03-3.07 (t, 1H), 1.71-1.77 (m, 2H), 0.94-0.98 (t, 3H); m/z (APCI-neg) M−1=247.1, 249.0.

### Example AF

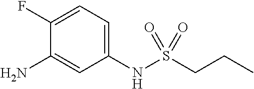

### N-(3-Amino-4-fluorophenyl)propane-1-sulfonamide

A solution of N-(3-Amino-2-chloro-4-fluorophenyl)propane-1-sulfonamide (668 mg, 2.5 mmol) dissolved in methanol (100 mL) was passed through an H-Cube hydrogenator at 50° C. and 10 bar H₂ pressure at 1 mL/minute flow rate. Solvent was removed to obtain 481 mg (83%) of N-(3-amino-4-fluorophenyl)propane-1-sulfonamide. ¹H NMR (500 MHz, DMSO-d₆) δ 9.37 (s, 1H), 6.89 (dd, J=11.2, 8.7, 1H), 6.67 (dd, J=8.1, 2.6, 1H), 6.49-6.24 (m, 1H), 5.19 (s, 2H), 3.09-2.86 (m, 2H), 1.67 (dq, J=15.0, 7.5, 2H), 0.93 (t, J=7.4, 3H). LC-MS [M+1] m/z 233.1.

### Example AG

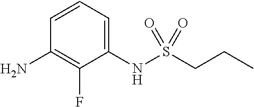

### N-(3-Amino-2-fluorophenyl)propane-1-sulfonamide

A solution of N-(3-Amino-4-chloro-2-fluorophenyl)propane-1-sulfonamide (477 mg, 1.8 mmol) dissolved in methanol (100 mL) was passed through an H-Cube hydrogenator at RT and ambient pressure at 1 mL/minute flow rate. Solvent was removed to obtain 251 mg (60%) of N-(3-amino-2-fluorophenyl)propane-1-sulfonamide. ¹H NMR (500 MHz, DMSO) δ 9.29 (s, 1H), 6.79 (t, J=8.0, 1H), 6.58 (td, J=8.1, 1.4, 1H), 6.55-6.49 (m, 1H), 5.17 (s, 2H), 3.02 (dd, J=8.7, 6.7, 2H), 1.85-1.60 (m, 2H), 0.96 (t, J=7.4, 3H). LC-MS [M+1] m/z 233.1.

### Example AH

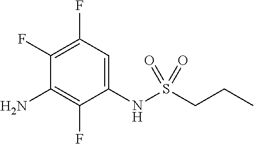 [SMILES: CCCS(=O)(=O)Nc1cc(F)c(F)c(N)c1F]

### N-(3-Amino-2,4,5-trifluorophenyl)propane-1-sulfonamide

2,4,5-Trifluorobenzene-1,3-diamine (1116 mg, 6.88 mmol) was dissolved in methylene chloride (27 mL, 420 mmol) and pyridine (557 ul, 6.88 mmol) was added. After cooling the mixture to 0° C., propane-1-sulfonyl chloride (772 ul, 6.88 mmol) was added drop-wise through a syringe. The ice bath was removed and the mixture was stirred at ambient temperature overnight. The solvent was removed under reduced pressure and the crude product purified via chromatography eluting with 1:1 ethyl acetate/hexane to afford N-(3-amino-2,4,5-trifluorophenyl)propane-1-sulfonamide (1847 mg, 83.6%). ¹H NMR (400 MHz, DMSO-d₆) δ 9.58 (s, 1H), 6.53 (dt, J=11.8, 7.5 Hz, 1H), 5.75 (s, 2H), 3.10-2.91 (m, 2H), 1.72 (dd, J=15.1, 7.5 Hz, 2H), 0.96 (t, J=7.4 Hz, 3H). LC-MS [M+1] m/z 269.0.

### Example AI

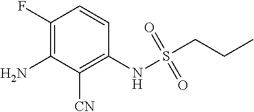 [SMILES: CCCS(=O)(=O)Nc1ccc(F)c(N)c1C#N]

### N-(3-Amino-2-cyano-4-fluorophenyl)propane-1-sulfonamide

Step A:

To 2,3,6-trifluorobenzonitrile (2.0 g, 12.7 mmol) in 5 mL isopropanol was added concentrated ammonium hydroxide (5.16 mL, 76.4 mmol). The solution was heated at 80° C. in a sealed vial overnight. The reaction mixture was concentrated and the residue partitioned between EtOAc and water. The EtOAc was washed with brine, dried over MgSO₄, filtered, and evaporated to yield 2-amino-3,6-difluorobenzonitrile (1.93 g, 12.5 mmol, 98.4% yield).

Step B:

To propane-1-sulfonamide (1.68 g, 13.6 mmol) in 10 mL DMSO with water bath cooling was added slowly in portions, 60% sodium hydride (0.558 g, 14.0 mmol). After gas evolution ceased, the mixture diluted with 5 mL DMSO to aid in dissolution, and was stirred an additional 30 minutes at ambient temperature. To the reaction was added a solution of 2-amino-3,6-difluorobenzonitrile (1.00 g, 6.49 mmol) in 20 mL DMSO, and the resulting mixture was heated at 100° C. for 20 hours then 120° C. for 16 hours. The reaction mixture was diluted with 0.5 M NaOH and washed with 2 portions EtOAc. The aqueous layer was acidified with 12M HCl to pH 4 and extracted twice with EtOAc. The organic layer was washed with 3 times with brine, dried over MgSO₄, filtered, and evaporated to yield 0.41 g. The crude product was purified by chromatography on a 50 g Biotage SNAP column with 1:1 hexane:EtOAc to afford N-(3-amino-2-cyano-4-fluorophenyl)propane-1-sulfonamide (0.33 g, 1.28 mmol, 19.8% yield). ¹H NMR (400 MHz, CDCl₃) δ 7.12-7.18 (m, 1H), 6.90-6.94 (m, 1H), 6.50 (br s, 1H), 4.58 (br s, 2H), 3.11-3.15 (m, 2H), 1.85-1.95 (m, 2H), 1.06 (t, 3H). m/z 256.1 (LC/MS negative ionization) [M−1].

### Example AJ

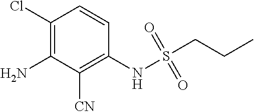 [SMILES: CCCS(=O)(=O)Nc1ccc(Cl)c(N)c1C#N]

### N-(3-Amino-4-chloro-2-cyanophenyl)propane-1-sulfonamide

Step A:

To 3-chloro-2,6-difluorobenzonitrile (2.00 g, 11.5 mmol) in 5 mL isopropanol was added 14.8M ammonium hydroxide (4.67 mL, 69.1 mmol). The colorless solution was heated at 80° C. in a sealed vial. After 2 hours the reaction mixture was concentrated and the residue partitioned between EtOAc and water. The EtOAc was washed with brine, dried over MgSO₄, filtered, and evaporated to yield 2-amino-3-chloro-6-fluorobenzonitrile (1.63 g, 9.56 mmol, 82.9% yield).

Step B:

To propane-1-sulfonamide (0.740 g, 6.01 mmol) in 10 mL NMP with water bath cooling was added slowly in portions, 60% sodium hydride (0.252 g, 6.30 mmol). The mixture was stirred an additional 30 minutes at ambient temperature, then heated 1 hour at 40° C. The mixture was cooled to room temperature and 2-amino-3-chloro-6-fluorobenzonitrile (0.50 g, 2.93 mmol) was added. The resulting mixture was heated in a sealed vial at 120° C. overnight. The reaction mixture was diluted with 0.5 M NaOH and washed twice with EtOAc. The aqueous layer was acidified with 12 M HCl to pH 5 and extracted with 2 portions of EtOAc. The combined EtOAc extracts were washed twice with brine, dried over MgSO₄, filtered, and evaporated to afford 0.65 g of crude product, which was chromatographed on a 50 g Biotage SNAP column with DCM to yield N-(3-amino-4-chloro-2-cyanophenyl)propane-1-sulfonamide (0.29 g, 1.06 mmol, 36.1% yield). ¹H NMR (400 MHz, CDCl₃) δ 7.40 (d, 1H), 6.97 (d, 1H), 6.62 (br s, 1H), 4.90 (br s, 2H), 3.13-3.17 (m, 2H), 1.85-1.94 (m, 2H), 1.06 (t, 3H). m/z 272.1 (LC/MS negative ionization) [M−1].

### Example AK

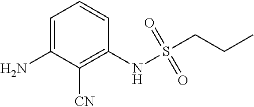 [SMILES: CCCS(=O)(=O)Nc1cccc(N)c1C#N]

### N-(3-Amino-2-cyanophenyl)propane-1-sulfonamide

To propane-1-sulfonamide (0.950 g, 7.71 mmol) in 7 mL N-methylpyrrolidone (“NMP”) in a vial was added 60% sodium hydride (0.194 g, 8.08 mmol). After gas evolution ceased, the mixture was stirred 30 minutes at 40° C., then 2-amino-6-fluorobenzonitrile (0.500 g, 3.67 mmol) was added and the sealed vial was heated at 120° C. overnight, then 150° C. overnight, then for 3 days at 150° C. The reaction mixture was partitioned between 0.5 M NaOH and EtOAc. The aqueous layer was acidified to pH 5 with concentrated HCl and extracted with EtOAc. The EtOAc extract was washed with twice with brine, dried over MgSO₄, filtered, and evaporated to yield 0.73 g. The material was dissolved in ether and washed with 3 portions water to remove NMP, dried over MgSO₄, filtered, and evaporated to yield N-(3-amino-2-cyanophenyl)propane-1-sulfonamide (0.34 g, 1.42 mmol, 38.7% yield). ¹H NMR (400 MHz, CDCl₃) δ 7.29 (t, 1H), 6.98 (dd, 1H), 6.70 (br s, 1H), 6.51 (dd, 1H), 4.52 (br s, 2H), 3.18-3.14 (m, 2H), 1.95-1.85 (m, 2H), 1.06 (t, 3H). m/z 238.1 (LC/MS negative ionization) [M−1].

### Example AL

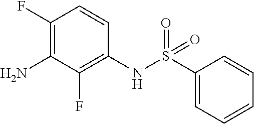 [SMILES: Nc1c(F)ccc(NS(=O)(=O)c2ccccc2)c1F]

### N-(3-Amino-2,4-difluorophenyl)benzenesulfonamide

Step A:

Methyl 3-amino-2,6-difluorobenzoate (1.14 g, 6.092 mmol) was dissolved in DCM (30.5 mL) and treated sequentially with triethylamine (2.50 mL, 18.27 mmol) and benzenesulfonyl chloride (1.63 mL, 12.79 mmol). The reaction mixture was stirred at ambient temperature for 4 hours and then diluted with additional DCM and washed with water (2×) and brine (1×). The organic phase was dried over Na₂SO₄ and concentrated to provide methyl 2,6-difluoro-3-(N-(phenylsulfonyl)phenylsulfonamido)benzoate (2.848 g, 6.092 mmol). The crude material was then immediately dissolved in 60.9 mL 4:1 THF:MeOH (0.1 M) and treated with 2.0 M KOH (15.23 mL, 30.46 mmol). The reaction mixture was stirred at ambient temperature for 2 hours. The organic solvent was removed under reduced pressure and the aqueous residue acidified to pH 3 using 1.0 M HCl. Extraction with EtOAc (2×) was followed by washing the combined organic extracts with water (2×). The crude product was then extracted as its carboxylate salt with 1.0 M NaOH (2×). The combined aqueous NaOH extracts were acidified to pH 3 using 6.0 M HCl and extracted with EtOAc (2×). The combined organic extracts were washed with water (2×) and brine (1×) and then dried over Na₂SO₄ and concentrated to afford 2,6-difluoro-3-(phenylsulfonamido)benzoic acid (1.53 g, 4.884 mmol, 80.17% yield). LC/MS: m/z 312.0 [M−1].

Step B:

2,6-Difluoro-3-(phenylsulfonamido)benzoic acid (1.53 g, 4.884 mmol) was dissolved in 25 mL DMF (25 mL) and treated sequentially with triethylamine (1.99 mL, 14.65 mmol) and then diphenylphosphoryl azide (1.633 mL, 7.326 mmol). The reaction mixture was stirred at ambient temperature for 1 hour and then treated with 10 mL water and heated to 80° C. for 16 hours. The reaction mixture was cooled to ambient temperature and diluted with water. Extraction with EtOAc (2×) and washing of the combined organic phases with water (4×) and brine (1×) was followed by drying over Na₂SO₄ and concentration under reduced pressure. Purification by flash chromatography eluting with a gradient of 10-70% EtOAc in hexanes afforded N-(3-amino-2,4-difluorophenyl)benzenesulfonamide (508.9 mg, 1.790 mmol, 35.65% yield). LC/MS: m/z 283.1 [M−1].

### Example AM

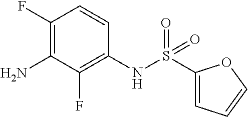 [SMILES: Nc1c(F)ccc(NS(=O)(=O)c2ccco2)c1F]

### N-(3-Amino-2,4-difluorophenyl)furan-2-sulfonamide

Step A:

Methyl 3-amino-2,6-difluorobenzoate (652.8 mg, 3.488 mmol) was dissolved in 17.4 mL DCM (0.2 M) and treated sequentially with triethylamine (1.42 mL, 10.46 mmol) and furan-2-sulfonyl chloride (1.162 g, 6.976 mmol). The reaction mixture was stirred at ambient temperature for 16 hours and then diluted with additional DCM and washed with water (2×) and brine (1×). The organic phase was dried over Na₂SO₄ and concentrated to provide methyl 2,6-difluoro-3-(N-(furan-2-ylsulfonyl)furan-2-sulfonamido)benzoate (1.561 g, 3.489 mmol). The crude material was then immediately dissolved in 17.5 mL 4:1 THF:MeOH (0.2 M) and treated with 2.0 M KOH (8.7 mL, 17.45 mmol). The reaction mixture was stirred at ambient temperature for 2 hours. The organic solvent was removed under reduced pressure and the aqueous residue acidified to pH 3 using 1.0 M HCl. Extraction with EtOAc (2×) was followed by washing the combined organic extracts with water (2×). The crude product was then extracted as its carboxylate salt with 1.0 M NaOH (2×). The combined aqueous NaOH extracts were acidified to pH 3 using 6.0 M HCl and extracted with EtOAc (2×). The combined organic extracts were washed with water (2×) and brine (1×) and then dried over Na₂SO₄ and concentrated to afford 2,6-difluoro-3-(furan-2-sulfonamido)benzoic acid (475.0 mg, 1.566 mmol, 44.91% yield). LC/MS: m/z 302.0 [M−1].

Step B:

2,6-difluoro-3-(furan-2-sulfonamido)benzoic acid (475.0 mg, 1.566 mmol) was dissolved in DMF (15.7 mL) and treated sequentially with triethylamine (0.637 mL, 4.699 mmol) and then diphenylphosphoryl azide (0.524 mL, 2.350 mmol). The reaction mixture was stirred at ambient temperature for 1 hour and then treated with 5 mL water and heated to 80° C. for 16 hours. The reaction mixture was cooled to ambient temperature and diluted with water. Extraction with EtOAc (2×) and washing of the combined organic phases with water (4×) and brine (1×) was followed by drying over Na₂SO₄ and concentration under reduced pressure. Purification via flash chromatography eluting with a gradient of 5->60% EtOAc:hexanes afforded N-(3-amino-2,4-difluorophenyl)furan-2-sulfonamide (152.6 mg, 0.556 mmol, 35.52% yield). LC/MS: m/z 273.1 [M−1].

### Example AN

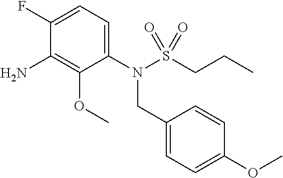 [SMILES: CCCS(=O)(=O)N(Cc1ccc(OC)cc1)c1ccc(F)c(N)c1OC]

### N-(3-Amino-4-fluoro-2-methoxyphenyl)-N-(4-methoxybenzyl)propane-1-sulfonamide

Step A:

A 250 mL round bottom flask was charged with methyl 2,6-difluoro-3-nitrobenzoate (10.03 g, 46.18 mmol) and methanol (60 mL, 1000 mmol) and was then cooled over a brine/ice bath at −4° C. for 20 minutes. A 5 M solution of sodium methoxide in methanol (11.98 mL, 59.88 mmol) was added to this solution drop wise over 20 minutes while maintaining the reaction temperature at −4° C. over the course of the addition. The reaction mixture was allowed to stir overnight, gradually rising to room temperature. The methanol was removed under reduced pressure and the residual oil quenched with a saturated aqueous solution of potassium bicarbonate (250 mL). The organic layer was saved and the aqueous layer extracted twice with ethyl acetate (250 mL). The combined organic layers were washed once with brine, dried over magnesium sulfate, filtered, and concentrated. The crude product was purified via flash chromatography (330 g ISCO column) using a gradient of 0-50% ethyl acetate: heptane to yield methyl 6-fluoro-2-methoxy-3-nitrobenzoate as an oil (3.37 g. 32%). ¹H NMR (400 MHz, DMSO-d₆) δ=8.23 (dd, J=9.3, 5.9, 1H), 7.39 (t, J=8.9, 1H), 3.94 (s, 3H), 3.89 (s, 3H).

Step B:

A 250 mL round bottom flask was charged with methyl 6-fluoro-2-methoxy-3-nitrobenzoate (3.37 g, 14.71 mmol) dissolved in methanol (125 mL, 3080 mmol). Nitrogen was passed through the reaction mixture, and 10% palladium on activated carbon (1.3 g, 1.2 mmol) was added. The flask was capped and evacuated and then allowed to stir for 60 hours under an atmosphere of hydrogen at ambient temperature and pressure. The mixture was then filtered through Celite® to remove the solid catalyst and washed with methanol (500 mL). The filtrate was concentrated to give methyl 3-amino-6-fluoro-2-methoxybenzoate as an oil (2.95 g, 100%). ¹H NMR (400 MHz, DMSO-d₆) δ=6.74-6.84 (m, 2H), 4.98 (s, 2H), 3.85 (s, 3H), 3.68 (s, 3H).

Step C:

A 250 mL round bottom flask was charged with a solution of methyl 3-amino-6-fluoro-2-methoxybenzoate (3.656 g, 18.36 mmol) in methylene chloride (100 mL). To this reaction mixture was added a solution of 4-dimethylaminopyridine (113 mg, 0.925 mmol), pyridine (7.45 mL, 92.1 mmol) and propane-1-sulfonyl chloride (8.25 mL, 73.6 mmol) in methylene chloride (10 mL) over a course of five minutes. The reaction mixture was stirred at room temperature for 14 hours. After removing the organic solvent under reduced pressure, 100 mL saturated aqueous sodium bicarbonate was added followed by stirring for 10 minutes. The aqueous mixture was extracted with 200 mL ethyl acetate (2×). The combined organic layers were washed with brine, dried over magnesium sulfate, filtered and concentrated. The crude product was purified using flash chromatography, eluting with 0-30% ethyl acetate/heptanes to give methyl 6-fluoro-2-methoxy-3-(propylsulfonamido)benzoate as an oil (4.914 g, 85%). ¹H NMR (400 MHz, DMSO-d₆) δ=9.27 (s, 1H), 7.48 (dd, J=9.1, 6.1, 1H), 7.09 (t, J=9.0, 1H), 3.89 (s, 3H), 3.85-3.74 (m, 3H), 3.29 (s, 15H).

Step D:

A 100 mL round bottom flask was charged with methyl 6-fluoro-2-methoxy-3-(propylsulfonamido)benzoate (4.91 g, 16.1 mmol) dissolved in N,N-dimethylformamide (16 mL, 210 mmol) and was cooled over an ice/brine bath. Sodium hydride (0.676 g, 16.9 mmol) was added in four portions. After the vigorous bubbling subsided, the reaction mixture was stirred for 1 h at room temperature. The reaction mixture was cooled over an ice/brine bath, and p-methoxybenzyl chloride (2.646 g, 16.90 mmol) was added. The reaction was allowed to warm to room temperature over the next three hours and then as quenched by adding a semi-saturated aqueous ammonium chloride solution (200 mL) at 0° C. After stirring at room temperature overnight, the aqueous layer was discarded and the remaining oil washed with heptanes to remove the mineral oil. The residual oil was dissolved in ethyl acetate, dried over magnesium sulfate, filtered and concentrated to remove the ethyl acetate. The crude product was purified by flash chromatography (120 g column), using a gradient of 0-100% ethyl acetate: heptanes to give methyl 6-fluoro-2-methoxy-3-(N-(4-methoxybenzyl)propylsulfonamido)benzoate as an oil (3.71 g, 57%). ¹H NMR (400 MHz, DMSO-d₆) δ=7.28 (dd, J=9.0, 6.3 Hz, 1H), 7.12 (d, J=8.7 Hz, 2H), 6.98 (t, J=8.9 Hz, 1H), 6.84 (dd, J=6.8, 4.8 Hz, 2H), 4.65 (s, 2H), 3.90 (d, J=7.6 Hz, 3H), 3.73 (s, 3H), 3.70 (s, 3H), 3.28-3.21 (m, 2H), 1.84-1.70 (m, 2H), 1.00 (q, J=7.2 Hz, 3H).

Step E:

A 250 mL round bottom flask was charged with methyl 6-fluoro-2-methoxy-3-(N-(4-methoxybenzyl)propylsulfonamido)benzoate (4.42 g, 10.4 mmol) dissolved in tetrahydrofuran (70 mL, 900 mmol). 1M of sodium hydroxide in water (67.8 mL, 67.8 mmol) was added, and the mixture was stirred at 60° C. for 48 hours. After cooling, the THF was removed under reduced pressure. The basic aqueous solution was diluted with water to a volume of 100 mL and then extracted once with ethyl acetate (200 mL). The aqueous layer was acidified with concentrated hydrochloric acid (5 mL) to a pH of 2 and extracted three times with of ethyl acetate (100 mL). The combined organic layers were dried over magnesium sulfate, filtered and concentrated to afford 6-fluoro-2-methoxy-3-(N-(4-methoxybenzyl)propylsulfonamido)benzoic acid as a solid (4.2529 g, 99%). ¹H NMR (400 MHz, DMSO-d₆) δ=13.86 (s, 1H), 7.19 (dd, J=8.9, 6.3 Hz, 1H), 7.12 (d, J=8.6 Hz, 2H), 6.93 (t, J=8.8 Hz, 1H), 6.83 (d, J=8.7 Hz, 2H), 4.65 (s, 2H), 3.80 (s, 3H), 3.70 (s, 3H), 3.27-3.19 (m, 2H), 1.78 (dd, J=15.3, 7.5 Hz, 2H), 1.01 (t, J=7.4 Hz, 3H).

Step F:

Under a nitrogen atmosphere, a dry 100 mL round bottom, stir bar and reflux condenser were charged with 6-fluoro-2-methoxy-3-(N-(4-methoxybenzyl)propysulfonamido)benzoic acid (379 mg, 0.921 mmol) dissolved in 1,4-dioxane (10 mL, 83 mmol). Triethylamine (295.3 uL, 2.12 mmol) then diphenylphosphonic azide (228.3 uL, 1.06 mmol) were added. The reaction mixture was stirred at room temperature for 3 hours and then heated to reflux for 1 hour. Water (10 mL, 36 mmol) was added to reaction mixture and heating to reflux continued for 2 hours. The reaction mixture was concentrated to remove the 1,4 dioxane. Residual material was stirred with a saturated aqueous solution of sodium bicarbonate for thirty minutes and the aqueous layer was decanted and discarded. The residual oil was purified by flash chromatography (40 g column) using a gradient of 0-100% ethyl acetate:heptanes to give N-(3-amino-4-fluoro-2-methoxyphenyl)-N-(4-methoxybenzyl)propane-1-sulfonamide as an oil (108 mg, 31%). ¹H NMR (400 MHz, DMSO-d₆) δ=7.11 (d, J=8.7 Hz, 2H), 6.82 (d, J=8.7 Hz, 2H), 6.73 (dd, J=10.4, 8.9 Hz, 1H), 6.33 (dd, J=8.9, 5.8 Hz, 1H), 4.95 (s, 2H), 4.63 (s, 2H), 3.69 (s, 3H), 3.62 (s, 3H), 3.25-3.17 (m, 2H), 1.77 (dq, J=15.0, 7.4 Hz, 2H), 1.00 (t, J=7.4 Hz, 3H). MS m/z 383.2 [M+1].

### Example AO

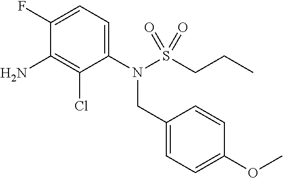 [SMILES: CCCS(=O)(=O)N(Cc1ccc(OC)cc1)c1ccc(F)c(N)c1Cl]

### N-(3-Amino-2-chloro-4-fluorophenyl)-N-(4-methoxybenzyl)propane-1-sulfonamide

N-(3-Amino-2-chloro-4-fluorophenyl)propane-1-sulfonamide (75 g, 280 mmol) was dissolved in N,N-dimethylformamide (200 mL, 2000 mmol). A 60% sodium hydride suspension in mineral oil (6:4, sodium hydride:mineral oil, 11.85 g, 296 mmol) was added in multiple portions over a period of fifteen minutes. The reaction mixture was stirred at room temperature for 90 minutes and was then warmed to 40° C. for 2 hours. This homogeneous mixture was cooled to 0° C. and p-methoxybenzyl chloride (40.03 mL, 295.25 mmol) was added over 5 minutes. The reaction was left to stir and warm to room temperature. After 14 hours, the reaction mixture was poured into a dilute ammonium chloride solution (1750 mL) and the water layer was decanted to leave an oil. This oil was triturated three times with water (2 L). The remaining product was transferred into a 1 L beaker, diluted with 800 mL water, sonicated for 30 minutes and then stirred at room temperature for 1 hour. The resulting solid was collected via filtration and dried by lyophilization to give 111.9 g (99%) of N-(3-amino-2-chloro-4-fluorophenyl)-N-(4-methoxybenzyl)propane-1-sulfonamide. ¹H NMR (500 MHz, DMSO-d₆) δ 7.11 (d, J=8.6 Hz, 2H), 6.96 (dd, J=10.6, 8.8 Hz, 1H), 6.81 (t, J=5.7 Hz, 2H), 6.51 (dd, J=8.7, 5.1 Hz, 1H), 5.42 (s, 2H), 4.71 (d, J=14.4 Hz, 1H), 4.57 (d, J=14.4 Hz, 1H), 3.70 (s, 3H), 3.21 (td, J=6.7, 1.4 Hz, 2H), 1.77 (dd, J=15.3, 7.5 Hz, 2H), 1.00 (t, J=7.4 Hz, 3H). MS m/z 387.2 [M+1].

### Example AP

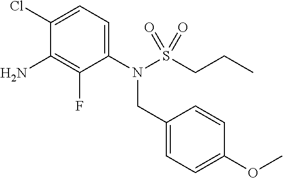 [SMILES: CCCS(=O)(=O)N(Cc1ccc(OC)cc1)c1ccc(Cl)c(N)c1F]

### N-(3-Amino-4-chloro-2-fluorophenyl)-N-(4-methoxybenzyl)propane-1-sulfonamide

N-(3-Amino-4-chloro-2-fluorophenyl)-N-(4-methoxybenzyl)propane-1-sulfonamide was prepared according to the general procedure for Example AN, substituting N-(3-amino-4-chloro-2-fluorophenyl)propane-1-sulfonamide for N-(3-amino-2-chloro-4-fluorophenyl)-propane-1-sulfonamide. ¹H NMR (400 MHz, CDCl₃) δ 7.15 (d, J=8.6 Hz, 2H), 6.92 (dd, J=8.7, 1.8 Hz, 1H), 6.78 (t, J=8.6 Hz, 2H), 6.44 (t, J=8.3 Hz, 1H), 4.70 (s, 2H), 4.02 (broad s, 2H), 3.77 (s, 3H), 3.08-3.02 (m, 2H), 2.02-1.85 (m, 2H), 1.06 (t, J=7.4 Hz, 3H). MS m/z 387.1 [M+1].

### Example AQ

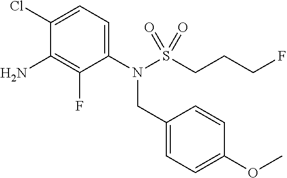 [SMILES: COc1ccc(CN(c2ccc(Cl)c(N)c2F)S(=O)(=O)CCCF)cc1]

### N-(3-Amino-4-chloro-2-fluorophenyl)-3-fluoro-N-(4-methoxybenzyl)propane-1-sulfonamide

N-(3-Amino-4-chloro-2-fluorophenyl)-3-fluoro-N-(4-methoxybenzyl)propane-1-sulfonamide was prepared according to the general procedure for Example AN, substituting N-(3-amino-4-chloro-2-fluorophenyl)-3-fluoropropane-1-sulfonamide for N-(3-amino-2-chloro-4-fluorophenyl)propane-1-sulfonamide. ¹H NMR (500 MHz, CDCl₃) δ 7.15 (d, J=8.4 Hz, 2H), 6.93 (d, J=8.9 Hz, 1H), 6.79 (d, J=8.5 Hz, 2H), 6.44 (t, J=8.2 Hz, 1H), 4.71 (s, 2H), 4.64-4.58 (m, 1H), 4.52 (t, J=5.7 Hz, 1H), 4.11 (broad s, 2H), 3.78 (s, 3H), 3.25-3.19 (m, 2H), 2.35-2.21 (m, 2H). MS m/z 404.8 [M+1].

### Example AR

### N-(3-Amino-2,4-fluorophenyl)-N-(4-methoxybenzyl)propane-1-sulfonamide

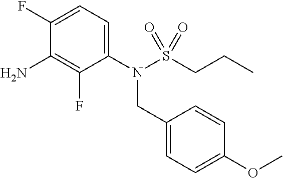 [SMILES: CCCS(=O)(=O)N(Cc1ccc(OC)cc1)c1ccc(F)c(N)c1F]

### N-(3-Amino-2,4-fluorophenyl)-N-(4-methoxybenzyl)propane-1-sulfonamide

N-(3-Amino-2,4-difluorophenyl)-N-(4-methoxybenzyl)propane-1-sulfonamide was prepared according to the general procedure for Example AN, substituting N-(3-amino-2,4-difluorophenyl)propane-1-sulfonamide for N-(3-amino-2-chloro-4-fluorophenyl)propane-1-sulfonamide. ¹H NMR (400 MHz, DMSO-d₆) δ 7.13 (m, 2H), 6.92-6.76 (m, 3H), 6.49 (td, J=8.5, 5.6 Hz, 1H), 5.25 (s, 2H), 4.64 (s, 2H), 3.70 (s, 3H), 3.25-3.16 (m, 2H), 1.85-1.69 (m, 2H), 1.00 (t, J=7.4 Hz, 3H).

### Example AS

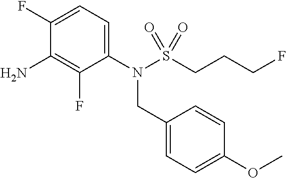 [SMILES: COc1ccc(CN(c2ccc(F)c(N)c2F)S(=O)(=O)CCCF)cc1]

### N-3-Amino-2,4-difluorophenyl)-3-fluoro-N-(4-methoxybenzyl)propane-1-sulfonamide

N-(3-Amino-2,4-difluorophenyl)-3-fluoro-N-(4-methoxybenzyl)propane-1-sulfonamide was prepared according to the general procedure for Example AN, substituting N-(3-amino-2,4-difluorophenyl)-3-fluoropropane-1-sulfonamide for N-(3-amino-2-chloro-4-fluorophenyl)propane-1-sulfonamide. ¹H NMR (400 MHz, DMSO-d₆) δ 7.13 (d, J=8.6 Hz, 2H), 6.89-6.75 (m, 3H), 6.51 (td, J=8.5, 5.6 Hz, 1H), 5.27 (s, 2H), 4.66 (s, 2H), 4.62 (t, J=5.9 Hz, 1H), 4.50 (t, J=5.9 Hz, 1H), 3.70 (s, 3H), 3.34 (dd, J=8.9, 6.6 Hz, 2H), 2.22-2.06 (m, 2H).

### Example AT

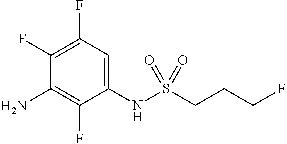 [SMILES: Nc1c(F)c(F)cc(NS(=O)(=O)CCCF)c1F]

### N-(3-Amino-2,4,5-trifluorophenyl)-3-fluoropropane-1-sulfonamide

To a stirred solution of 2,4,5-trifluorobenzene-1,3-diamine (1116 mg, 6.88 mmol) in methylene chloride (27 ml, 420 mmol) was added pyridine (557 ul, 6.88 mmol). The reaction mixture was cooled to 0° C. and 3-fluoropropane-1-sulfonyl chloride (762 ul, 6.88 mmol) was added drop-wise. The ice bath was removed and the mixture was stirred at RT overnight. The organics were removed via reduced pressure and the crude product was purified through column chromatography eluted with 1:1 ethyl acetate/hexane to give N-(3-amino-2,4,5-trifluorophenyl)-3-fluoropropane-1-sulfonamide (628 mg, 32%). ¹H NMR (400 MHz, DMSO) δ 9.72 (s, 1H), 6.54 (dt, J=12.1, 7.4 Hz, 1H), 5.78 (s, 2H), 4.60 (t, J=5.9 Hz, 1H), 4.48 (t, J=5.9 Hz, 1H), 3.26-3.13 (m, 2H), 2.19-1.99 (m, 2H); LC-MS [M+1] m/z 287.0.

### Example AU

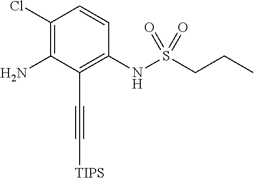

### N-(3-Amino-4-chloro-2-((triisopropylsilyl)ethynyl)phenyl)propane-1-sulfonamide

Step A:

2-Chloro-1,3-dinitrobenzene (0.500 g, 2.47 mmol), CuI (0.0940 g, 0.494 mmol), P(t-Bu)₃ (1.51 mL, 0.494 mmol) and ethynyltriisopropylsilane (0.658 mL, 2.96 mmol) were dissolved in acetonitrile/TEA (10 mL; 5:1). Nitrogen gas was passed through the mixture for 5 minutes and PdCl₂(MeCN)₂ (0.0640 g, 0.247 mmol) was added; nitrogen gas passage was then continued for 10 minutes. The reaction mixture was stirred at room temperature for 2 hours, diluted with EtOAc and filtered through Celite®. The mixture was concentrated, dissolved in EtOAc, and washed with 0.1N HCl, water and brine. Following drying over Na₂SO₄ and removal of the solvent under reduced pressure, the product was purified by Biotage chromatography eluting with hexanes/EtOAc to give ((2,6-dinitrophenyl)ethynyl)triisopropylsilane (310 mg, 36%) as an oil. m/z (APCI-neg) M−1=348.1.

Step B:

((2,6-Dinitrophenyl)ethynyl)triisopropylsilane (0.310 g, 0.890 mmol) was dissolved in DCM/DMF (30 mL; 1:1). SnCl₂ dihydrate (10.0 g, 44.5 mmol) was added and the reaction mixture was stirred for 1 hour at room temperature. The mixture was poured into saturated aqueous NaHCO₃ (200 mL) giving a precipitate, which was stirred at room temperature for several minutes and filtered through Celite®. The layers were separated. The aqueous layer was extracted with DCM, and the combined organic layers were washed with water (×2) and brine, and dried over Na₂SO₄ and concentrated. The product was purified by Biotage chromatography eluting with hexanes/EtOAc to give 2-((triisopropylsilyl)ethynyl)benzene-1,3-diamine as an oil. ¹H NMR (400 MHz, CDCl₃) δ 6.89 (t, 1H), 6.09 (d, 2H), 4.17 (br s, 4H), 1.14 (s, 21H). m/z (APCI-pos) M+1=389.2.

Step C:

2-((Triisopropylsilyl)ethynyl)benzene-1,3-diamine (0.072 g, 0.249 mmol) was dissolved in THF (5 mL) and N-chlorosuccinimide (0.036 g, 0.286 mmol) was added, followed by stirring at room temperature for 1 hour. The crude reaction mixture was diluted with EtOAc, washed with water (3×) and brine, and then dried over Na₂SO₄ and concentrated. The product was purified by Biotage chromatography eluting with hexanes/DCM to give 4-chloro-2-((triisopropylsilyl)ethynyl)benzene-1,3-diamine (55 mg, 64% for two steps) as an oil. m/z (APCI-pos) M+1=323.1, 325.2.

Step D:

4-Chloro-2-((triisopropylsilyl)ethynyl)benzene-1,3-diamine (0.0554 g, 0.172 mmol) was dissolved in 10:1 dichloroethane/pyridine (1 mL) and cooled to 0° C. Propane-1-sulfonyl chloride (0.0193 mL, 0.172 mmol) was added, and the reaction was stirred at 50° C. overnight. The reaction was concentrated, dissolved in EtOAc and washed with 0.1N HCl, water, and brine, dried over Na₂SO₄ and concentrated. The product was purified by Biotage chromatography eluting with hexanes/DCM to give N-(3-amino-4-chloro-2-((triisopropylsilyl)ethynyl)phenyl)propane-1-sulfonamide (30 mg, 41%) as an oil. ¹H NMR (400 MHz, CDCl₃) δ 7.17-7.19 (d, 1H), 6.93-6.95 (d, 1H), 6.89 (br s, 1H), 4.69 (br s, 2H), 3.04-3.08 (m, 2H), 1.77-1.87 (m, 2H), 1.14-1.16 (m, 21H), 0.98-1.02 (t, 3H). m/z (APCI-neg) M−1=427.2, 429.2.

### Example AV

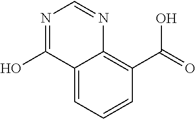 [SMILES: O=C(O)c1cccc2c(O)ncnc12]

### 4-Hydroxyquinazoline-8-carboxylic acid

A 10 L reactor was charged with 2-aminoisophthalic acid (600 g, 3.3 mol) and formamidine acetate (1035 g, 9.9 mol, 3.0 eq.). After stirring for 25 minutes, formamide (132 mL, 3.3 mol) was added. The mixture was heated at 170° C. with a sand bath and continuously stirred with a heavy duty overhead mechanical stirrer for 5 hours. HPLC analysis indicated no presence of 2-aminoisophthalic acid. The temperature was lowered to 80° C. Water (5 L) was slowly added to the reactor. The resulting suspension was heated under reflux for 1 hour. The reaction mixture was then cooled to room temperature and filtered. The filter cake was washed twice with water (2 L) and twice with MeOH (2 L). The filter cake was dried in an oven over 40° C. for 17 hours. The first crop of the final product was obtained (384 g). To the previously obtained filtrate, concentrated HCl was added and the pH adjusted to 0.2. The mixture was filtered, and the filter cake washed with water (500 mL). Drying in the oven at 40° C. for 16 hours yielded a second crop of the product which was combined with the first crop. Both product crops were combined to afford 500 g (87%) 4-hydroxyquinazoline-8-carboxylic acid. ¹H NMR (400 MHz, DMSO) δ 8.51 (s, 1H), 8.45 (dd, J=7.6, 1.6 Hz, 1H), 8.35 (dd, J=7.9, 1.6 Hz, 1H), 7.68 (t, J=7.8 Hz, 1H).

### Example AW

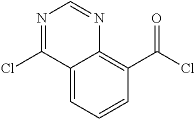 [SMILES: O=C(Cl)c1cccc2c(Cl)ncnc12]

### 4-Chloroquinazoline-8-carbonyl chloride

4-Hydroxyquinazoline-8-carboxylic acid (2.50 g, 13.1 mmol) was suspended in thionyl chloride (40 mL) and DMF (0.20 mL, 2.63 mmol) was added. The reaction mixture was heated at reflux for 2 hours, and the remaining undissolved solid was then filtered off. The filtrate was concentrated in vacuo and the residue redissolved in chloroform and re-concentrated in vacuo. The same process was repeated twice with toluene. The obtained solid was triturated with heptane, and filtered to afford 4-chloroquinazoline-8-carbonyl chloride (2.30 g, 77%). m/z (ES-MS) (M−2Cl+2−OMe)+1=219.2. ¹H NMR (400 MHz, DMSO) δ 8.56 (s, 1H), 8.48 (dd, J=7.6, 1.6 Hz, 1H), 8.39 (dd, J=7.9, 1.6 Hz, 1H), 7.71 (t, J=7.8 Hz, 1H).

### Example AX

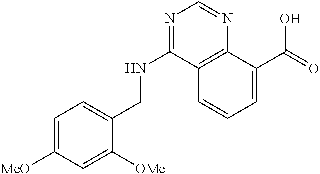 [SMILES: COc1ccc(CNc2ncnc3c(C(=O)O)cccc23)c(OC)c1]

### 4-(2,4-Dimethoxybenzylamino)quinazoline-8-carboxylic acid

Step A:

4-Hydroxyquinazoline-8-carboxylic acid (20.0 g, 105 mmol) was taken up in ethanol (1.5 L) as a slurry. Concentrated H₂SO₄ (40 mL) was added and the solution was heated to reflux giving a homogeneous solution. The reaction was heated to reflux for 3 days, cooled to room temperature and the volatiles were removed by rotary evaporation giving an oil. Water (800 mL) was added and the solution was neutralized with saturated aqueous NaHCO₃ giving a precipitate which was collected by filtration, washed with water (100 mL, 3×) and dried under high vacuum giving ethyl 4-hydroxyquinazoline-8-carboxylate (20.5 g, 93.9 mmol, 89%). ¹H NMR (400 MHz, DMSO-d₆) δ 12.43 (br s, 1H), 8.24-8.27 (dd, 1H), 8.16 (br s, 1H), 7.97 (br s, 1H), 7.55-7.59 (t, 1H), 4.32-4.37 (q, 2H), 1.30-1.34 (t, 1H).

Step B:

Ethyl 4-hydroxyquinazoline-8-carboxylate (10.0 g, 45.8 mmol) and (1H-benzo[d][1,2,3]triazol-1-yloxy)tris(dimethylamino)phosphonium hexafluorophosphate(V) (17.7 g, 59.6 mmol) and DBU (10.3 mL, 68.7 mmol) were dissolved in DMF (200 mL). The solution was stirred for 10 minutes and (2,4-dimethoxyphenyl)methanamine (10.4 mL, 68.7 mmol) was added and the reaction was stirred at room temperature overnight. The reaction was partitioned between EtOAc and water and the water layer extracted once with EtOAc. The combined organics were washed with water (4×), brine (1×), dried over Na₂SO₄ and concentrated to give ethyl 4-(2,4-dimethoxybenzylamino)quinazoline-8-carboxylate as an oil which was used directly in the next step.

Step C:

Ethyl 4-(2,4-dimethoxybenzylamino)quinazoline-8-carboxylate (16.83 g, 45.8 mmol) was dissolved in 4:1 THF/MeOH (500 mL) NaOH (2.0 M, 68.70 mL, 137.4 mmol) was added and stirred overnight at room temperature. The volatiles were removed by rotary evaporation and the aqueous solution was acidified to pH 3 using 1.0 M HCl. The aqueous solution was extracted with 20% isopropanol/DCM (2×) and the combined organics were washed with water (1×), brine (1×), dried over Na₂SO₄ and concentrated to give 4-(2,4-dimethoxybenzylamino)quinazoline-8-carboxylic acid (11.8 g, 34.8 mmol, 76%) as a tan solid. ¹H NMR (400 MHz, DMSO-d₆) δ 9.55 (br s, 1H), 8.75-8.78 (d, 1H), 8.65 (s, 1H), 8.52-8.53 (d, 1H), 7.69-7.73 (t, 1H), 7.11-7.13 (d, 1H), 6.59 (s, 1H), 6.44-6.47 (d, 1H), 4.73 (bs, 1H), 3.83 (s, 3H), 3.74 (s, 3H); m/z (APCI-neg) M−1=338.1

### Example AY

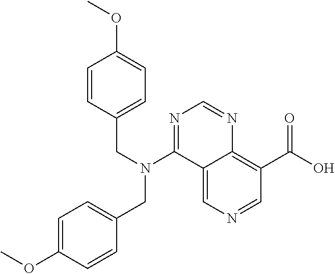 [SMILES: COc1ccc(CN(Cc2ccc(OC)cc2)c2ncnc3c(C(=O)O)cncc23)cc1]

### 4-(Bis(4-methoxybenzyl)amino)pyrido[4,3-d]pyrimidine-8-carboxylic acid

Step A:

A suspension of 4-amino-5-bromonicotinic acid (U.S. Pat. No. 3,950,160) (5.00 g, 23.0 mmol) in formamide (6.4 mL) was heated at 180° C. for 4 hours and then cooled to room temperature. Water was added, and the solid was filtered and dried under high vacuum to afford 8-bromopyrido[4,3-d]pyrimidin-4-ol (1.95 g, 37%).

Step B:

8-Bromopyrido[4,3-d]pyrimidin-4-ol (0.82 g, 3.62 mmol) was suspended in thionyl chloride (36 mL) and DMF was added (0.28 mL, 3.62 mmol). The reaction mixture was stirred at reflux for 5 hours and then concentrated under reduced pressure. Toluene was added, and the mixture was re-concentrated in vacuo (repeated twice) to afford crude 8-bromo-4-chloropyrido[4,3-d]pyrimidine (0.89 g, quantitative yield) which was used in the next step without further purification.

Step C:

A microwave vessel was charged with 8-bromo-4-chloropyrido[4,3-d]pyrimidine (0.43 g, 1.74 mmol), bis(4-methoxybenzyl)amine (WO 2007/028129) (1.07 g, 4.17 mmol) and THF (4.2 mL). The reaction mixture was heated in a microwave reactor at 90° C. for 15 minutes. The reaction was concentrated in vacuo, and the crude product purified by flash chromatography using 10% MeOH/EtOAc to afford 8-bromo-N,N-bis(4-methoxybenzyl)pyrido[4,3-d]pyrimidin-4-amine (0.53 g, 66%).

Step D:

A vial was charged with 8-bromo-N,N-bis(4-methoxybenzyl)-pyrido[4,3-d]pyrimidin-4-amine (0.80 g, 1.72 mmol), 1,1′-bis(diphenylphosphino)ferrocene palladium (II) chloride (0.17 g, 0.21 mmol), DMF (4.2 mL) and methanol (2.1 mL) (DMF and methanol were pre-degassed). The vial was purged with CO (g) for 30 seconds and the reaction mixture stirred at reflux under a CO (g) atmosphere for 16 hours. The mixture was then concentrated under reduced pressure and the crude product then purified by flash chromatography using 20-40% (20% MeOH/THF)/heptane gradient to afford methyl 4-(bis(4-methoxybenzyl)amino)pyrido[4,3-d]pyrimidine-8-carboxylate (0.44 g, 58%).

Step E:

To a solution of methyl 4-(bis(4-methoxybenzyl)amino)pyrido[4,3-d]pyrimidine-8-carboxylate (0.44 g, 0.99 mmol) in THF (2.50 mL) and water (2.50 mL) was added lithium hydroxide monohydrate (0.05 g, 1.29 mmol), and the reaction mixture was stirred at room temperature for 1 hour. 50% aqueous acetic acid was added, and the mixture was concentrated under reduced pressure. Water was then added resulting in precipitation of a solid. This residue was filtered and dried to afford 4-(bis(4-methoxybenzyl)amino)pyrido[4,3-d]pyrimidine-8-carboxylic acid (0.33 g, 77%). m/z (ES-MS) M+1=431.2. ¹H NMR (400 MHz, DMSO) δ 16.03 (s, 1H), 9.22 (s, 1H), 9.20 (s, 1H), 8.87 (s, 1H), 7.33-7.28 (m, 4H), 6.97-6.92 (m, 4H), 5.10 (s, 4H), 3.75 (s, 6H).

### Example AZ

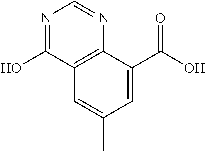

### 4-Hydroxy-6-methylquinazoline-8-carboxylic acid

Step A:

A suspension of 2-amino-3-bromo-5-methylbenzoic acid (3.50 g, 15.0 mmol) and formamidine acetate (4.88 g, 46.8 mmol) in anhydrous ethanol (20.0 mL) was heated at 80° C. for 48 hours. After cooling to room temperature, the solid was filtered and dried under high vacuum to afford 8-bromo-6-methylquinazolin-4-ol (3.48 g, 99%). ¹H NMR (500 MHz, DMSO-d₆) δ 12.42 (s, 1H), 8.12 (s, 1H), 8.00 (s, 1H), 7.93 (s, 1H), 2.40 (s, 3H). LC/MS: m/z 239.0 [M+1].

Step B:

8-Bromo-6-methylquinazolin-4-ol (7.00 g, 29.3 mmol), [1,1′-bis(diphenyl-phosphino)ferrocene]dichloropalladium(H) complex with dichloromethane (1:1) (598 mg, 0.73 mmol), triethylamine (20.4 mL, 146 mmol), and methanol (60 mL) were combined in an autoclave fitted with a large stir bar. The mixture was purged with nitrogen for five minutes. The vessel was placed under an atmosphere of carbon monoxide (300 psi) and heated to 120° C. for 18 hours. The vessel was cooled to room temperature, and the reaction mixture concentrated under reduced pressure. MeOH/1N aq. NaOH (50/50) was then added, and, after stirring at room temperature for 3 hours, the mixture was filtered and the filtrate adjusted to pH 4 by adding 10% HCl. The resulting solid was filtered and dried under high vacuum to afford 4-hydroxy-6-methylquinazoline-8-carboxylic acid (4.53 g, 76%). LC/MS: m/z 205.1 [M+1].

### Example BA

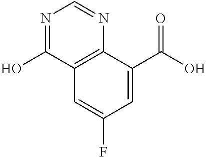 [SMILES: O=C(O)c1cc(F)cc2c(O)ncnc12]

### 4-Hydroxy-6-fluoroquinazoline-8-carboxylic acid

Step A:

Using a similar procedure as for Example AZ, Step A, using 2-amino-3-bromo-5-fluorobenzoic acid in place of 2-amino-3-bromo-5-methylbenzoic acid, 8-bromo-6-fluoroquinazolin-4-ol was obtained (840 mg, 54%). ¹H NMR (500 MHz, DMSO-d6) δ 12.54 (s, 1H), 8.29-8.11 (m, 2H), 7.85 (m, 1H). LC/MS: m/z 244.9 [M+1].

Step B:

Using a similar procedure as for Example AZ, Step B, using 8-bromo-6-fluoroquinazolin-4-ol in place of 8-bromo-6-methylquinazolin-4-ol afforded 6-fluoro-4-hydroxyquinazoline-8-carboxylic acid (700 mg, 56%). ¹H NMR (500 MHz, DMSO-d₆) δ 14.92 (bs, 1H), 13.03 (bs, 1H), 8.45 (s, 1H), 8.29-8.14 (m, 1H), 8.07 (m, 1H). LC/MS: m/z 209.0 [M+1].

### Example BB

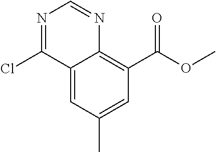 [SMILES: COC(=O)c1cc(C)cc2c(Cl)ncnc12]

### Methyl 4-chloro-6-methylquinazoline-8-carboxylate

Step A:

8-Bromo-6-methylquinazolin-4-ol (2.00 g, 8.36 mmol), [1,1′-bis(diphenyl-phosphino)ferrocene]dichloropalladium(II) complex with dichloromethane (1:1) (171.0 mg, 0.209 mmol), triethylamine (5.83 mL, 41.80 mmol), and methanol (17 mL) were combined in an autoclave. The mixture was purged with nitrogen for five minutes. The vessel was placed under an atmosphere of carbon monoxide (300 psi) and heated to 120° C. for 3 hours. The vessel was cooled to room temperature, and the reaction mixture was filtered. The collected solids were washed with methanol (250 mL). The solids were air-dried to give methyl 4-hydroxy-6-methylquinazoline-8-carboxylate (1.350 g, 74%). ¹H NMR (500 MHz, DMSO-d₆) δ 12.30 (s, 1H), 8.08 (d, 2H), 7.81 (s, 1H), 3.86 (s, 3H), 2.46 (s, 3H). LC/MS: m/z 219.0 (100%) [M+1].

Step B:

Methyl 4-hydroxy-6-methylquinazoline-8-carboxylate (1.250 g, 5.73 mmol) was dissolved in phosphoryl chloride (16.0 mL, 172 mmol) and heated to reflux for 2 hours. The mixture was stirred at room temperature overnight. The phosphoryl chloride was distilled off, and the solids were neutralized with a mixture of aqueous sodium bicarbonate solution and ice. The resulting suspension was filtered to give a solid, which was triturated with anhydrous ether. The resulting suspension was filtered to yield methyl 4-chloro-6-methylquinazoline-8-carboxylate as a solid (1.01 g, 75%). ¹H NMR (500 MHz, DMSO-d6) δ 9.10 (s, 1H), 8.31-8.16 (m, 2H), 3.98-3.88 (s, 3H), 2.62 (s, 3H). LC/MS: m/z 237.0 (100%) [M+1].

### Example 1

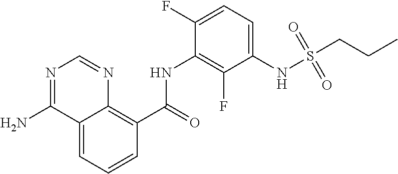 [SMILES: CCCS(=O)(=O)Nc1ccc(F)c(NC(=O)c2cccc3c(N)ncnc23)c1F]

### 4-Amino-quinazoline-8-carboxylic acid [2,6-difluoro-3-(propane-1-sulfonylamino)-phenyl]-amide

Step A:

To a solution of N-(3-amino-2,4-difluorophenyl)propane-1-sulfonamide (170 mg, 0.679 mmol) in chloroform (3 mL) was added magnesium sulfate (150 mg) and pyridine (0.16 mL, 2.04 mmol). A suspension of 4-chloroquinazoline-8-carbonyl chloride (0.20 g, 0.88 mmol) in chloroform (4 mL) was then added at room temperature. The reaction mixture was heated at 60° C. for 1 hour, and the magnesium sulfate was removed by filtration. The filtrate was diluted with dichloromethane and washed with a saturated solution of NaHCO₃. The aqueous layer was extracted twice with dichloromethane and the combined organic layers dried with sodium sulfate, filtered and concentrated in vacuo. The crude product was purified by flash chromatography to afford 4-chloro-N-(2,6-difluoro-3-(propylsulfonamido)phenyl)quinazoline-8-carboxamide (145 mg, 48%).

Step B:

In a microwave vessel, 4-chloro-N-(2,6-difluoro-3-(propylsulfonamido)-phenyl)quinazoline-8-carboxamide (0.08 g, 0.18 mmol) was dissolved in a 2M ammonia solution in isopropanol (4 mL) and heated in a microwave reactor at 105° C. for 15 minutes. The reaction mixture was concentrated in vacuo and the crude product then purified by SFC to afford 4-amino-quinazoline-8-carboxylic acid [2,6-difluoro-3-(propane-1-sulfonylamino)-phenyl]-amide (55 mg, 71%) as a solid. ¹H NMR (400 MHz, DMSO-d₆) δ 13.25 (s, 1H), 9.67 (s, 1H), 8.65 (d, J=6.6 Hz, 1H), 8.57 (s, 1H), 8.53 (d, J=8.1, 1H), 8.33 (s, 2H), 7.68 (t, J=7.8 Hz, 1H), 7.37 (dd, J=14.4, 8.6 Hz, 1H), 7.22 (t, J=8.6 Hz, 1H), 3.14-3.01 (m, 2H), 1.82-1.69 (m, 2H), 0.98 (t, J=7.4 Hz, 3H). m/z (ES-MS) 422.1 [M+1].

### Example 2

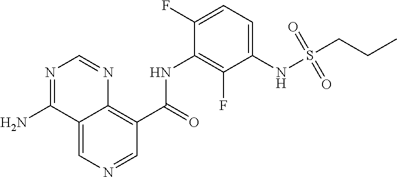 [SMILES: CCCS(=O)(=O)Nc1ccc(F)c(NC(=O)c2cncc3c(N)ncnc23)c1F]

### 4-Amino-pyrido[4,3-d]pyrimidine-8-carboxylic acid [2,6-difluoro-3-(propane-1-sulfonylamino)-phenyl]-amide

Step A:

To a solution of 4-(bis(4-methoxybenzyl)amino)pyrido[4,3-d]pyrimidine-8-carboxylic acid (0.32 g, 0.73 mmol) in DCM (7.9 mL) at 0° C. was added a solution of oxalyl chloride (0.07 mL, 0.81 mmol) in DCM (0.25 mL). The reaction mixture was stirred at 0° C. for 15 minutes and then warmed to room temperature for 10 minutes. The mixture was concentrated in vacuo and the residue dissolved in chloroform (7.3 mL). N-(3-amino-2,4-difluorophenyl)propane-1-sulfonamide (0.21 g, 0.84 mmol) was then added, and the mixture was stirred at 60° C. for 45 minutes. The reaction was concentrated in vacuo and the residue purified by flash chromatography (0-5% MeOH/DCM) to afford 4-(bis(4-methoxybenzyl)amino)-N-(2,6-difluoro-3-(propylsulfonamido)phenyl)pyrido[4,3-d]pyrimidine-8-carboxamide (0.35 g, 72%).

Step B:

A solution of 4-(bis(4-methoxybenzyl)amino)-N-(2,6-difluoro-3-(propylsulfonamido)phenyl)pyrido[4,3-d]pyrimidine-8-carboxamide (112 mg, 0.17 mmol) in TFA (2.50 mL) was heated at 70° C. for 30 minutes and the TFA was then removed in vacuo. The residue was re-dissolved in TFA (2.50 mL) in a microwave vessel and the mixture heated to 115° C. in a microwave reactor for 60 minutes. The reaction mixture was concentrated in vacuo and the residue purified by reverse phase HPLC and SFC to afford 4-amino-pyrido[4,3-d]pyrimidine-8-carboxylic acid [2,6-difluoro-3-(propane-1-sulfonylamino)-phenyl]-amide (51 mg, 71%), as a solid. ¹H NMR (400 MHz, DMSO-d₆) δ 12.60 (s, 1H), 9.74 (s, 1H), 9.72 (s, 1H), 9.41 (s, 1H), 8.94 (s, 1H), 8.70 (s, 1H), 8.69 (s, 1H), 7.39 (td, J=8.9, 5.7 Hz, 1H), 7.24 (t, J=8.5 Hz, 1H), 3.09 (dd, J=8.7, 6.6 Hz, 2H), 1.81-1.69 (m, 2H), 0.98 (t, J=7.4 Hz, 3H). m/z (ES-MS) 423.0 [M+1].

### Example 3

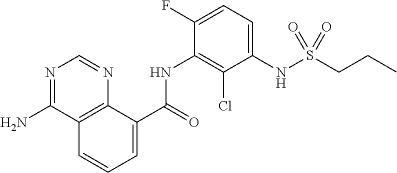 [SMILES: CCCS(=O)(=O)Nc1ccc(F)c(NC(=O)c2cccc3c(N)ncnc23)c1Cl]

### 4-Amino-quinazoline-8-carboxylic acid [2-chloro-6-fluoro-3-(propane-1-sulfonylamino)-phenyl]-amide

Step A:

N-(3-Amino-2-chloro-4-fluorophenyl)propane-1-sulfonamide (500 mg, 1.87 mmol) was dissolved in chloroform (10 mL), and 4 Å molecular sieves (800 mg), pyridine (0.152 mL, 1.87 mmol), and 4-chloroquinazoline-8-carbonyl chloride (851 mg, 3.75 mmol) were added in the indicated order. The reaction mixture was stirred at room temperature for 1-2 hours and filtered. The filtrate was washed with saturated aqueous NaHCO₃ solution and brine and then dried over MgSO₄. After removing the solvent under reduced pressure, the crude product was purified using flash chromatography (gradient elution, solvent: 0-15% ethyl acetate in dichloromethane) to yield 4-chloro-N-(2-chloro-6-fluoro-3-(propylsulfonamido)phenyl)-quinazoline-8-carboxamide (652 mg, 76%). ¹H NMR (400 MHz, DMSO-d₆) δ 12.00 (s, 1H), 9.64 (s, 1H), 9.29 (s, 1H), 8.85 (dd, 1H), 8.61 (dt, 1H), 8.07 (dd, 1H), 7.50 (dd, 1H), 7.41 (t, 1H), 3.17-3.07 (m, 2H), 1.89-1.68 (m, 2H), 1.08-0.89 (m, 3H).

Step B:

4-Chloro-N-(2-chloro-6-fluoro-3-(propylsulfonamido)phenyl)quinazoline-8-carboxamide was suspended in isopropanol (5 mL) in a microwave vial (10-20 mL) and saturated with ammonia for 15 minutes. The reaction mixture was then heated in a microwave reactor at 105° C. for 15 minutes. After removal of the solvent under reduced pressure, the crude product was purified by HPLC to yield 4-amino-quinazoline-8-carboxylic acid [2-chloro-6-fluoro-3-(propane-1-sulfonylamino)-phenyl]-amide as a solid (50.0 mg, 68%). ¹H NMR (400 MHz, DMSO-d₆) δ 13.25 (s, 1H), 9.68 (s, 1H), 8.69-8.61 (m, 1H), 8.57 (s, 1H), 8.56-8.49 (m, 1H), 8.32 (s, 2H), 7.68 (t, J=7.8 Hz, 1H), 7.37 (td, J=8.8, 5.7 Hz, 1H), 7.22 (t, J=8.7 Hz, 1H), 3.15-3.03 (m, 2H), 1.84-1.68 (m, 2H), 1.04 (d, J=6.1 Hz, 1H), 1.02-0.93 (m, 3H). LC/MS: m/z 438.0 [M+1].

### Example 4

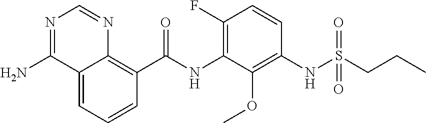 [SMILES: CCCS(=O)(=O)Nc1ccc(F)c(NC(=O)c2cccc3c(N)ncnc23)c1OC]

### 4-Amino-quinazoline-8-carboxylic acid [6-fluoro-2-methoxy-3-(propane-1-sulfonylamino)-phenyl]-amide

Step A:

A 100 mL round bottom flask was charged with N-(3-amino-4-fluoro-2-methoxyphenyl)-N-(4-methoxybenzyl)propane-1-sulfonamide (108 mg, 0.282 mmol) dissolved in chloroform (3 mL, 30 mmol). To this mixture was added triethylamine (86.6 uL, 0.621 mmol) and then 4-chloroquinazoline-8-carbonyl chloride (83.4 mg, 0.367 mmol), and the reaction mixture was heated to 60° C. for 3.5 hours. After removal of the solvent under reduced pressure, the crude product was purified by flash chromatography using 20-70% ethyl acetate: heptanes to give 4-chloro-N-(6-fluoro-2-methoxy-3-(N-(4-methoxybenzyl)propylsulfonamido)phenyl)quinazoline-8-carboxamide as a solid (88 mg, 55%). ¹H NMR (400 MHz, DMSO-d₆) δ=11.59 (s, 1H), 9.31 (s, 1H), 8.79 (d, J=6.0 Hz, 1H), 8.64-8.55 (m, 1H), 8.12-8.02 (m, 1H), 7.17 (d, J=8.6 Hz, 2H), 7.11 (dd, J=9.0, 6.0 Hz, 1H), 7.01 (t, J=9.0 Hz, 1H), 6.85 (d, J=8.6 Hz, 2H), 4.69 (s, 2H), 3.86 (s, 3H), 3.71 (s, 3H), 3.25 (d, J=8.4 Hz, 2H), 1.80 (dt, J=16.6, 8.4 Hz, 2H), 1.02 (t, J=7.4 Hz, 3H).

Step B:

A microwave vial was charged with 4-chloro-N-(6-fluoro-2-methoxy-3-(N-(4-methoxybenzyl)propylsulfonamido)phenyl)quinazoline-8-carboxamide (87 mg, 0.15 mmol) dissolved in 1,4-dioxane (1 mL, 10 mmol), and this solution was saturated with ammonia gas. The reaction mixture was heated in a microwave reactor to 120° C. for 20 minutes and then concentrated to remove the 1,4-dioxane. The residue was stirred with 2 mL of water at reflux for one minute and then at room temperature for 30 minutes. The water was discarded. The residual oil was dissolved into methanol and concentrated to dryness to give 4-amino-N-(6-fluoro-2-methoxy-3-(N-(4-methoxybenzyl)propylsulfonamido)-phenyl)quinazoline-8-carboxamide as a oil. ¹H NMR (400 MHz, DMSO-d₆) δ=11.59 (s, 1H), 9.31 (s, 1H), 8.79 (d, J=6.0 Hz, 1H), 8.64-8.55 (m, 1H), 8.12-8.02 (m, 1H), 7.17 (d, J=8.6 Hz, 2H), 7.11 (dd, J=9.0, 6.0 Hz, 1H), 7.01 (t, J=9.0 Hz, 1H), 6.85 (d, J=8.6 Hz, 2H), 4.69 (s, 2H), 3.86 (s, 3H), 3.71 (s, 3H), 3.25 (d, J=8.4 Hz, 2H), 1.80 (dt, J=16.6, 8.4 Hz, 2H), 1.02 (t, J=7.4 Hz, 3H).

Step C:

4-Amino-N-(6-fluoro-2-methoxy-3-(N-(4-methoxybenzyl)propyl-sulfonamido)phenyl)-quinazoline-8-carboxamide was dissolved in methylene chloride (5 mL, 80 mmol) and trifluoroacetic acid (5 mL, 60 mmol) was added followed by stirring at room temperature for 30 minutes. Following removal of the dichloromethane and trifluoroacetic acid under reduced pressure, the material was redissolved in dichloromethane (15 mL) and concentrated to dryness. The crude product was triturated with 10 mL of hot ethyl ether and the residue recrystallized from 4 mL hot ethyl acetate, filtered and dried in the vacuum oven at 80° C. for 60 hours to yield 4-amino-quinazoline-8-carboxylic acid [6-fluoro-2-methoxy-3-(propane-1-sulfonylamino)-phenyl]-amide as a solid (28 mg, 42%). ¹H NMR (400 MHz, DMSO-d₆) δ=13.09 (s, 1H), 9.21 (s, 1H), 8.63 (dd, J=26.3, 13.5 Hz, 4H), 7.75 (s, 1H), 7.41-7.23 (m, 1H), 7.09 (t, J=9.2 Hz, 1H), 3.81 (s, 3H), 3.17-3.05 (m, 2H), 1.77 (dd, J=15.1, 7.5 Hz, 2H), 0.99 (t, J=7.4 Hz, 3H). MS m/z 434.3 [M+1].

### Example 5

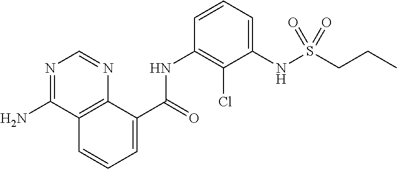 [SMILES: CCCS(=O)(=O)Nc1cccc(NC(=O)c2cccc3c(N)ncnc23)c1Cl]

### 4-Amino-quinazoline-8-carboxylic acid [2-chloro-3-(propane-1-sulfonylamino)-phenyl]-amide

Step A:

4-(2,4-Dimethoxybenzylamino)quinazoline-8-carboxylic acid (0.0390 g, 0.115 mmol), N-(3-amino-2-chlorophenyl)propane-1-sulfonamide (0.022 g, 0.0884 mmol), HATU (0.0437 g, 0.115 mmol) and DIEA (d 0.742) (0.0308 mL, 0.177 mmol) were dissolved in DMF and stirred at 55° C. overnight. The reaction mixture was cooled to room temperature, partitioned between EtOAc and water and the layers separated. The organic layer was washed with water (3×), 0.1 N HCl, saturated aqueous NaHCO₃ and brine, dried over Na₂SO₄ and concentrated to an oil. The oil was filtered through a plug of SiO₂ with the aid of 2:1 Hex/EtOAc to give N-(2-chloro-3-(propylsulfonamido)phenyl)-4-(2,4-dimethoxybenzyl-amino)quinazoline-8-carboxamide as an oil which was used directly in the next step.

Step B:

N-(2-chloro-3-(propylsulfonamido)phenyl)-4-(2,4-dimethoxybenzylamino)-quinazoline-8-carboxamide (0.0352 g, 0.0617 mmol) was dissolved in TFA (5 mL) and heated at reflux for 3 hours. The reaction mixture was cooled to room temperature and concentrated to an oil. The residue was dissolved in EtOAc, washed with saturated aqueous NaHCO₃ and brine, dried over Na₂SO₄ and concentrated to an oil. DCM was added, and a precipitate formed which was collected by filtrations, washed further with DCM and dried under high vacuum to give 4-amino-quinazoline-8-carboxylic acid [2-chloro-3-(propane-1-sulfonylamino)-phenyl]-amide (9 mg, 0.0214 mmol, 35%). ¹H NMR (400 MHz, DMSO-d₆) δ 9.53 (br s, 1H), 8.72-8.74 (d, 1H), 8.67 (s, 1H), 8.51-8.55 (t, 1H), 8.24-8.41 (br s, 2H), 7.68-7.72 (t, 1H), 7.36-7.40 (t, 1H), 7.24-7.26 (t, 1H), 3.13-3.16 (t, 1H), 1.76-1.82 (m, 2H), 0.97-1.01 (t, 3H); m/z (APCI-pos) M+1=420.1, 422.1.

### Example 6

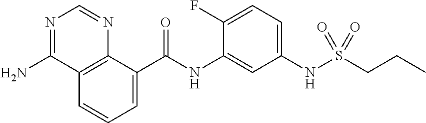 [SMILES: CCCS(=O)(=O)Nc1ccc(F)c(NC(=O)c2cccc3c(N)ncnc23)c1]

### 4-Amino-quinazoline-8-carboxylic acid [2-fluoro-5-(propane-1-sulfonylamino)-phenyl]-amide

Step A:

A mixture of 4-(2,4-dimethoxyamino)quinazoline-8-carboxylic acid (52 mg, 0.15 mmol), N-(3-amino-4-fluorophenyl)-propane-1-sulfonamide (36 mg, 0.15 mmol), HATU (65 mg, 0.17 mmol), N,N-diisopropylethylamine (67 ul, 0.39 mmol), and a catalytic amount of 4-dimethylaminopyridine (“DMAP”) (1.9 mg, 0.015 mmol) in DMF (1.5 mL) was stirred at room temperature for 1 hour. The reaction mixture was diluted with ethyl acetate (50 mL). The organic phase was washed with brine and removed under reduced pressure to give 4-(2,4-dimethoxybenzylamino)-N-(2-fluoro-5-(propylsulfonamido)phenyl)quinazoline-8-carboxamide (86 mg, 99%), which was used in the next step without further purification. LC-MS [M+1] m/z 554.1.

Step B:

4-(2,4-Dimethoxy-benzylamino)-N-(2-fluoro-5-(propylsulfonamido)phenyl)quinazoline-8-carboxamide (86 mg, 0.155 mmol) was taken up in trifluoroacetic acid (“TFA”) (4 mL). The reaction mixture was refluxed for 2 hours. The solvent was removed under reduced pressure and the mixture purified by preparative HPLC to afford 4-amino-quinazoline-8-carboxylic acid [2-fluoro-5-(propane-1-sulfonylamino)-phenyl]amide (38 mg, 61%). ¹H NMR (500 MHz, DMSO-d₆) δ 14.15 (s, 1H), 9.73 (s, 1H), 8.72 (d, J=7.4 Hz, 1H), 8.58 (s, 2H), 8.32 (s, 2H), 7.69 (t, J=7.8 Hz, 1H), 7.41-7.17 (m, 1H), 6.98 (dd, J=8.1, 3.7 Hz, 1H), 3.11-2.95 (m, 2H), 1.71 (dd, J=15.1, 7.5 Hz, 2H), 0.95 (s, 3H). LC-MS [M+1] m/z 404.1.

### Example 7

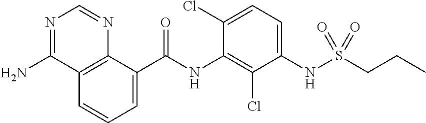 [SMILES: CCCS(=O)(=O)Nc1ccc(Cl)c(NC(=O)c2cccc3c(N)ncnc23)c1Cl]

### 4-Amino-quinazoline-8-carboxylic acid [2,6-dichloro-3-(propane-1-sulfonylamino)-phenyl]-amide

Step A:

To N-(3-amino-2,4-dichlorophenyl)propane-1-sulfonamide (151 mg, 0.533 mmol) in chloroform (3 mL), in the presence of activated 4 Å molecular sieves, was added pyridine (43 ul, 0.533 mmol) and then 4-chloro-quinazoline-8-carbonyl chloride (182 mg, 0.8 mmol). The reaction mixture was left stirring at ambient temperature for an hour. The crude product was purified by chromatography eluting with 1:1 ethyl acetate/hexane to yield 253 mg (78%) of 4-chloro-N-(2,6-dichloro-3-(propylsulfonamido)phenyl)quinazoline-8-carboxamide. LC-MS [M+1] m/z 473.0.

Step B:

4-Chloro-N-(2,6-dichloro-3-(propylsulfonamido)phenyl)quinazoline-8-carboxamide (86.3 mg, 0.176 mmol) was dissolved in isopropyl alcohol (5 mL). Ammonia gas was passed through the solution for 15 minutes. The solvent was removed under reduced pressure and the crude product was purified by preparative HPLC to afford 4-amino-quinazoline-8-carboxylic acid [2,6-dichloro-3-(propane-1-sulfonylamino)-phenyl]amide (51.3 mg, 64%) ¹H NMR (500 MHz, DMSO-d₆) δ 13.50 (s, 1H), 9.62 (s, 1H), 8.65 (d, J=7.4 Hz, 1H), 8.57 (s, 1H), 8.53 (d, J=8.3 Hz, 1H), 8.20 (d, J=63.1 Hz, 2H), 7.68 (t, J=7.8 Hz, 1H), 7.56 (d, J=9.0 Hz, 1H), 7.48 (s, 1H), 3.19-3.06 (m, 1H), 1.76 (dt, J=14.8, 7.4 Hz, 1H), 0.98 (t, J=7.4 Hz, 2H). LC-MS [M+1] m/z 454.0.

Examples 8-27 in Table 1 were prepared according to the above examples using appropriate starting materials.

TABLE 1

| | | | | ¹H NMR δ |
|---|---|---|---|---|
| Example | | | MS m/z | (400 MHz, DMSO-d6*, |
| no. | Structure | Name | [M + H]⁺ | CDCl₃**, or CD₃OD***) |
| 8 | 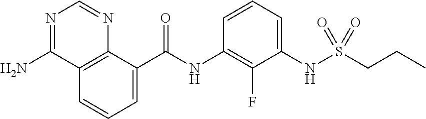 [SMILES: CCCS(=O)(=O)Nc1cccc(NC(=O)c2cccc3c(N)ncnc23)c1F] | 4-Amino- quinazoline-8- carboxylic acid [2- fluoro-3- (propane-1- sulfonylamino)- phenyl]-amide | 404.1 | *14.09 (s, 1H), 9.68 (s, 1H), 8.71 (dd, J = 7.5, 1.3 Hz, 1H), 8.57 (s, 2H), 8.33 (t, J = 6.4 Hz, 3H), 7.69 (s, 1H), 7.17 (s, 2H), 3.26-3.01 (m, 2H), 1.77 (d, J = 7.6 Hz, 2H), 1.00 (t, J = 7.4 Hz, 3H) |
| 9 | 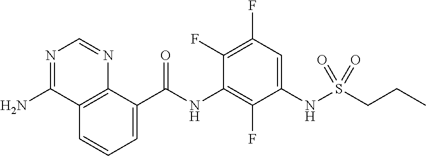 [SMILES: CCCS(=O)(=O)Nc1cc(F)c(F)c(NC(=O)c2cccc3c(N)ncnc23)c1F] | 4-Amino- quinazoline-8- carboxylic acid [2,3,6- trifluoro-5- (propane-1- sulfonylamino)- phenyl]-amide | 440.1 | *13.46 (s, 1H), 9.93 (s, 1H), 8.65 (s, 1H), 8.57 (s, 2H), 8.33 (s, 2H), 7.69 (s, 1H), 7.46 (s, 1H), 3.16 (s, 2H), 1.75 (s, 2H), 0.98 (s, 3H) |
| 10 | 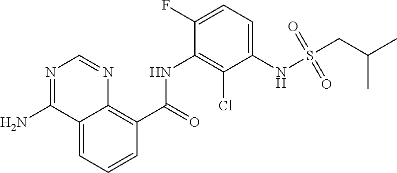 [SMILES: CC(C)CS(=O)(=O)Nc1ccc(F)c(NC(=O)c2cccc3c(N)ncnc23)c1Cl] | 4-Amino- quinazoline-8- carboxylic acid [2- chloro-6- fluoro-3-(2- methyl-propane-1- sulfonylamino)- phenyl]-amide | 452.1 | *13.45 (s, 1H), 9.55 (s, 1H), 8.69-8.60 (m, 1H), 8.58 (s, 1H), 8.53 (d, J = 8.3, 1H), 8.30 (s, 2H), 7.68 (t, J = 7.8, 1H), 7.45 (dd, J = 9.1, 5.2, 1H), 7.36 (t, J = 9.1, 1H), 3.05 (d, J = 6.5, 2H), 2.21 (dt, J = 13.3, 6.7, 1H), 1.04 (d, J = 6.7, 6H) |
| 11 | 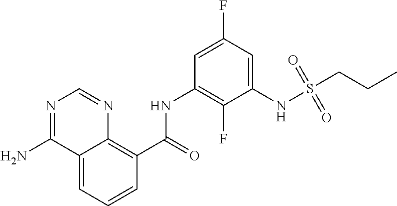 [SMILES: CCCS(=O)(=O)Nc1cc(F)cc(NC(=O)c2cccc3c(N)ncnc23)c1F] | 4-Amino- quinazoline-8- carboxylic acid [2,5- difluoro-3- (propane-1- sulfonylamino)- phenyl]-amide | 422.1 | *14.38 (s, 1H), 10.00 (s, 1H), 8.72 (d, J = 7.3 Hz, 1H), 8.57 (s, 1H), 8.54 (d, J = 8.0 Hz, 1H), 8.41 (s, 2H), 8.22-8.14 (m, 1H), 7.71 (t, J = 7.8 Hz, 1H), 7.01 (ddd, J = 9.4, 5.9, 3.2 Hz, 1H), 3.23-3.15 (m, 2H), 1.83-1.68 (m, 2H), 1.00 (t, J = 7.4 Hz, 3H) |
| 12 | 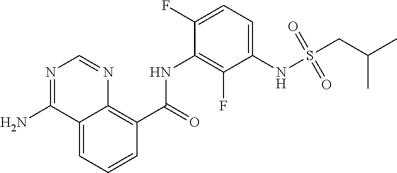 [SMILES: CC(C)CS(=O)(=O)Nc1ccc(F)c(NC(=O)c2cccc3c(N)ncnc23)c1F] | 4-Amino- quinazoline-8- carboxylic acid [2,6- difluoro-3- (2-methyl- propane-1- sulfonylamino)- phenyl]-amide | 436.1 | *13.25 (s, 1H), 9.69 (s, 1H), 8.67-8.60 (m, 1H), 8.57 (s, 1H), 8.53 (d, J = 7.0 Hz, 1H), 8.32 (s, 2H), 7.68 (t, J = 7.8 Hz, 1H), 7.36 (td, J = 8.8, 5.7 Hz, 1H), 7.22 (t, J = 9.2 Hz, 1H), 3.01 (d, J = 6.4 Hz, 2H), 2.20 (td, J = 13.0, 6.4 Hz, 1H), 1.03 (d, J = 6.7 Hz, 6H) |
| 13 | 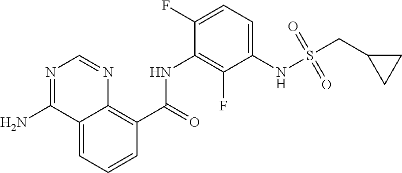 [SMILES: Nc1ncnc2c(C(=O)Nc3c(F)ccc(NS(=O)(=O)CC4CC4)c3F)cccc12] | 4-Amino- quinazoline-8- carboxylic acid (3- cyclopropyl- methane- sulfonylamino- 2,6-difluoro- phenyl)-amide | 434.1 | *13.23 (s, 1H), 9.70 (s, 1H), 8.68-8.60 (m, 1H), 8.57 (s, 1H), 8.53 (d, J = 7.0 Hz, 1H), 8.32 (s, 2H), 7.68 (t, J = 7.8 Hz, 1H), 7.39 (td, J = 8.8, 5.7 Hz, 1H), 7.20 (t, J = 9.2 Hz, 1H), 3.09 (d, J = 7.1 Hz, 2H), 1.12- 1.02 (m, 1H), 0.60-0.52 (m, 2H), 0.35 (q, J = 4.6 Hz, 2H) |
| 14 | 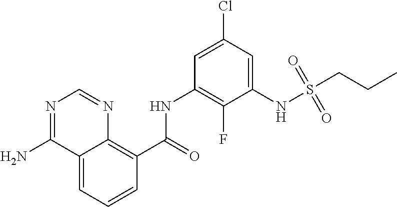 [SMILES: CCCS(=O)(=O)Nc1cc(Cl)cc(NC(=O)c2cccc3c(N)ncnc23)c1F] | 4-Amino- quinazoline-8- carboxylic acid [5- chloro-2- fluoro-3- (propane-1- sulfonylamino)- phenyl]-amide | 438.0 | *14.37 (s, 1H), 9.98 (s, 1H), 8.71 (d, J = 7.5 Hz, 1H), 8.57 (s, 1H), 8.53 (d, J = 8.2 Hz, 0H), 8.45-8.40 (m, 1H), 8.28 (s, 2H), 7.70 (t, J = 7.9 Hz, 1H), 7.21 (dd, J = 6.5, 2.6 Hz, 1H), 3.22-3.14 (m, 2H), 1.76 (sx, J = 7.4 Hz, 2H), 1.00 (t, J = 7.4 Hz, 3H) |
| 15 | 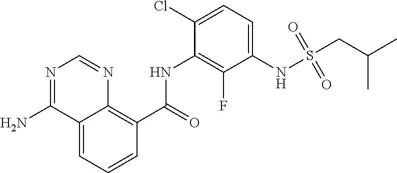 [SMILES: CC(C)CS(=O)(=O)Nc1ccc(Cl)c(NC(=O)c2cccc3c(N)ncnc23)c1F] | 4-Amino- quinazoline-8- carboxylic acid [6- chloro-2- fluoro-3-(2- methyl-propane-1- sulfonylamino)- phenyl]-amide | 452.0 | *13.46 (s, 1H), 9.89 (s, 1H), 8.65 (d, J = 7.5 Hz, 1H), 8.58 (s, 1H), 8.54 (d, J = 8.2 Hz, 1H), 8.33 (s, 2H), 7.69 (t, J = 7.7 Hz, 1H), 7.48-7.34 (m, 2H), 3.05 (d, J = 6.3 Hz, 2H), 2.18 (dt, J = 13.1, 6.5 Hz, 1H), 1.03 (d, J = 6.7 Hz, 6H) |
| 16 | 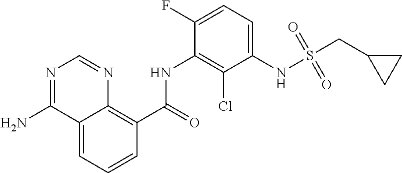 [SMILES: Nc1ncnc2c(C(=O)Nc3c(F)ccc(NS(=O)(=O)CC4CC4)c3Cl)cccc12] | 4-Amino- quinazoline-8- carboxylic acid (2- chloro-3- cyclopropyl- methane- sulfonylamino- 6-fluoro- phenyl)-amide | 450.0 | *13.47 (s, 1H), 9.55 (s, 1H), 8.65 (d, J = 7.3 Hz, 1H), 8.58 (s, 1H), 8.53 (d, J = 8.0 Hz, 1H), 8.32 (s, 2H), 7.68 (t, J = 7.8 Hz, 1H), 7.51-7.44 (m, 1H), 7.36 (t, J = 9.0 Hz, 1H), 3.14 (d, J = 7.1 Hz, 2H), 1.16- 1.06 (m, 1H), 0.58 (d, J = 7.6 Hz, 2H), 0.37 (d, J = 4.4 Hz, 2H) |
| 17 | 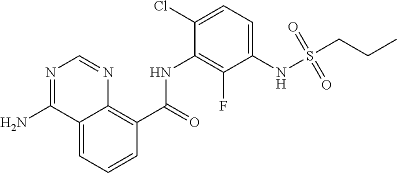 [SMILES: CCCS(=O)(=O)Nc1ccc(Cl)c(NC(=O)c2cccc3c(N)ncnc23)c1F] | 4-Amino- quinazoline-8- carboxylic acid [6- chloro-2- fluoro-3- (propane-1- sulfonylamino)- phenyl]-amide | 438.0 | *13.47 (s, 1H), 9.90 (s, 1H), 8.65 (dd, J = 7.5, 1.4 Hz, 1H), 8.58 (s, 1H), 8.53 (dd, J = 8.3, 1.4 Hz, 1H), 8.43 (s, 1H), 8.26 (s, 1H), 7.69 (t, J = 7.9 Hz, 1H), 7.47-7.36 (m, 2H), 3.16-3.08 (m, 2H), 1.75 (sx, J = 7.4 Hz, 2H), 0.97 (t, J = 7.4 Hz, 3H) |
| 18 | 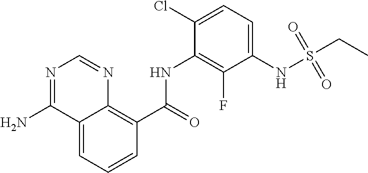 [SMILES: CCS(=O)(=O)Nc1ccc(Cl)c(NC(=O)c2cccc3c(N)ncnc23)c1F] | 4-Amino- quinazoline-8- carboxylic acid (6- chloro-3- ethanesulfonyl- amino-2- fluoro-phenyl)- amide | 424.0 | *13.47 (s, 1H), 9.90 (s, 1H), 8.65 (d, J = 6.6 Hz, 1H), 8.58 (s, 1H), 8.53 (d, J = 8.2 Hz, 1H), 8.42 (s, 1H), 8.26 (s, 1H), 7.69 (t, J = 7.8 Hz, 1H), 7.47- 7.35 (m, 2H), 3.15 (q, J = 7.3 Hz, 2H), 1.26 (t, J = 7.3 Hz, 3H) |
| 19 | 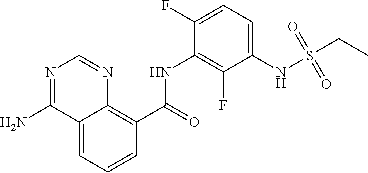 [SMILES: CCS(=O)(=O)Nc1ccc(F)c(NC(=O)c2cccc3c(N)ncnc23)c1F] | 4-Amino- quinazoline-8- carboxylic acid (3- ethanesulfonyl- amino- 2,6-difluoro- phenyl)- amide | 408.0 | *13.25 (s, 1H), 9.68 (s, 1H), 8.65 (dd, J = 7.5, 1.2 Hz, 1H), 8.57 (s, 1H), 8.53 (dd, J = 8.2, 1.3 Hz, 1H), 8.32 (s, 2H), 7.68 (t, J = 7.8 Hz, 1H), 7.37 (td, J = 8.8, 5.6 Hz, 1H), 7.22 (t, J = 8.7 Hz, 1H), 3.11 (q, J = 7.3 Hz, 2H), 1.27 (t, J = 7.3 Hz, 3H) |
| 20 | 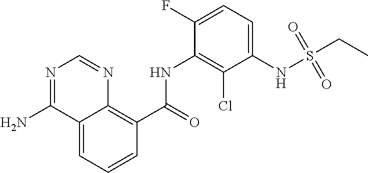 [SMILES: CCS(=O)(=O)Nc1ccc(F)c(NC(=O)c2cccc3c(N)ncnc23)c1Cl] | 4-Amino- quinazoline-8- carboxylic acid (2- chloro-3- ethanesulfonyl- amino-6- fluoro-phenyl)- amide | 424.1 | *13.49 (s, 1H), 9.62 (s, 1H), 8.74-8.48 (m, 3H), 8.33 (d, 2H), 7.69 (m, 1H), 7.53-7.29 (m, 2H), 3.16 (q, 2H), 1.30 (t, 3H) |
| 21 | 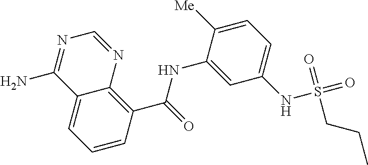 [SMILES: CCCS(=O)(=O)Nc1ccc(C)c(NC(=O)c2cccc3c(N)ncnc23)c1] | 4-Amino- quinazoline-8- carboxylic acid [2- methyl-5- (propane-1 sulfonylamino)- phenyl]-amide | 400.2 | **13.53-13.57 (bs, 1H), 8.96- 9.00 (m, 1H), 8.71 (s, 1H), 8.36-8.38 (m, 1H), 7.94-7.98 (m, 1H), 7.66-7.71 (m, 1H), 7.20-7.23 (m, 1H), 7.09-7.13 (m, 1H), 6.39-6.43 (bs, 1H), 5.84-5.89 (bs, 2H), 3.09-3.14 (m, 2H), 2.52 (s, 3H), 1.84-1.95 (m, 2H), 1.00-1.06 (m, 3H) |
| 22 | 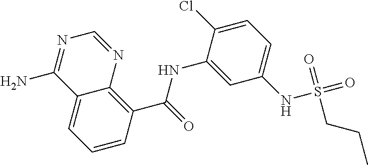 [SMILES: CCCS(=O)(=O)Nc1ccc(Cl)c(NC(=O)c2cccc3c(N)ncnc23)c1] | 4-Amino- quinazoline-8- carboxylic acid [2- chloro-5- (propane-1- sulfonylamino)- phenyl]-amide | 420.1 | ***8.82-8.85 (m, 1H), 8.58 (s, 1H), 8.56-8.58 (m, 1H), 8.35- 8.39 (m, 1H), 7.65-7.70 (m, 1H), 7.41-7.44 (m, 1H), 7.05- 7.09 (m, 1H), 3.21-3.17 (m, 2H), 2.52 (s, 3H), 1.82-1.91 (m, 2H), 1.03-1.07 (m, 3H) |
| 23 | 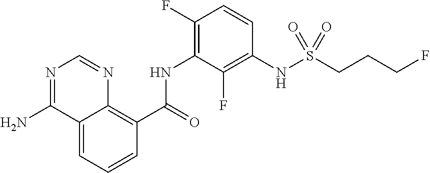 [SMILES: Nc1ncnc2c(C(=O)Nc3c(F)ccc(NS(=O)(=O)CCCF)c3F)cccc12] | 4-Amino- quinazoline-8- carboxylic acid [2,6- difluoro-3- (3-fluoro- propane-1- sulfonylamino)- phenyl]-amide | 440.2 | *13.28 (br s, 1H), 9.84 (br s, 1H), 8.64 (d, J = 7.0 Hz, 1H), 8.57 (s, 1H), 8.52 (d, J = 7.0 Hz, 1H), 8.43-8.19 (br s, 2H), 7.68 (t, J = 7.0 Hz, 1H), 7.38 (m, 1H), 7.24 (m, 1H), 4.61 (m, 1H), 4.49 (m, 1H), 3.22 (m, 2H), 2.19-2.06 (m, 2H) |
| 24 | 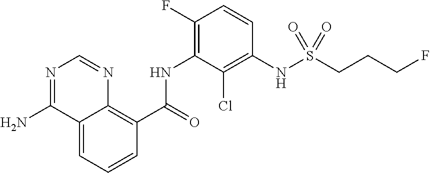 [SMILES: Nc1ncnc2c(C(=O)Nc3c(F)ccc(NS(=O)(=O)CCCF)c3Cl)cccc12] | 4-Amino- quinazoline-8- carboxylic acid [2- chloro-6- fluoro-3-(3- fluoro-propane-1- sulfonylamino)- phenyl]-amide | 456.1, 458.1 | *13.49 (br s, 1H), 9.76 (br s, 1H), 8.64 (d, J = 7.0 Hz, 1H), 8.57 (s, 1H), 8.52 (d, J = 7.0 Hz, 1H), 8.43-8.19 (br s, 2H), 7.68 (m, 1H), 7.46 (m, 1H), 7.38 (m, 1H), 4.61 (m, 1H), 4.49 (m, 1H), 3.25 (m, 2H), 2.22-2.09 (m, 2H) |
| 25 | 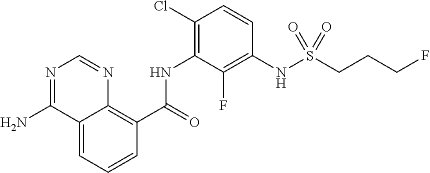 [SMILES: Nc1ncnc2c(C(=O)Nc3c(Cl)ccc(NS(=O)(=O)CCCF)c3F)cccc12] | 4-Amino- quinazoline-8- carboxylic acid [6- chloro-2- fluoro-3-(3- fluoro-propane-1- sulfonylamino)- phenyl]-amide | 456.1, 458.1 | *8.76 (d, J = 7.4 Hz, 1H), 8.57 (s, 1H), 8.41 (d, J = 7.8 Hz, 1H), 7.68 (m, 1H), 7.51 (m, 1H), 7.38 (m, 1H), 4.61 (m, 1H), 4.49 (m, 1H), 3.27 (m, 2H), 2.28-2.15 (m, 2H) |
| 26 | 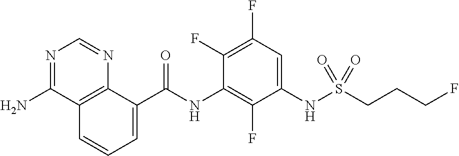 [SMILES: Nc1ncnc2c(C(=O)Nc3c(F)c(F)cc(NS(=O)(=O)CCCF)c3F)cccc12] | 4-Amino- quinazoline-8- carboxylic acid [2,3,6- trifluoro-5- (3-fluoro- propane-1- sulfonylamino)- phenyl]-amide | 458.0 | *13.49 (s, 1H), 10.09 (s, 1H), 8.65 (d, J = 7.5 Hz, 1H), 8.63- 8.49 (m, 2H), 8.34 (d, J = 62.6 Hz, 2H), 7.69 (t, J = 8.0 Hz, 1H), 7.58-7.35 (m, 1H), 4.61 (t, J = 5.9 Hz, 1H), 4.49 (t, J = 5.3 Hz, 1H), 3.26 (s, 1H), 2.11 (d, J = 29.9 Hz, 2H) |
| 27 | 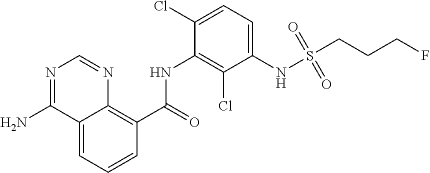 [SMILES: Nc1ncnc2c(C(=O)Nc3c(Cl)ccc(NS(=O)(=O)CCCF)c3Cl)cccc12] | 4-Amino- quinazoline-8- carboxylic acid [2,6- dichloro-3- (3-fluoro- propane-1- sulfonylamino)- phenyl]-amide | 472.0 | 13.57 (s, 1H), 9.87 (s, 1H), 8.69- 8.60 (m, 1H), 8.58 (s, 1H), 8.53 (d, J = 8.3 Hz, 1H), 8.42 (s, 1H), 8.25 (s, 1H), 7.68 (t, J = 7.9 Hz, 1H), 7.61 (d, J = 8.8 Hz, 1H), 7.48 (d, J = 8.9 Hz, 1H), 4.61 (t, J = 5.9 Hz, 1H), 4.50 (t, J = 5.9 Hz, 1H), 3.31-3.23 (m, 2H), 2.21-2.06 (m, 2H) |

### Example 28

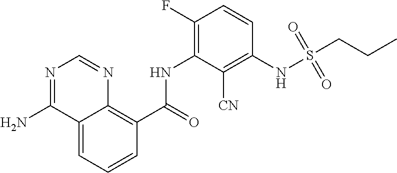 [SMILES: CCCS(=O)(=O)Nc1ccc(F)c(NC(=O)c2cccc3c(N)ncnc23)c1C#N]

### 4-Amino-quinazoline-8-carboxylic acid [2-cyano-6-fluoro-3-(propane-1-sulfonylamino)-phenyl]-amide

To 4-chloroquinazoline-8-carbonyl chloride (0.0826 g, 0.364 mmol) (Example 1, Step B) in 3 mL chloroform was added a solution of N-(3-amino-2-cyano-4-fluorophenyl)propane-1-sulfonamide (0.072 g, 0.280 mmol) in 3 mL chloroform. The mixture was stirred at 50° C. in a sealed vial for 20 h, then stirred at ambient temperature over the weekend. The reaction mixture was evaporated, the resulting yellow residue was suspended in 4 mL isopropanol and, to this, was added 7 M ammonia in methanol (0.400 mL, 2.80 mmol). The mixture was stirred at 40° C. in a sealed vial. After 1.5 hours the reaction mixture was evaporated, partitioned between water (adjusted to pH 4 with 1 M HCl) and EtOAc. The EtOAc was washed with brine, dried over MgSO₄, filtered, and evaporated to yield 115 mg yellow film. This material was absorbed on silica gel and chromatographed on as silica gel column, eluting with EtOAc to provide 4-amino-quinazoline-8-carboxylic acid [2-cyano-6-fluoro-3-(propane-1-sulfonylamino)-phenyl]amide (0.0528 g, 0.123 mmol, 44.0% yield) as a yellow solid. ¹H NMR (400 MHz, d₆-DMSO) δ 13.86 (s, 1H), 10.13 (s, 1H), 8.63-8.65 (dd, 1H), 8.55 (s, 1H), 8.51-8.54 (dd, 1H), 8.39 (br s, 1H), 8.26 (br s, 1H), 7.65-7.72 (m, 2H), 7.40-7.43 (dd, 1H), 3.13-3.17 (m, 2H), 1.73-1.82 (m, 2H), 0.97 (t, 3H). m/z 429.2 (LC/MS positive ionization) [M+1].

### Example 29

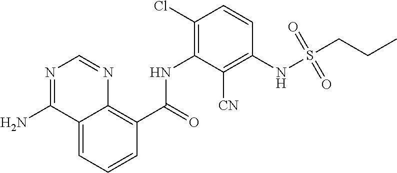 [SMILES: CCCS(=O)(=O)Nc1ccc(Cl)c(NC(=O)c2cccc3c(N)ncnc23)c1C#N]

### 4-Amino-quinazoline-8-carboxylic acid [6-chloro-2-cyano-3-(propane-1-sulfonylamino)-phenyl]-amide

To N-(3-amino-4-chloro-2-cyanophenyl)propane-1-sulfonamide (0.2879 g, 1.052 mmol) was added a solution of 4-chloroquinazoline-8-carbonyl chloride (0.2388 g, 1.052 mmol) (Example 1, Step B) in 6 mL chloroform. The mixture was stirred at 55° C. in a sealed vial overnight. The reaction mixture was evaporated, the resulting orange residue was suspended in 4 mL isopropanol and, to this, was added 7 M ammonia in methanol (1.503 mL, 10.52 mmol). The mixture was stirred at 40° C. in a sealed vial. After 2.5 hours, the reaction mixture was evaporated, partitioned between water (adjusted to pH 4 with 10% citric acid) and EtOAc. The emulsion was filtered and the layers separated. The organic layer was washed with brine, dried over MgSO₄, filtered, and evaporated to yield 0.43 g yellow-orange solid. The material was absorbed on silica gel and chromatographed on a silica gel column, eluting with EtOAc, to afford 4-amino-quinazoline-8-carboxylic acid [6-chloro-2-cyano-3-(propane-1-sulfonylamino)-phenyl]amide (0.133 g, 0.2989 mmol, 28.42% yield) as a yellow solid. ¹H NMR (400 MHz, d₆-DMSO) δ 14.05 (s, 1H), 10.32 (br s, 1H), 8.67-8.70 (dd, 1H), 8.59 (s, 1H), 8.54-8.57 (dd, 1H), 8.43 (br s, 1H), 8.28 (br s, 1H), 7.92 (d, 1H), 7.71 (t, 1H), 7.46 (d, 1H), 3.19-3.24 (m, 2H), 1.75-1.84 (m, 2H), 1.00 (t, 3H). m/z 445.2 (LC/MS positive ionization) [M+1].

### Example 30

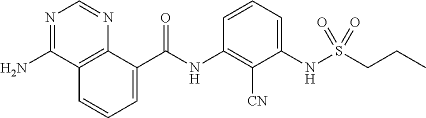 [SMILES: CCCS(=O)(=O)Nc1cccc(NC(=O)c2cccc3c(N)ncnc23)c1C#N]

### 4-Amino-quinazoline-8-carboxylic acid [2-cyano-3-(propane-1-sulfonylamino)-phenyl]-amide

To N-(3-amino-2-cyanophenyl)propane-1-sulfonamide (0.34 g, 1.4 mmol) in 10 mL chloroform in a sealed vial was added 4-chloroquinazoline-8-carbonyl chloride (0.42 g, 1.8 mmol) (Example 1, Step B) and the mixture heated at 55° C. overnight. The reaction mixture was evaporated, taken up in 7 mL dioxane and a gentle stream of ammonia gas passed in for about 3 minutes. The reaction vial was sealed and stirred at 40° C. After 2 hours, the reaction mixture was evaporated, the residue taken up in 0.5 M NaOH and washed with 2 portions EtOAc. The aqueous layer was neutralized to pH 4 with concentrated HCl and the suspension extracted with EtOAc containing about 10% isopropanol. The organic layer was washed with brine, dried over MgSO₄, filtered, and evaporated to yield 0.29 g orange solid. This material was absorbed on silica gel and chromatographed on a silica gel plug, eluting with EtOAc, to afford 4-Amino-quinazoline-8-carboxylic acid [2-cyano-3-(propane-1-sulfonylamino)-phenyl]-amide (0.064 g, 0.16 mmol, 11% yield) as a pale yellow solid. ¹H NMR (400 MHz, d₆-DMSO) δ 10.13 (s, 1H), 8.72 (d, 1H), 8.58 (d, 1H), 8.53-8.56 (m, 1H), 8.43 (br s, 1H), 8.30 (br s, 1H), 7.69-7.74 (m, 2H), 7.24 (d, 1H), 3.20 (t, 2H), 1.79-1.86 (m, 2H), 1.01 (t, 3H). m/z 411.2 (LC/MS positive ionization) [M+1].

### Example 31

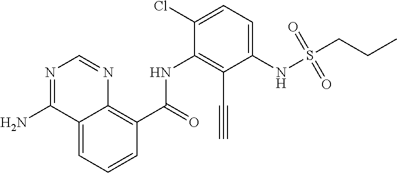 [SMILES: C#Cc1c(NS(=O)(=O)CCC)ccc(Cl)c1NC(=O)c1cccc2c(N)ncnc12]

### 4-Amino-quinazoline-8-carboxylic acid [6-chloro-2-ethynyl-3-(propane-1-sulfonylamino)-phenyl]-amide

Step A:

4-Amino-N-(6-chloro-3-(propylsulfonamido)-2-((triisopropylsilyl)ethynyl)-phenyl)quinazoline-8-carboxamide was prepared according to the general procedure using N-(3-amino-4-chloro-2-((triisopropylsilyl)ethynyl)phenyl)propane-1-sulfonamide and was used directly in the deprotection without further purification.

Step B:

4-Amino-N-(6-chloro-3-(propylsulfonamido)-2-((triisopropylsilyl)ethynyl)-phenyl)quinazoline-8-carboxamide (0.021 g, 0.035 mmol) was dissolved in THF (1 mL). TBAF (0.035 mL, 0.035 mmol) was added, followed by stirring at room temperature for 1 hour. The mixture was partitioned between EtOAc and water. The organic layer was washed with brine, dried over Na₂SO₄ and concentrated. The product was purified by Biotage chromatography eluting with hexanes/EtOAc to give 4-amino-quinazoline-8-carboxylic acid [6-chloro-2-ethynyl-3-(propane-1-sulfonylamino)-phenyl]-amide (4.5 mg, 29% for three steps) as a solid. ¹H NMR (400 MHz, CDCl₃) δ 13.60. (s, 1H), 8.96-8.98 (m, 1H), 8.71 (s, 1H), 7.97-7.99 (m, 1H), 7.66-7.70 (t, 1H), 7.56-7.58 (d, 1H), 7.48-7.50 (d, 1H), 7.10 (br s, 1H), 5.85 (br s, 2H), 3.66 (s, 1H), 3.10-3.14 (m, 2H), 1.84-1.93 (m, 2H), 1.03-1.07 (t, 3 h). m/z (APCI-neg) M−1=442.1, 444.0.

### Example 32

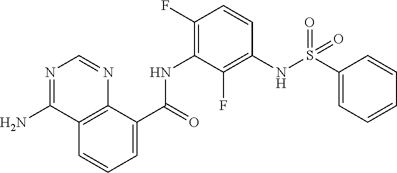 [SMILES: Nc1ncnc2c(C(=O)Nc3c(F)ccc(NS(=O)(=O)c4ccccc4)c3F)cccc12]

### 4-Amino-quinazoline-8-carboxylic acid (3-benzenesulfonylamino-2,6-difluoro-phenyl)-amide

N-(3-Amino-2,4-difluorophenyl)benzenesulfonamide (279.0 mg, 0.981 mmol) was dissolved in CHCl₃ (6.5 mL) and treated with 4-chloroquinazoline-8-carbonyl chloride (222.8 mg, 0.981 mmol). The reaction mixture was heated to 60° C., stirred for 16 hours, and then cooled to ambient temperature and concentrated. The crude reaction mixture was dissolved in 8 mL 1,4-dioxane and anhydrous ammonia gas was passed through the solution for 5 minutes. The vial was sealed, heated to 100° C. for 5 hours, and then cooled to ambient temperature and concentrated. Purification via flash chromatography eluting with a gradient of 10->80% acetone:hexanes afforded 4-amino-quinazoline-8-carboxylic acid (3-benzenesulfonylamino-2,6-difluoro-phenyl)-amide (111.8 mg, 0.246 mmol, 25.0% yield). ¹H NMR (400 MHz, DMSO-d₆)=13.179 (s, 1H), 10.256 (s, 1H), 8.617-8.594 (m, 1H), 8.539 (s, 1H), 8.527-8.502 (m, 1H), 8.367-8.192 (m, 2H), 7.758-7.741 (d, 2H), 7.684-7.642 (t, 2H), 7.602-7.565 (t, 2H), 7.193-7.109 (m, 2H). LC/MS: m/z 456.1 [M+1].

### Example 33

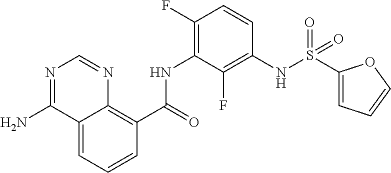 [SMILES: Nc1ncnc2c(C(=O)Nc3c(F)ccc(NS(=O)(=O)c4ccco4)c3F)cccc12]

### 4-Amino-quinazoline-8-carboxylic acid [2,6-difluoro-3-(furan-2-sulfonylamino)-phenyl]-amide

N-(3-Amino-2,4-difluorophenyl)furan-2-sulfonamide (47.8 mg, 0.174 mmol) was dissolved in CHCl₃ (1.7 mL) and treated with 4-chloroquinazoline-8-carbonyl chloride (39.6 mg, 0.174 mmol). The reaction mixture was heated to 60° C., stirred for 16 hours, and then cooled to ambient temperature and concentrated. The crude reaction mixture was dissolved in 8 mL 1,4-dioxane (3 mL) and anhydrous ammonia gas was passed through the solution for 5 minutes. The vial was sealed, heated to 100° C. for 5 hours, and then cooled to ambient temperature and concentrated. Purification via flash chromatography eluting with a gradient of 10->80% acetone:hexanes afforded 4-amino-quinazoline-8-carboxylic acid [2,6-difluoro-3-(furan-2-sulfonylamino)-phenyl]-amide (24.9 mg, 0.056 mmol, 32.1% yield). ¹H NMR (400 MHz, DMSO-d₆) δ=13.249 (s, 1H), 10.565 (s, 1H), 8.637-8.615 (d, 1H), 8.564 (s, 1H), 8.535-8.512 (d, 1H), 8.413-8.191 (m, 2H), 8.015 (m, 1H), 7.694-7.655 (t, 1H), 7.232-7.130 (m, 2H), 7.076-7.067 (d, 1H), 6.676-6.662 (m, 1H). LC/MS: m/z 446.1 [M+1].

### Example 34

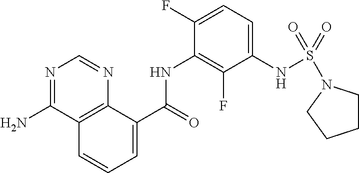 [SMILES: Nc1ncnc2c(C(=O)Nc3c(F)ccc(NS(=O)(=O)N4CCCC4)c3F)cccc12]

### 4-Amino-quinazoline-8-carboxylic acid [2,6-difluoro-3-(pyrrolidine-1-sulfonylamino)-phenyl]-amide

Step A:

To N-(3-amino-2,4-difluorophenyl)propane-1-sulfonamide (0.250 g, 0.999 mmol) in DMF (4.5 mL) was added potassium carbonate (0.414 g, 3.00 mmol) and pyrrolidine-1-sulfonyl chloride (0.196 mL, 1.50 mmol). The suspension was stirred at ambient temperature for 18 hours. To the suspension was then added 1 mL of 2M NaOH which stirred at ambient temperature for 1 hour. The resulting solution was diluted with water (20 mL) and brought to pH 9 with HCl followed by extraction with EtOAc (3×15 mL). The concentrated organics were purified via silica gel chromatography eluting with 1:1 Hexane-EtOAc to provide N-(3-amino-2,4-difluorophenyl)pyrrolidine-1-sulfonamide (184 mg, 66%).

Step B:

To 4-(2,4-dimethoxybenzylamino)quinazoline-8-carboxylic acid (0.0529 g, 0.156 mmol), in DMF (1.0 mL) was added N-(3-amino-2,4-difluorophenyl)pyrrolidine-1-sulfonamide (0.036 g, 0.130 mmol), 2-(3H-[1,2,3]triazolo[4,5-b]pyridin-3-yl)-1,1,3,3-tetramethylisouronium hexafluorophosphate(V) (0.0592 g, 0.156 mmol), and N-ethyl-N-isopropylpropan-2-amine (0.0503 g, 0.389 mmol) which was stirred at ambient temperature for 18 hours. The solution was diluted with EtOAc (15 mL) then washed with a 1:1:1 brine-bicarbonate-water mixture (3×10 mL). The concentrated organics were purified via silica gel chromatography eluting with 6:4 EtOAc/hexanes to provide N-(2,6-difluoro-3-(pyrrolidine-1-sulfonamido)phenyl)-4-(2,4-dimethoxybenzylamino)quinazoline-8-carboxamide (0.031 g, 0.0525 mmol, 40%).

Step C:

N-(2,6-Difluoro-3-(pyrrolidine-1-sulfonamido)phenyl)-4-(2,4-dimethoxybenzyl-amino)quinazoline-8-carboxamide (0.031 g, 0.052 mmol) was dissolved in TFA (0.6 mL) and then warmed to 85° C. for 1 hour. The solution was cooled, concentrated, and then partitioned between EtOAc and sodium bicarbonate (aq). The organic layer was dried over Na₂SO₄, concentrated, then purified via trituration with DCM to provide 4-amino-quinazoline-8-carboxylic acid [2,6-difluoro-3-(pyrrolidine-1-sulfonylamino)-phenyl]-amide (0.021 g, 0.047 mmol, 90%). ¹H NMR (400 MHz, MeOH-d₄) δ 8.69-8.78 (m, 1H), 8.55 (s, 1H), 8.33-8.42 (m, 1H), 7.59-7.70 (m, 1H), 7.44-7.56 (m, 1H), 6.98-7.10 (m, 1H), 3.33-3.36 (m, 4H), 1.80-1.90 (m, 4H); m/z (APCI-pos) M+1=449.1.

Examples 35-39 listed in Table 2 were prepared using similar procedures as described in Example 34 using appropriate starting materials.

TABLE 2

| Example | | | MS m/z | ¹H NMR δ |
|---|---|---|---|---|
| no. | Structure | Name | [M + H]⁺ | (400 MHz, CD₃OD) |
| 35 | 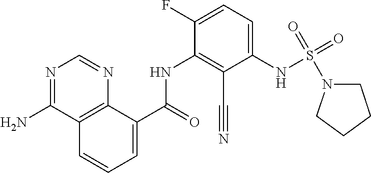 [SMILES: N#Cc1c(NS(=O)(=O)N2CCCC2)ccc(F)c1NC(=O)c1cccc2c(N)ncnc12] | 4-Amino-quinazoline-8- carboxylic acid [2-cyano- 6-fluoro-3-(pyrrolidine-1- sulfonylamino)-phenyl]- amide | 456.2 | 8.76-8.79 (m, 1H), 8.57 (s, 1H), 8.40-8.44 (m, 1H), 7.66- 7.71 (m, 1H), 7.61-7.65 (m, 1H), 7.51-7.57 (m, 1H), 3.32- 3.37 (m, 4H), 1.90-1.94 (m, 4H) |
| 36 | 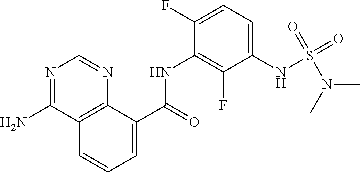 [SMILES: CN(C)S(=O)(=O)Nc1ccc(F)c(NC(=O)c2cccc3c(N)ncnc23)c1F] | 4-Amino-N-(3-(N,N- dimethylsulfamoylamino)- 2,6- difluorophenyl)quinazoline- 8-carboxamide | 423.2 | 8.74-8.77 (m, 1H), 8.56 (s, 1H), 8.39-8.43 (m, 1H), 7.65- 7.70 (m, 1H), 7.48-7.55 (m, 1H), 7.06-7.12 (m, 1H), 2.81 (s, 6H) |
| 37 | 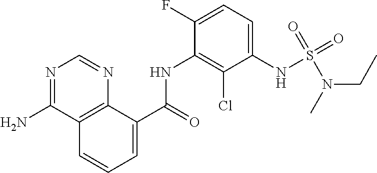 [SMILES: CCN(C)S(=O)(=O)Nc1ccc(F)c(NC(=O)c2cccc3c(N)ncnc23)c1Cl] | 4-Amino-N-(2-chloro-3- (N-ethyl-N- methylsulfamoylamino)- 6- fluorophenyl)quinazoline- 8-carboxamide | 453.1 | 8.74-8.77 (m, 1H), 8.56 (s, 1H), 8.39-8.43 (m, 1H), 7.65- 7.70 (m, 1H), 7.59-7.64 (m, 1H), 7.20-7.26 (m, 1H), 3.18- 3.25 (m, 2H), 2.83 (s, 3H), 1.08-1.13 (m, 3H) |
| 38 | 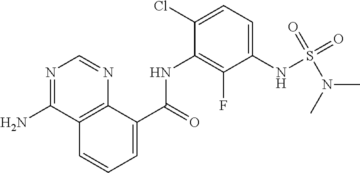 [SMILES: CN(C)S(=O)(=O)Nc1ccc(Cl)c(NC(=O)c2cccc3c(N)ncnc23)c1F] | 4-Amino-N-(6-chloro-3- (N,N- dimethylsulfamoylamino)- 2- fluorophenyl)quinazoline- 8-carboxamide | 439.1 | 8.74-8.77 (m, 1H), 8.56 (s, 1H), 8.40-8.44 (m, 1H), 7.65- 7.70 (m, 1H), 7.50-7.56 (m, 1H), 7.32-7.36 (m, 1H), 2.82 (s, 6H) |
| 39 | 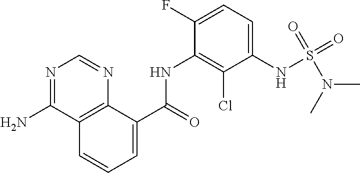 [SMILES: CN(C)S(=O)(=O)Nc1ccc(F)c(NC(=O)c2cccc3c(N)ncnc23)c1Cl] | 4-Amino-N-(2-chloro-3- (N,N- dimethylsulfamoylamino)- 6- fluorophenyl)quinazoline- 8-carboxamide | 439.1 | 8.73-8.77 (m, 1H), 8.56 (s, 1H), 8.38-8.42 (m, 1H), 7.65- 7.69 (m, 1H), 7.61-7.65 (m, 1H), 7.20-7.25 (m, 1H), 2.82 (s, 6H) |

### Example 40

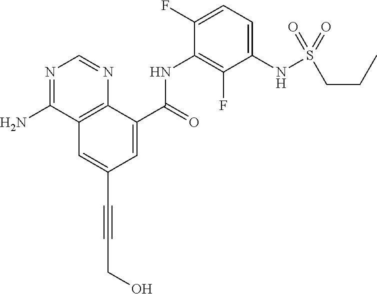 [SMILES: CCCS(=O)(=O)Nc1ccc(F)c(NC(=O)c2cc(C#CCO)cc3c(N)ncnc23)c1F]

### 4-Amino-6-(3-hydroxy-prop-1-ynyl)-quinazoline-8-carboxylic acid [2,6-difluoro-3-(propane-1-sulfonylamino)-phenyl]-amide

Step A:

To 2-amino-5-iodoisophthalic acid (0.160 g, 0.521 mmol) was added formamidine acetate (0.163 g, 1.56 mmol) and formamide (0.0213 mL, 0.521 mmol). The solid mixture was warmed to 170° C. for 10 min, then 185° C. for 5 minutes then cooled and diluted with water (5 mL). The solids were filtered and dried under vacuum to afford 4-hydroxy-6-iodoquinazoline-8-carboxylic acid (0.130 mg, 79%).

Step B:

To 4-hydroxy-6-iodoquinazoline-8-carboxylic acid (1.5 g, 4.7 mmol) in EtOH (24 mL) was added H₂SO₄ (0.053 mL, 0.95 mmol) which was stirred at reflux for 16 hours. The suspension was diluted with EtOH (20 mL) and H₂SO₄ (0.053 mL, 0.95 mmol). Stirring was continued at reflux for 16 hours. The resulting solution was cooled and concentrated under reduced pressure to afford ethyl 4-hydroxy-6-iodoquinazoline-8-carboxylate (1.6 g, 98%).

Step C:

To ethyl 4-hydroxy-6-iodoquinazoline-8-carboxylate (1.6 g, 4.65 mmol) suspended in CH₃CN (23 mL) was added benzotriazol-1-yl-oxytripyrrolidinophosphonium hexafluorophosphate (“PyBOP”) (3.15 g, 6.04 mmol), and 2,3,4,6,7,8,9,10-octahydropyrimido[1,2-a]azepine (1.77 mL, 11.6 mmol). After 30 min, (2,4-dimethoxyphenyl)methanamine (1.41 mL, 9.30 mmol) was added and the solution was stirred at ambient temperature for 48 hours. The solution was concentrated under reduce pressure and purified via silica gel chromatography eluting with 1:1 Hexane-EtOAc to provide ethyl 4-(2,4-dimethoxybenzylamino)-6-iodoquinazoline-8-carboxylate (1.0 g, 43%).

Step D:

To ethyl 4-(2,4-dimethoxybenzylamino)-6-iodoquinazoline-8-carboxylate (0.055 mg, 0.111 mmol) in THF (0.7 mL) was added tert-butyldimethyl(prop-2-ynyloxy)silane (0.0384 mL, 0.223 mmol), TEA (0.155 mL, 1.11 mmol), Cu(I)I (2.1 mg, 0.0111 mmol), and PdCl₂(PPh₃)₂ (7.8 mg, 0.0111 mmol). The suspension was stirred under Argon at ambient temperature for 16 hours then concentrated under reduced pressure. The residue was purified via silica gel chromatography eluting with 1:1 hexane-EtOAc to afford ethyl 6-(3-(tert-butyldimethylsilyloxy)prop-1-ynyl)-4-(2,4-dimethoxybenzylamino)quinazoline-8-carboxylate (0.044 mg, 73%).

Step E:

To ethyl 6-(3-(tert-butyldimethylsilyloxy)prop-1-ynyl)-4-(2,4-dimethoxy-benzylamino)quinazoline-8-carboxylate (0.044 mg, 0.082 mmol) in THF (0.4 mL) and water (0.1 mL) was added LiOH—H₂O (0.017 g, 0.41 mmol) which was stirred at 65° C. for 4 hours. The solution was cooled, diluted with water, and the pH was adjusted to 5. Extraction (3×10 mL) with EtOAc and concentration under reduced pressure afforded 4-(2,4-dimethoxybenzylamino)-6-(3-hydroxyprop-1-ynyl)quinazoline-8-carboxylic acid (0.014 g, 46%).

Step F:

To 4-(2,4-dimethoxybenzylamino)-6-(3-hydroxyprop-1-ynyl)quinazoline-8-carboxylic acid (0.017 g, 0.043 mmol) in DMF (0.5 mL) was added Hunig's base (0.017 g, 0.13 mmol), HATU (0.020 g, 0.052 mmol), and N-(3-amino-2,4-difluorophenyl)propane-1-sulfonamide (0.013 g, 0.052 mmol). The solution was stirred at ambient temperature for 16 h followed by dilution with EtOAc (15 mL) which was washed with water and brine. The concentrated organics were purified via silica gel chromatography eluting with 3:7 Hexane-EtOAc to provide N-(2,6-difluoro-3-(propylsulfonamido)phenyl)-4-(2,4-dimethoxybenzyl-amino)-6-(3-hydroxyprop-1-ynyl)quinazoline-8-carboxamide (0.006 g, 22%).

Step G:

N-(2,6-Difluoro-3-(propylsulfonamido)phenyl)-4-(2,4-dimethoxybenzyl-amino)-6-(3-hydroxyprop-1-ynyl)quinazoline-8-carboxamide (0.0023 g, 0.0037 mmol) was dissolved in TFA (0.2 mL) and warmed to 85° C. for 1 hour. The cooled solution was concentrated then partitioned between EtOAc and sodium bicarbonate (aq). The organics were concentrated and the residue was triturated with DCM to provide 4-amino-6-(3-hydroxy-prop-1-ynyl)-quinazoline-8-carboxylic acid [2,6-difluoro-3-(propane-1-sulfonyl-amino)phenyl]amide (0.0010 g, 0.0021 mmol, 57%). ¹H NMR (400 MHz, MeOH-d₄) δ 8.70-8.72 (m, 1H), 8.55 (s, 1H), 8.50-8.52 (m, 1H), 7.37-7.44 (m, 1H), 6.94-7.01 (m, 1H), 4.45 (2, 2H), 2.98-3.03 (m, 2H), 1.80-1.90 (m, 2H), 1.00-1.06 (m, 3H); m/z (APCI-pos) M+1=476.0.

### Example 41

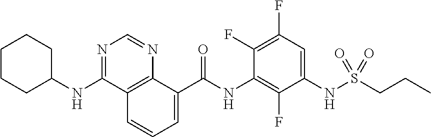 [SMILES: CCCS(=O)(=O)Nc1cc(F)c(F)c(NC(=O)c2cccc3c(NC4CCCCC4)ncnc23)c1F]

### 4-Cyclohexylamino-quinazoline-8-carboxylic acid [2,3,6-trifluoro-5-(propane-1-sulfonylamino)-phenyl]-amide

4-Chloro-N-(2,3,6-trifluoro-5-(propylsulfonamido)phenyl)quinazoline-8-carboximide (31.6 mg, 0.07 mmol), aminocyclohexane (79 ul, 0.7 mmol), and N,N-diisopropylethylamine (120 ul, 0.7 mmol) were combined in isopropanol (1 mL), and the mixture was stirred at 70° C. for 1 hour. The solvent was removed under reduced pressure and the crude product purified by prep HPCL to obtain 4-cyclohexylamino-quinazoline-8-carboxylic acid [2,3,6-trifluoro-5-(propane-1-sulfonylamino)-phenyl]-amide (10.2 mg, 28.3%). ¹H NMR (400 MHz, DMSO-d₆) δ 13.22 (s, 1H), 8.64 (d, J=5.0 Hz, 3H), 8.41 (d, J=7.5 Hz, 1H), 8.28 (s, 1H), 7.68 (t, J=7.7 Hz, 1H), 7.27 (d, J=13.5 Hz, 1H), 6.65 (s, 2H), 4.27 (s, 2H), 2.81 (d, J=6.5 Hz, 3H), 1.98 (d, J=12.4 Hz, 2H), 1.83-1.71 (m, 2H), 1.74-1.56 (m, 3H), 1.43 (s, 4H), 1.19 (d, J=12.6 Hz, 1H), 0.93 (t, J=7.5 Hz, 3H). LC-MS [M+1] m/z 522.1.

Examples 42-52 listed in Table 3 were prepared applying the procedure described in Example 41 and using appropriate amino building blocks.

TABLE 3

| Example | | | MS m/z | ¹H NMR δ |
|---|---|---|---|---|
| no. | Structure | Name | [M + H]⁺ | (400 MHz, DMSO-d6) |
| 42 | 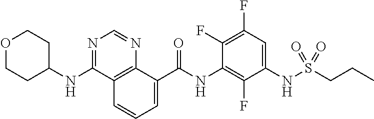 [SMILES: CCCS(=O)(=O)Nc1cc(F)c(F)c(NC(=O)c2cccc3c(NC4CCOCC4)ncnc23)c1F] | 4-(Tetrahydropyran-4- ylamino)-quinazoline- 8-carboxylic acid [2,3,6-trifluoro-5- (propane-1- sulfonylamino)- phenyl]-amide | 524.1 | 13.40 (s, 1H), 10.13 (s, 1H), 8.66 (s, 3H), 8.51 (d, J = 7.4 Hz, 1H), 7.72 (t, J = 7.6 Hz, 1H), 7.42 (dd, J = 19.5, 7.7 Hz, 1H), 6.52 (s, 1H), 4.49 (s, 1H), 3.95 (d, J = 9.6 Hz, 2H), 3.44 (dd, J = 28.9, 17.2 Hz, 3H), 3.16-2.97 (m, 2H), 1.92 (d, J = 12.0 Hz, 2H), 1.73 (dd, J = 14.8, 8.3 Hz, 4H), 0.97 (s, 3H). |
| 43 | 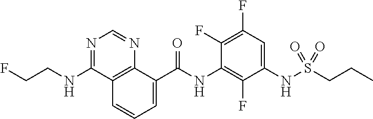 [SMILES: CCCS(=O)(=O)Nc1cc(F)c(F)c(NC(=O)c2cccc3c(NCCF)ncnc23)c1F] | 4-(2-Fluoro- ethylamino)- quinazoline-8- carboxylic acid [2,3,6- trifluoro-5-(propane-1- sulfonylamino)- phenyl]-amide | 486.1 | 13.29 (s, 1H), 9.01 (s, 1H), 8.63 (dd, J = 25.7, 9.5 Hz, 3H), 8.17 (s, 0H), 7.74 (s, 1H), 7.38 (dd, J = 19.8, 7.7 Hz, 1H), 4.70 (d, J = 47.4 Hz, 2H), 3.92 (dd, J = 26.6, 4.9 Hz, 2H), 3.10-2.88 (m, 2H), 1.72 (d, J = 7.3 Hz, 2H), 0.96 (t, J = 7.5 Hz, 3H) |
| 44 | 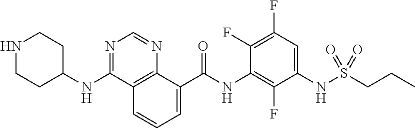 [SMILES: CCCS(=O)(=O)Nc1cc(F)c(F)c(NC(=O)c2cccc3c(NC4CCNCC4)ncnc23)c1F] | 4-(Piperidin-4- ylamino)-quinazoline- 8-carboxylic acid [2,3,6-trifluoro-5- (propane-1- sulfonylamino)- phenyl]-amide | 523.1 | 12.97 (s, 1H), 8.76 (s, 1H), 8.66 (d, J = 7.6 Hz, 1H), 8.23 (d, J = 9.0 Hz, 2H), 7.70 (s, 1H), 7.36-7.22 (m, 1H), 4.42 (d, J = 13.1 Hz, 2H), 2.95-2.71 (m, 3H), 2.01 (d, J = 11.6 Hz, 3H), 1.65 (dd, J = 15.0, 8.7 Hz, 4H), 0.94 (t, J = 7.4 Hz, 3H) |
| 45 | 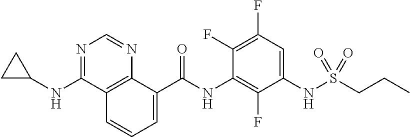 [SMILES: CCCS(=O)(=O)Nc1cc(F)c(F)c(NC(=O)c2cccc3c(NC4CC4)ncnc23)c1F] | 4-Cyclopropylamino- quinazoline-8- carboxylic acid [2,3,6- trifluoro-5-(propane-1- sulfonylamino)- phenyl]-amide | 480.1 | 13.44 (s, 1H), 9.96 (s, 1H), 8.72 (s, 2H), 8.64 (d, J = 7.4 Hz, 1H), 8.56 (d, J = 8.2 Hz, 1H), 7.70 (t, J = 7.7 Hz, 1H), 7.45 (dd, J = 18.2, 8.1 Hz, 1H), 3.20-3.07 (m, 3H), 1.75 (dd, J = 15.0, 7.3 Hz, 2H), 0.98 (s, 3H), 0.86 (d, J = 6.8 Hz, 2H), 0.73 (s, 2H) |
| 46 | 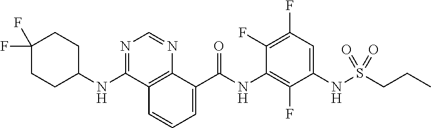 [SMILES: CCCS(=O)(=O)Nc1cc(F)c(F)c(NC(=O)c2cccc3c(NC4CCC(F)(F)CC4)ncnc23)c1F] | 4-(4,4-Difluoro- cyclohexylamino)- quinazoline-8- carboxylic acid [2,3,6- trifluoro-5-(propane-1- sulfonylamino)- phenyl]-amide | 558.1 | 13.37 (s, 1H), 10.74-9.44 (m, 1H), 8.81-8.56 (m, 3H), 8.48 (d, J = 7.5 Hz, 1H), 8.15 (s, 0H), 7.72 (t, J = 7.9 Hz, 1H), 7.42 (dd, J = 19.3, 7.8 Hz, 1H), 6.52 (s, 0H), 4.49 (s, 1H), 3.19-2.95 (m, 2H), 2.05 (d, J = 17.5 Hz, 7H), 1.86-1.59 (m, 4H), 0.97 (t, J = 7.3 Hz, 3H) |
| 47 | 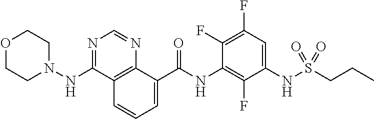 [SMILES: CCCS(=O)(=O)Nc1cc(F)c(F)c(NC(=O)c2cccc3c(NN4CCOCC4)ncnc23)c1F] | 4-(Morpholin-4- ylamino)-quinazoline- 8-carboxylic acid [2,3,6-trifluoro-5- (propane-1- sulfonylamino)- phenyl]-amide | 525.1 | 12.97 (s, 1H), 8.76 (s, 1H), 8.66 (d, J = 7.6 Hz, 1H), 8.23 (d, J = 9.0 Hz, 2H), 7.70 (s, 1H), 7.36-7.22 (m, 1H), 4.42 (d, J = 13.1 Hz, 2H), 2.95-2.71 (m, 3H), 2.01 (d, J = 11.6 Hz, 3H), 1.65 (dd, J = 15.0, 8.7 Hz, 4H), 0.94 (t, J = 7.4 Hz, 3H) |
| 48 | 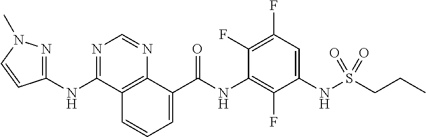 [SMILES: CCCS(=O)(=O)Nc1cc(F)c(F)c(NC(=O)c2cccc3c(Nc4ccn(C)n4)ncnc23)c1F] | 4-(1-Methyl-1H- pyrazol-3-ylamino)-N- (2,3,6-trifluoro-5- (propylsulfonamido) phenyl)quinazoline-8- carboxamide | 520.1 | 13.26 (s, 1H), 10.90 (s, 1H), 9.97 (s, 1H), 8.94 (d, J = 8.5 Hz, 1H), 8.78 (s, 1H), 8.69 (d, J = 7.5 Hz, 1H), 7.77 (t, J = 8.0 Hz, 1H), 7.72 (s, 1H), 7.46 (dd, J = 18.7, 7.7 Hz, 1H), 6.84 (s, 1H), 6.52 (s, 1H), 3.85 (s, 3H), 3.22-3.09 (m, 2H), 1.75 (dd, J = 15.1, 7.2 Hz, 2H), 0.98 (s, 3H) |
| 49 | 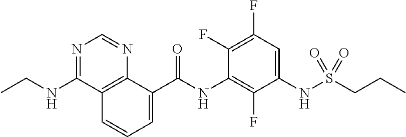 [SMILES: CCCS(=O)(=O)Nc1cc(F)c(F)c(NC(=O)c2cccc3c(NCC)ncnc23)c1F] | 4-Ethylamino- quinazoline-8- carboxylic acid [2,3,6- trifluoro-5-(propane-1- sulfonylamino)- phenyl]-amide | 468.1 | 13.45 (s, 1H), 10.11 (s, 1H), 8.82 (s, 1H), 8.65 (s, 2H), 8.55 (d, J = 8.3 Hz, 1H), 7.70 (t, J = 7.9 Hz, 1H), 7.49- 7.30 (m, 1H), 6.52 (s, 1H), 3.63 (dd, J = 13.1, 6.4 Hz, 2H), 3.10 (d, J = 6.9 Hz, 2H), 1.74 (dd, J = 14.9, 7.3 Hz, 2H), 1.26 (t, J = 7.1 Hz, 3H), 0.99 (d, J = 7.3 Hz, 3H) |
| 50 | 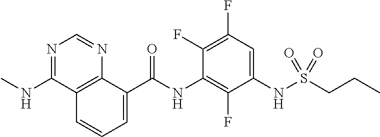 [SMILES: CCCS(=O)(=O)Nc1cc(F)c(F)c(NC(=O)c2cccc3c(NC)ncnc23)c1F] | 4-Methylamino- quinazoline-8- carboxylic acid [2,3,6- trifluoro-5-(propane-1- sulfonylamino)- phenyl]-amide | 454.1 | 13.43 (s, 1H), 11.13-9.62 (m, 2H), 8.86 (d, J = 3.8 Hz, 1H), 8.67 (s, 2H), 8.50 (d, J = 8.2 Hz, 1H), 8.39 (s, 0H), 8.15 (s, 0H), 7.71 (s, 1H), 7.42 (dd, J = 19.2, 7.9 Hz, 1H), 6.53 (s, 1H), 3.08 (d, J = 4.1 Hz, 5H), 1.73 (d, J = 7.4 Hz, 3H), 0.97 (t, J = 7.4 Hz, 4H) |
| 51 | 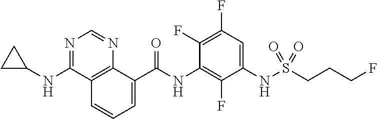 [SMILES: O=C(Nc1c(F)c(F)cc(NS(=O)(=O)CCCF)c1F)c1cccc2c(NC3CC3)ncnc12] | 4-(Cyclopropylamino)- N-(2,3,6-trifluoro-5-(3- fluoropropylsulfonamido) phenyl)quinazoline- 8-carboxamide | 498.1 | 13.46 (s, 1H), 10.09 (s, 1H), 8.72 (s, 2H), 8.64 (d, J = 7.4 Hz, 1H), 8.56 (d, J = 8.4 Hz, 1H), 7.69 (dd, J = 19.1, 11.6 Hz, 1H), 7.47 (dd, J = 18.0, 7.8 Hz, 1H), 4.61 (t, J = 5.8 Hz, 1H), 4.49 (t, J = 5.7 Hz, 1H), 3.27 (s, 2H), 3.12 (s, 1H), 2.12 (dd, J = 17.8, 7.4 Hz, 2H), 0.86 (d, J = 6.6 Hz, 2H), 0.73 (s, 2H) |
| 52 | 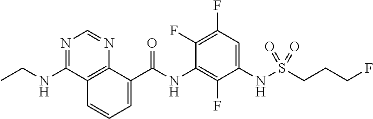 [SMILES: CCNc1ncnc2c(C(=O)Nc3c(F)c(F)cc(NS(=O)(=O)CCCF)c3F)cccc12] | 4-Ethylamino- quinazoline-8- carboxylic acid [2,3,6- trifluoro-5-(3-fluoro- propane-1- sulfonylamino)- phenyl]-amide | 486.1 | 13.44 (s, 1H), 10.36-9.81 (m, 1H), 8.82 (s, 1H), 8.65 (s, 2H), 8.55 (d, J = 8.1 Hz, 1H), 7.70 (t, J = 7.8 Hz, 1H), 7.44 (s, 1H), 4.60 (t, J = 5.9 Hz, 1H), 4.48 (t, J = 5.9 Hz, 1H), 3.71-3.48 (m, 2H), 3.20 (s, 2H), 2.10 (d, J = 22.1 Hz, 2H), 1.26 (s, 3H) |

### Example 53

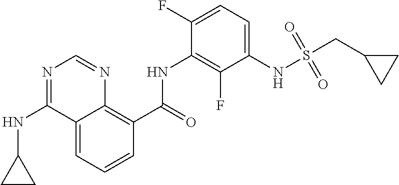 [SMILES: O=C(Nc1c(F)ccc(NS(=O)(=O)CC2CC2)c1F)c1cccc2c(NC3CC3)ncnc12]

### 4-Cyclopropylamino-quinazoline-8-carboxylic acid (3-cyclopropylmethanesulfonylamino-2,6-difluoro-phenyl)-amide

A microwave-vial was charged with 4-chloro-N-(3-(cyclopropyl-methylsulfonamido)-2,6-difluorophenyl)quinazoline-8-carboxamide (35 mg, 0.08 mmol), cyclopropylamine (0.02 ml, 0.23 mmol), and 1,4-dioxane (0.7 ml). The reaction mixture was heated in a microwave reactor at 110° C. for 15 minutes. The reaction mixture was concentrated in vacuo, then purified by reverse phase HPLC to afford 4-(cyclopropylamino)-N-(3-(cyclopropylmethylsulfonamido)-2,6-difluorophenyl)quinazoline-8-carboxamide (10 mg, 27%). ¹H NMR (400 MHz, DMSO) δ 13.24 (s, 1H), 9.75 (s, 1H), 8.75 (d, J=3.6 Hz, 1H), 8.72 (s, 1H), 8.67-8.61 (m, 1H), 8.55 (d, J=7.2 Hz, 1H), 7.70 (t, J=7.9 Hz, 1H), 7.45-7.34 (m, 1H), 7.22 (t, J=8.6 Hz, 1H), 3.16-3.02 (m, 3H), 1.13-1.00 (m, 1H), 0.90-0.81 (m, 2H), 0.76-0.66 (m, 2H), 0.60-0.53 (m, 2H), 0.38-0.32 (m, 2H). m/z (ES-MS) 474.2 [M+1].

### Example 54

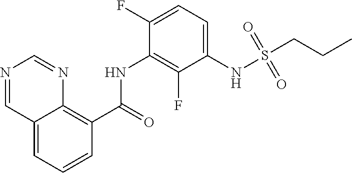 [SMILES: CCCS(=O)(=O)Nc1ccc(F)c(NC(=O)c2cccc3cncnc23)c1F]

### Quinazoline-8-carboxylic acid [2,6-difluoro-3-(propane-1-sulfonylamino)-phenyl]amide

A microwave vessel was charged with 4-chloro-N-(2,6-difluoro-3-(propylsulfonamido)phenyl)quinazoline-8-carboxamide (0.10 g, 0.23 mmol), tri-n-butyl tin hydride (0.12 mL, 0.45 mmol), tetrakis(triphenylphosphine)palladium(0) (0.07 g, 0.06 mmol) and toluene (1.1 mL). The reaction mixture was heated in a microwave reactor at 150° C. for 15 minutes. The palladium was filtered off and the filtrate then concentrated in vacuo. The crude product was purified by reverse phase HPLC to afford quinazoline-8-carboxylic acid [2,6-difluoro-3-(propane-1-sulfonylamino)-phenyl]-amide (0.027 g, 29%). ¹H NMR (400 MHz, DMSO-d₆) δ 12.10 (s, 1H), 9.85 (s, 1H), 9.71 (s, 1H), 9.52 (s, 1H), 8.82 (d, J=6.3 Hz, 1H), 8.50 (d, J=8.1 Hz, 1H), 7.99 (t, J=7.7 Hz, 1H), 7.41 (dd, J=14.5, 8.8 Hz, 1H), 7.25 (t, J=9.0 Hz, 1H), 3.15-3.02 (m, 2H), 1.83-1.70 (m, 2H), 0.99 (t, J=7.4 Hz, 3H). m/z (ES-MS) 407.0 (100%) [M+1].

Examples 55-59 listed in Table 4 were prepared applying the procedure described in Example 54 and using appropriate amino building blocks.

TABLE 4

| Example | | | Ms m/z | ¹H NMR δ |
|---|---|---|---|---|
| no. | Structure | Name | [M + H]⁺ | (400 MHz, DMSO-d₆) |
| 55 | 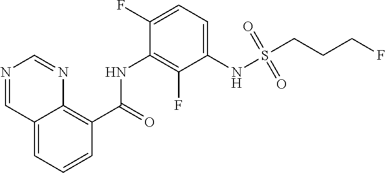 [SMILES: O=C(Nc1c(F)ccc(NS(=O)(=O)CCCF)c1F)c1cccc2cncnc12] | Quinazoline-8- carboxylic acid [2,6- difluoro-3-(3-fluoro- propane-1- sulfonylamino)- phenyl]-amide | 425.2 | 12.11 (s, 1H), 9.97 (s, 1H), 9.86 (s, 1H), 9.52 (s, 1H), 8.82 (d, J = 7.4 Hz, 1H), 8.50 (d, J = 8.0 Hz, 1H), 8.00 (t, J = 7.7 Hz, 1H), 7.40 (dd, J = 14.6, 8.1 Hz, 1H), 7.23 (t, J = 9.4 Hz, 1H), 4.61 (t, J = 5.7 Hz, 1H), 4.49 (t, J = 5.6 Hz, 1H), 3.24-3.13 (m, 2H), 2.21-2.02 (m, 2H) |
| 56 | 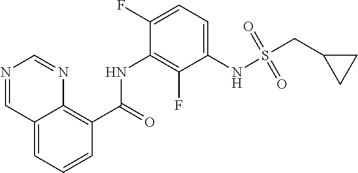 [SMILES: O=C(Nc1c(F)ccc(NS(=O)(=O)CC2CC2)c1F)c1cccc2cncnc12] | Quinazoline-8- carboxylic acid (3- cyclopropylmethane- sulfonylamino- 2,6-difluoro- phenyl)-amide | 419.2 | 12.10 (s, 1H), 9.86 (s, 1H), 9.69 (br s, 1H), 9.52 (s, 1H), 8.82 (d, J = 7.3 Hz, 1H), 8.50 (d, J = 8.1 Hz, 1H), 7.99 (t, J = 7.8 Hz, 1H), 7.43 (dd, J = 14.5, 8.1 Hz, 1H), 7.23 (t, J = 9.1 Hz, 1H), 3.09 (d, J = 7.0 Hz, 2H), 1.13- 1.01 (m, 1H), 0.57 (d, J = 7.5 Hz, 2H), 0.35 (d, J = 4.4 Hz, 2H) |
| 57 | 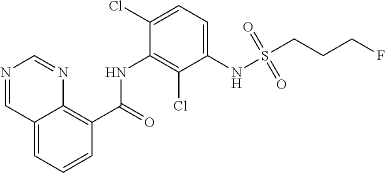 [SMILES: O=C(Nc1c(Cl)ccc(NS(=O)(=O)CCCF)c1Cl)c1cccc2cncnc12] | Quinazoline-8- carboxylic acid [2,6- dichloro-3-(3-fluoro- propane-1- sulfonylamino)- phenyl]-amide | 457.0 | 12.44 (s, 1H), 9.86 (s, 2H), 9.53 (s, 1H), 8.86 (dd, J = 7.4, 1.4 Hz, 1H), 8.50 (dd, J = 8.1, 1.4 Hz, 1H), 8.00 (t, J = 7.7 Hz, 1H), 7.61 (d, J = 8.8 Hz, 1H), 7.51 (d, J = 8.9 Hz, 1H), 4.62 (t, J = 5.9 Hz, 1H), 4.50 (t, J = 6.0 Hz, 1H), 3.31-3.23 (m, 2H), 2.22-2.04 (m, 2H) |
| 58 | 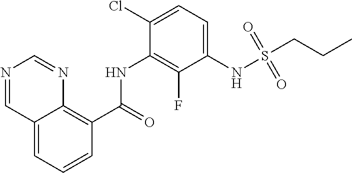 [SMILES: CCCS(=O)(=O)Nc1ccc(Cl)c(NC(=O)c2cccc3cncnc23)c1F] | Quinazoline-8- carboxylic acid [6- chloro-2-fluoro-3- (propane-1- sulfonylamino)- phenyl]-amide | 423.2 | 12.32 (s, 1H), 9.88 (s, 1H), 9.86 (s, 1H), 9.53 (s, 1H), 8.85 (dd, J = 7.4, 1.4 Hz, 1H), 8.51 (dd, J = 8.1, 1.4 Hz, 1H), 8.00 (t, J = 7.7 Hz, 1H), 7.47-7.39 (m, 2H), 3.16-3.07 (m, 2H), 1.81-1.68 (m, 2H), 0.98 (t, J = 7.4 Hz, 3H) |
| 59 | 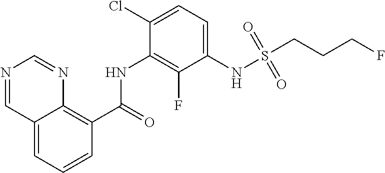 [SMILES: O=C(Nc1c(Cl)ccc(NS(=O)(=O)CCCF)c1F)c1cccc2cncnc12] | Quinazoline-8- carboxylic acid [6- chloro-2-fluoro-3-(3- fluoro-propane-1- sulfonylamino)- phenyl]-amide | 441.2 | 12.33 (s, 1H), 10.02 (s, 1H), 9.86 (s, 1H), 9.52 (s, 1H), 8.85 (dd, J = 7.4, 1.4 Hz, 1H), 8.51 (dd, J = 8.1, 1.4 Hz, 1H), 8.00 (t, J = 7.7 Hz, 1H), 7.48-7.40 (m, 2H), 4.61 (t, J = 6.0 Hz, 1H), 4.49 (t, J = 6.0 Hz, 1H), 3.27-3.16 (m, 2H), 2.21-2.01 (m, 2H) |

### Example 60

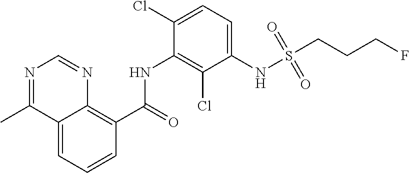 [SMILES: Cc1ncnc2c(C(=O)Nc3c(Cl)ccc(NS(=O)(=O)CCCF)c3Cl)cccc12]

### 4-Methyl-quinazoline-8-carboxylic acid [2,6-dichloro-3-(3-fluoro-propane-1-sulfonylamino)-phenyl]-amide

A microwave-vial was charged with 4-chloro-N-(2,6-dichloro-3-(3-fluoropropylsulfonamido)phenyl)quinazoline-8-carboxamide (0.10 g, 0.20 mmol), trimethylaluminum (0.25 ml, 2M in heptane), tetrakis(triphenylphosphine)palladium(0) (0.024 g, 0.02 mmol) and THF (2 ml). The reaction mixture was heated in a microwave reactor at 75° C. for 15 minutes. The salts were filtered off, and filtrate was concentrated in vacuo, then purified by reverse phase HPLC to afford 4-methyl-quinazoline-8-carboxylic acid [2,6-dichloro-3-(3-fluoro-propane-1-sulfonylamino)-phenyl]-amide (0.011 g, 11%). ¹H NMR (400 MHz, DMSO) δ 12.66 (s, 1H), 9.83 (s, 1H), 9.33 (s, 1H), 8.83 (dd, J=7.4, 1.4 Hz, 1H), 8.64 (dd, J=8.3, 1.4 Hz, 1H), 7.99-7.91 (m, 1H), 7.60 (d, J=8.8 Hz, 1H), 7.50 (d, J=8.9 Hz, 1H), 4.61 (t, J=6.0 Hz, 1H), 4.50 (t, J=6.0 Hz, 1H), 3.04 (s, 3H), 2.22-2.04 (m, 2H). (One peak masked under solvent signal). m/z (ES-MS) 471.0 [M+1].

### Example 61

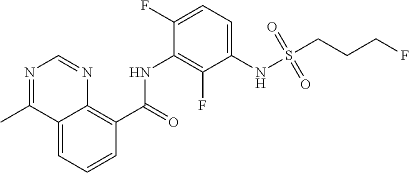 [SMILES: Cc1ncnc2c(C(=O)Nc3c(F)ccc(NS(=O)(=O)CCCF)c3F)cccc12]

### 4-Methyl-quinazoline-8-carboxylic acid [2,6-difluoro-3-(3-fluoro-propane-1-sulfonylamino)-phenyl]-amide

4-Methyl-quinazoline-8-carboxylic acid [2,6-difluoro-3-(3-fluoro-propane-1-sulfonyl-amino)-phenyl]-amide was prepared according to Example 60, substituting 4-chloro-N-(2,6-difluoro-3-(3-fluoropropylsulfonamido)phenyl)quinazoline-8-carboxamide for 4-chloro-N-(2,6-dichloro-3-(3-fluoropropylsulfonamido)phenyl)quinazoline-8-carboxamide. m/z (ES-MS) 439.1 [M+1], R_{T}=4.28 min.

### Example 62

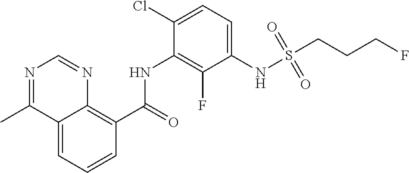 [SMILES: Cc1ncnc2c(C(=O)Nc3c(Cl)ccc(NS(=O)(=O)CCCF)c3F)cccc12]

### 4-Methyl-quinazoline-8-carboxylic acid [6-chloro-2-fluoro-3-(3-fluoro-propane-1-sulfonylamino)-phenyl]-amide

4-Methyl-quinazoline-8-carboxylic acid [6-chloro-2-fluoro-3-(3-fluoro-propane-1-sulfonylamino)-phenyl]-amide was prepared according to Example 60, substituting 4-chloro-N-(6-chloro-2-fluoro-3-(3-fluoropropylsulfonamido)phenyl)quinazoline-8-carboxamide for 4-chloro-N-(2,6-dichloro-3-(3-fluoropropylsulfonamido)phenyl)quinazoline-8-carboxamide. m/z (ES-MS) 455.1 [M+1], R_{T}=4.56 min.

### Example 63

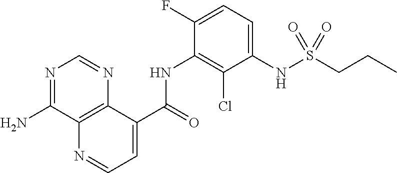 [SMILES: CCCS(=O)(=O)Nc1ccc(F)c(NC(=O)c2ccnc3c(N)ncnc23)c1Cl]

### 4-Amino-pyrido[3,2-d]pyrimidine-8-carboxylic acid [2-chloro-6-fluoro-3-(propane-1-sulfonylamino)-phenyl]-amide

Step A:

A mixture of 2-chloro-3-fluoroisonicotinic acid (2.0 g, 10.0 mmol) in thionyl chloride (42 mL) was heated to 80° C. The homogenous solution was stirred for 2 hours, concentrated under reduced pressure, and pumped dry on high vacuum overnight to give crude 2-chloro-3-fluoroisonicotinoyl chloride. Crude 2-chloro-3-fluoroisonicotinoyl chloride (1.04 g, 5.4 mmol) was added to a stirred solution of N-(3-amino-2-chloro-4-fluorophenyl)-N-(4-methoxybenzyl)propane-1-sulfonamide (2.08 g, 5.4 mmol) in anhydrous CHCl₃ (15 mL), and the reaction mixture was stirred at room temperature overnight. The reaction mixture was quenched by adding sat. aqueous NaHCO₃ solution, followed by extraction with DCM (3×). The combined organic layers were washed with brine, dried over MgSO₄, filtered, and concentrated under reduced pressure. The crude product was purified using flash chromatography (gradient elution, solvent: 0-50% ethyl acetate in heptanes) to afford 2-chloro-N-(2-chloro-6-fluoro-3-(N-(4-methoxybenzyl)propylsulfonamido)phenyl)-3-fluoroisonicotinamide (2.75 g, 94%). ¹H NMR (500 MHz, DMSO-d₆) δ 10.67 (s, 1H), 8.44 (d, 1H), 7.71 (t, 1H), 7.35 (m, 2H), 7.13 (t, 2H), 6.82 (dd, 2H), 4.81 (d, 1H), 4.63 (d, 1H), 3.70 (s, 3H), 3.37-3.20 (m, 2H), 1.80 (d, 2H), 1.01 (dt, 3H). LC/MS: m/z 544.1 [M+1].

Step B:

A 20-mL microwave vial was charged with 2-chloro-N-(2-chloro-6-fluoro-3-(N-(4-methoxybenzyl)propylsulfonamido)phenyl)-3-fluoroisonicotinamide (1.0 g, 1.84 mmol), zinc cyanide (324 mg, 2.76 mmol), 1,1′-bis(diphenylphosphino)ferrocenepalladium (II) chloride (30 mg, 0.0367 mmol), and DMF (10 mL). Nitrogen was passed through the mixture for 15 minutes and the vial was capped. The reaction mixture was subjected to microwave irradiation at 190° C. for 20 min. The reaction mixture was filtered and diluted with water and EtOAc. The organic layer was washed with brine, dried over MgSO₄, filtered, and concentrated under reduced pressure. The crude product was purified using flash chromatography (gradient elution, solvent: 0-50% ethyl acetate in heptanes) to yield N-(2-chloro-6-fluoro-3-(N-(4-methoxybenzyl)propylsulfonamido)phenyl)-2-cyano-3-fluoroisonicotinamide as a light yellow solid (806 mg, 82%). ¹H NMR (500 MHz, CDCl₃) δ 8.77 (d, 1H), 8.27 (s, 1H), 7.94 (s, 1H), 7.14 (d, 2H), 7.04 (m, 2H), 6.80 (d, 2H), 5.06 (d, 1H), 4.44 (d, 1H), 3.78 (s, 3H), 3.13-2.99 (m, 2H), 1.95 (dd, 2H), 1.07 (t, 3H). LC/MS: m/z 535.1 [M+1].

Step C:

To an oven-dried 20-mL microwave vial was added N-(2-chloro-6-fluoro-3-(N-(4-methoxybenzyl)propylsulfonamido)phenyl)-2-cyano-3-fluoroisonicotinamide (500 mg, 0.9 mmol), formamidine acetate (486 mg, 4.67 mmol), and N,N-dimethylacetamide (5 mL, 50 mmol) under a nitrogen atmosphere. The vial was capped and the reaction mixture was subjected to microwave irradiation at 160° C. for 30 min. The reaction mixture was diluted with water and EtOAc. The organic layer was washed with brine, dried over MgSO₄, filtered, and concentrated under reduced pressure. The crude product was purified using flash chromatography (gradient elution, solvent: 70-100% ethyl acetate in heptanes) to yield 4-amino-N-(2-chloro-6-fluoro-3-(N-(4-methoxybenzyl)propylsulfonamido)phenyl)pyrido[3,2-d]pyrimidine-8-carboxamide as a white solid (185 mg, 40%). LC/MS: m/z 533.1 [M+1].

Step D:

To 4-amino-N-(2-chloro-6-fluoro-3-(N-(4-methoxybenzyl)propyl-sulfonamido)phenyl)pyrido[3,2-d]pyrimidine-8-carboxamide (370 mg, 0.66 mmol) in DCM (10 mL) was added trifluoroacetic acid (5 mL). The reaction mixture was stirred at room temperature overnight and then concentrated under reduced pressure. Saturated aqueous NaHCO₃ solution and EtOAc were added, and the organic layer was washed with brine, dried over MgSO₄, filtered, and concentrated under reduced pressure. The crude product was triturated in ether to obtain 4-amino-N-(2-chloro-6-fluoro-3-(propylsulfonamido)phenyl)-pyrido[3,2-d]pyrimidine-8-carboxamide as an off-white solid (200 mg, 70%). ¹H NMR (500 MHz, DMSO-d₆) δ 13.01 (s, 1H), 9.58 (s, 1H), 9.00 (d, 1H), 8.59 (s, 1H), 8.52-8.43 (m, 2H), 8.42 (s, 1H), 7.48 (dd, 1H), 7.39 (t, 1H), 3.19-3.04 (m, 2H), 1.78 (dd, 2H), 0.99 (t, 3H). LC/MS: m/z 439.0 [M+1].

Examples 64-67 listed in Table 5 were prepared applying the procedure described in Example 63 and using appropriate amino building blocks.

TABLE 5

| Example | | | | ¹H NMR δ |
|---|---|---|---|---|
| no. | Structure | Name | MS m/z | (400 MHz, DMSO-d6) |
| 64 | 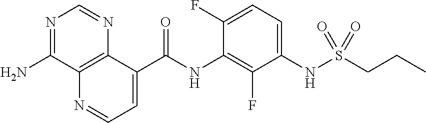 [SMILES: CCCS(=O)(=O)Nc1ccc(F)c(NC(=O)c2ccnc3c(N)ncnc23)c1F] | 4-Amino-pyrido[3,2- d]pyrimidine-8- carboxylic acid [2,6- difluoro-3-(propane-1- sulfonylamino)- phenyl]-amide | 423.1 | 12.77 (s, 1H), 9.00 (s, 1H), 8.58 (s, 1H), 8.48 (m, 1H), 8.46-8.35 (m, 2H), 7.47- 7.29 (m, 1H), 7.21 (t, 1H), 3.13-2.98 (m, 2H), 1.86- 1.65 (m, 2H), 0.98 (t, 3H). |
| 65 | 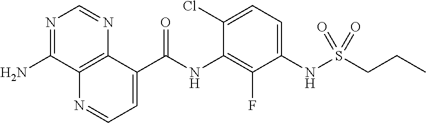 [SMILES: CCCS(=O)(=O)Nc1ccc(Cl)c(NC(=O)c2ccnc3c(N)ncnc23)c1F] | 4-Amino-pyrido[3,2- d]pyrimidine-8- carboxylic acid [6- chloro-2-fluoro-3- (propane-1- sulfonylamino)- phenyl]-amide | 439.0 | 13.02 (s, 1H), 9.94 (s, 1H), 9.00 (d, J = 4.5 Hz, 1H), 8.58 (s, 1H), 8.55 (s, 1H), 8.48 (s, 1H), 8.45 (d, J = 4.5 Hz, 1H), 7.50-7.39 (m, 2H), 3.15- 3.07 (m, 2H), 1.81-1.68 (m, 2H), 0.97 (t, J = 7.4 Hz, 3H). |
| 66 | 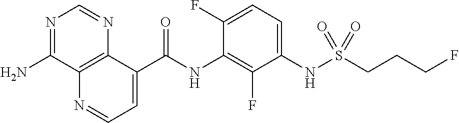 [SMILES: Nc1ncnc2c(C(=O)Nc3c(F)ccc(NS(=O)(=O)CCCF)c3F)ccnc12] | 4-Amino-pyrido[3,2- d]pyrimidine-8- carboxylic acid [2,6- difluoro-3-(3-fluoro- propane-1- sulfonylamino)- phenyl]-amide | 441.0 | 12.81 (s, 1H), 9.85 (s, 1H), 9.00 (d, J = 4.5 Hz, 1H), 8.58 (s, 1H), 8.48 (s, 1H), 8.45- 8.41 (m, 2H), 7.41 (td, J = 8.9, 5.8 Hz, 1H), 7.26 (t, J = 9.2 Hz, 1H), 4.61 (t, J = 6.0 Hz, 1H), 4.49 (t, J = 6.0 Hz, 1H), 3.21 (d, J = 7.8 Hz, 2H), 2.20-2.04 (m, 2H). |
| 67 | 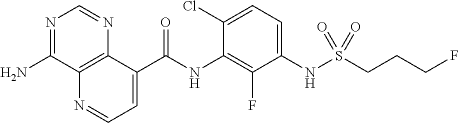 [SMILES: Nc1ncnc2c(C(=O)Nc3c(Cl)ccc(NS(=O)(=O)CCCF)c3F)ccnc12] | 4-Amino-pyrido[3,2- d]pyrimidine-8- carboxylic acid [6- chloro-2-fluoro-3-(3- fluoro-propane-1- sulfonylamino)- phenyl]-amide | 423.1 | 13.02 (s, 1H), 10.05 (s, 1H), 9.00 (d, J = 4.5 Hz, 1H), 8.58 (s, 1H), 8.52 (s, 1H), 8.49- 8.43 (m, 2H), 7.51-7.39 (m, 2H), 4.60 (t, J = 5.9 Hz, 1H), 4.48 (t, J = 6.0 Hz, 1H), 3.25 (dd, J = 8.7, 6.6 Hz, 2H), 2.21- 2.03 (m, 2H). |

### Example 68

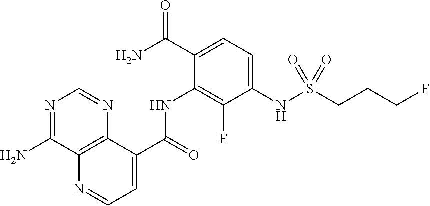 [SMILES: NC(=O)c1ccc(NS(=O)(=O)CCCF)c(F)c1NC(=O)c1ccnc2c(N)ncnc12]

### 4-Amino-pyrido[3,2-d]pyrimidine-8-carboxylic acid [6-carbamoyl-2-fluoro-3-(3-fluoro-propane-1-sulfonylamino)-phenyl]-amide

Step A:

2-Chloro-N-(6-chloro-2-fluoro-3-(3-fluoro-N-(4-methoxybenzyl)propyl-sulfonamido)phenyl)-3-fluoroisonicotinamide was prepared according to the general procedure as described for Example 63, step A, substituting N-(3-amino-4-chloro-2-fluorophenyl)-3-fluoro-N-(4-methoxybenzyl)propane-1-sulfonamide for N-(3-amino-2,4-difluorophenyl)-N-(4-methoxybenzyl)propane-1-sulfonamide. ¹H NMR (400 MHz, CDCl₃) δ 8.42 (d, J=4.9 Hz, 1H), 8.03 (d, J=11.3 Hz, 1H), 7.94 (t, J=4.5 Hz, 1H), 7.23-7.13 (m, 3H), 7.07-7.00 (m, 1H), 6.81 (t, J=5.7 Hz, 2H), 4.77 (s, 2H), 4.63 (t, J=5.7 Hz, 1H), 4.51 (t, J=5.7 Hz, 1H), 3.76 (s, 3H), 3.29-3.21 (m, 2H), 2.38-2.20 (m, 2H). LC/MS: m/z 562.0 [M+1].

Step B:

Using a similar procedure as described for Example 63, step B, substituting 2-chloro-N-(6-chloro-2-fluoro-3-(3-fluoro-N-(4-methoxybenzyl)propyl-sulfonamido)phenyl)-3-fluoroisonicotinamide for 2-chloro-N-(2-chloro-6-fluoro-3-(N-(4-methoxybenzyl)propyl-sulfonamido)phenyl)-3-fluoroisonicotinamide, afforded the following two compounds: N-(6-chloro-2-fluoro-3-(3-fluoro-N-(4-methoxybenzyl)propyl-sulfonamido)phenyl)-2-cyano-3-fluoroisonicotinamide; ¹H NMR (400 MHz, CDCl₃) δ 8.78 (d, J=4.7 Hz, 1H), 8.29 (t, J=5.1 Hz, 1H), 8.00 (d, J=11.0 Hz, 1H), 7.21-7.14 (m, 3H), 7.05 (t, J=8.2 Hz, 1H), 6.80 (d, J=8.6 Hz, 2H), 4.77 (s, 2H), 4.63 (t, J=5.6 Hz, 1H), 4.52 (t, J=5.6 Hz, 1H), 3.77 (s, 3H), 3.31-3.22 (m, 2H), 2.37-2.20 (m, 2H); LC/MS: m/z 553.0 [M+1], RT=2.89 min; 2-cyano-N-(6-cyano-2-fluoro-3-(3-fluoro-N-(4-methoxy-benzyl)propyl-sulfonamido)phenyl)-3-fluoroisonicotinamide; ¹H NMR (400 MHz, CDCl₃) δ 8.78 (d, J=4.8 Hz, 1H), 8.32 (d, J=10.2 Hz, 1H), 8.27 (t, J=5.2 Hz, 1H), 7.41 (d, J=8.5 Hz, 1H), 7.22 (t, J=7.2 Hz, 1H), 7.16 (d, J=8.6 Hz, 2H), 6.80 (d, J=8.6 Hz, 2H), 4.79 (s, 2H), 4.62 (t, J=5.6 Hz, 1H), 4.51 (t, J=5.6 Hz, 1H), 3.77 (s, 3H), 3.30-3.22 (m, 2H), 2.36-2.19 (m, 2H).

Step C:

4-Amino-N-(6-cyano-2-fluoro-3-(3-fluoro-N-(4-methoxybenzyl)propyl-sulfonamido)phenyl)-pyrido[3,2-d]pyrimidine-8-carboxamide was prepared following the general procedure as described for Example 63, step C, substituting 2-cyano-N-(6-cyano-2-fluoro-3-(3-fluoro-N-(4-methoxybenzyl)propyl-sulfonamido)phenyl)-3-fluoroisonicotinamide for 4-amino-N-(2-chloro-6-fluoro-3-(N-(4-methoxybenzyl)propyl-sulfonamido)phenyl)pyrido[3,2-d]pyrimidine-8-carboxamide. LC/MS: m/z 568.2 [M+1].

Step D:

To a stirred mixture of 4-amino-N-(6-cyano-2-fluoro-3-(3-fluoro-N-(4-methoxybenzyl)propylsulfonamido)phenyl)pyrido[3,2-d]pyrimidine-8-carboxamide (83 mg, 0.146 mmol) in DCM (5 mL) was added trifluoroacetic acid (3 mL). The reaction mixture was stirred at room temperature overnight and then concentrated under reduced pressure. The crude product was partitioned between saturated aqueous NaHCO₃ solution and EtOAc, and the organic layer was washed with brine, dried over Na₂SO₄, filtered, and concentrated under reduced pressure. Crystallization from methanol afforded the title compound (14.2 mg, 20.9%) as a solid. ¹H NMR (400 MHz, DMSO-d₆) δ 13.06 (s, 1H), 10.09 (s, 1H), 8.98 (d, J=4.5 Hz, 1H), 8.54 (s, 1H), 8.48-8.41 (m, 2H), 8.38 (s, 1H), 7.97 (s, 1H), 7.46 (s, 1H), 7.41 (s, 2H), 4.61 (t, J=6.0 Hz, 1H), 4.49 (t, J=6.0 Hz, 1H), 3.29-3.22 (m, 2H), 2.21-2.03 (m, 2H); LC/MS: m/z 466.1 [M+1].

### Example 69

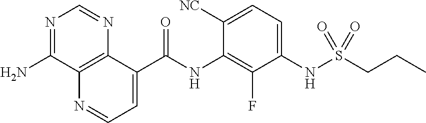 [SMILES: CCCS(=O)(=O)Nc1ccc(C#N)c(NC(=O)c2ccnc3c(N)ncnc23)c1F]

### 4-Amino-pyrido[3,2-d]pyrimidine-8-carboxylic acid [6-cyano-2-fluoro-3-(propane-1-sulfonylamino)-phenyl]-amide

Step A:

Using similar procedures as described for Example 68, steps A to C, substituting N-(3-amino-4-chloro-2-fluorophenyl)-N-(4-methoxybenzyl)propane-1-sulfon-amide for N-(3-amino-2,4-difluorophenyl)-N-(4-methoxybenzyl)propane-1-sulfonamide, gave 4-amino-N-(6-cyano-2-fluoro-3-(N-(4-methoxybenzyl)propylsulfonamido)phenyl)-pyrido[3,2-d]pyrimidine-8-carboxamide as a solid. ¹H NMR (400 MHz, CDCl₃) δ 13.61 (s, 1H), 8.98 (d, J=4.5 Hz, 1H), 8.76-8.60 (m, 2H), 7.40-7.35 (m, 1H), 7.31 (broad s, 1H), 7.21 (d, J=8.6 Hz, 2H), 7.16-7.10 (m, 1H), 6.80 (t, J=8.6 Hz, 2H), 6.33 (broad s, 1H), 4.81 (s, 2H), 3.77 (s, 3H), 3.15-3.06 (m, 2H), 2.00-1.88 (m, 2H), 1.07 (t, J=7.4 Hz, 3H). LC/MS: m/z 550.1 [M+1].

Step B:

Using a similar procedure as described for Example 68, step D, substituting 4-amino-N-(6-cyano-2-fluoro-3-(N-(4-methoxybenzyl)propyl sulfonamido)-phenyl)pyrido[3,2-d]pyrimidine-8-carboxamide for N-(6-cyano-2-fluoro-3-(3-fluoro-N-(4-methoxybenzyl)propylsulfonamido)phenyl)pyrido[3,2-d]pyrimidine-8-carboxamide, afforded the title compound as a solid. ¹H NMR (400 MHz, DMSO-d₆) δ 13.04 (s, 1H), 9.00 (d, J=4.5 Hz, 1H), 8.58 (s, 1H), 8.51 (broad s, 1H), 8.49-8.40 (m, 2H), 8.13 (s, 1H), 7.38 (s, 2H), 2.92 (s, 2H), 1.74-1.63 (m, 2H), 0.94 (t, J=7.4 Hz, 3H).; LC/MS: m/z 430.1 [M+1].

### Example 70

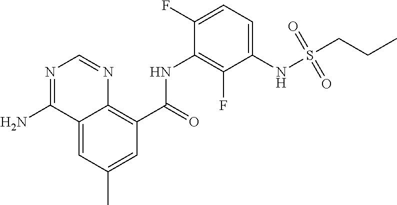 [SMILES: CCCS(=O)(=O)Nc1ccc(F)c(NC(=O)c2cc(C)cc3c(N)ncnc23)c1F]

### 4-Amino-6-methyl-quinazoline-8-carboxylic acid [2,6-difluoro-3-(propane-1-sulfonylamino)-phenyl]-amide

Step A:

To a suspension of 4-hydroxy-6-methylquinazoline-8-carboxylic acid (2.50 g, 12.0 mmol) in thionyl chloride (50 mL) was added DMF (0.19 mL, 2.45 mmol). The reaction mixture was heated at reflux for 2 hours and then concentrated in vacuo. The residue was redissolved in chloroform and concentrated in vacuo. The resultant solid was dried under high vacuum to afford 4-chloro-6-methylquinazoline-8-carbonyl chloride.

Step B:

To a solution of N-(3-amino-2,4-difluorophenyl)propane-1-sulfonamide (500.0 mg, 2.00 mmol) in chloroform (10.0 mL) was added 4 Å molecular sieves, pyridine (0.162 mL, 2.00 mmol), and 4-chloro-6-methylquinazoline-8-carbonyl chloride (578 mg, 2.40 mmol). The reaction mixture was stirred at room temperature for 2 hours and filtered. The filtrate was diluted with dichloromethane and washed with a saturated aqueous solution of NaHCO₃. The aqueous layer was extracted twice with dichloromethane, and the combined organic layers were dried with MgSO₄, filtered, and concentrated in vacuo. The crude product was passed through a short column and was used in the next step without further purification. ¹H NMR (500 MHz, DMSO-d₆) δ 11.75 (s, 1H), 9.70 (s, 1H), 9.22 (s, 1H), 8.65 (s, 1H), 8.43-8.40 (s, 1H), 7.40 (tt, 1H), 7.23 (m, 1H), 3.17-3.03 (m, 2H), 2.68 (s, 3H), 1.85-1.72 (m, 2H), 1.05-0.93 (m, 3H). LC/MS: m/z 455.1 [M+1].

Step C:

A 20-mL microwave vessel was charged with 4-chloro-N-(2,6-difluoro-3-(propylsulfonamido)phenyl)-6-methylquinazoline-8-carboxamide (414 mg, 0.91 mmol) and isopropanol (10 mL). Ammonia gas was passed through the reaction mixture for 10 minutes. The vessel was capped and the reaction mixture subjected to microwave irradiation at 105° C. for 15 minutes. The reaction mixture was concentrated in vacuo and the crude product was (triturated twice with 10% isopropanol in water. The solid was filtered and dried under high vacuum to afford 4-amino-6-methyl-quinazoline-8-carboxylic acid [2,6-difluoro-3-(propane-1-sulfonylamino)-phenyl]-amide (357 mg, 90%) as an off-white solid. ¹H NMR (400 MHz, DMSO-d₆) δ 13.30 (s, 1H), 9.73 (s, 1H), 8.60-8.45 (m, 2H), 8.35 (s, 1H), 8.21 (d, 2H), 7.36 (td, 1H), 7.23 (t, 1H), 3.15-2.98 (m, 2H), 2.53 (s, 3H), 1.86-1.66 (m, 2H), 1.03-0.88 (m, 3H). LC/MS: m/z 436.2 (100%) [M+1].

### Example 71

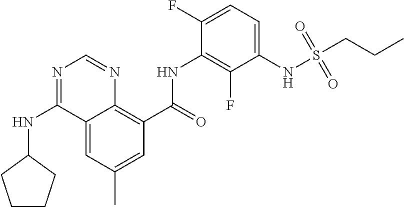 [SMILES: CCCS(=O)(=O)Nc1ccc(F)c(NC(=O)c2cc(C)cc3c(NC4CCCC4)ncnc23)c1F]

### 4-Cyclopentylamino-6-methyl-quinazoline-8-carboxylic acid [2,6-difluoro-3-(propane-1-sulfonylamino)-phenyl]-amide

To a suspension of 4-chloro-N-(2,6-difluoro-3-(propylsulfonamido)phenyl)-6-methylquinazoline-8-carboxamide (35 mg, 0.077 mmol) in isopropanol (2 mL) was added N,N-diisopropylethylamine (49.7 mg, 0.385 mmol) and cyclopentanamine (32.8 mg, 0.385 mmol). The reaction mixture was heated at 85° C. for 4 hours and then concentrated in vacuo. The crude product was purified by reverse phase HPLC to give 4-(cyclopentylamino)-N-(2,6-difluoro-3-(propylsulfonamido)phenyl)-6-methylquinazoline-8-carboxamide (20 mg, 40%) as a white solid. ¹H NMR (400 MHz, DMSO-d₆) δ 13.22 (s, 1H), 8.60 (s, 1H), 8.48 (s, 2H), 8.35 (d, 1H), 7.32 (m, 1H), 7.12 (m, 1H), 4.63 (m, 1H), 3.05-2.90 (m, 2H), 2.15-1.93 (m, 2H), 1.86-1.49 (m, 8H), 0.96 (t, 3H). LC/MS: m/z 504.2 (100%) [M+1].

Examples 72-76 listed in Table 6 were prepared applying the procedure described in Example 71 and using appropriate amino building blocks.

TABLE 6

| | | | | ¹H NMR δ |
|---|---|---|---|---|
| | Structure | Name | MS m/z | (400 MHz, DMSO-d₆) |
| 72 | 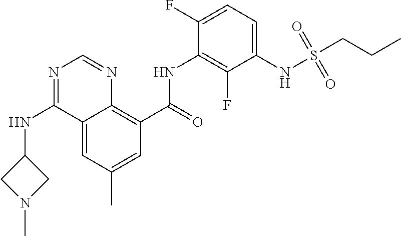 [SMILES: CCCS(=O)(=O)Nc1ccc(F)c(NC(=O)c2cc(C)cc3c(NC4CN(C)C4)ncnc23)c1F] | 6-Methyl-4-(1-methyl- azetidin-3-ylamino)- quinazoline-8-carboxylic acid [2,6-difluoro-3- (propane-1- sulfonylamino)-phenyl]- amide | 505.1 | 12.00 (s, 1H), 9.64 (s, 1H), 9.31 (s, 1H), 8.85 (dd, 1H), 8.60 (dd, 1H), 8.07 (m, 1H), 7.56-7.31 (m, 2H), 3.22- 3.01 (m, 2H), 1.89-1.69 (m, 2H), 1.07-0.89 (m, 3H). |
| 73 | 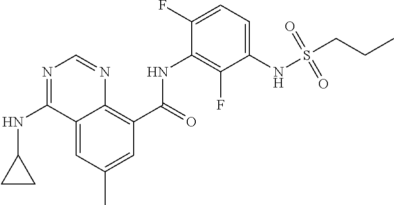 [SMILES: CCCS(=O)(=O)Nc1ccc(F)c(NC(=O)c2cc(C)cc3c(NC4CC4)ncnc23)c1F] | 4-Cyclopropylamino-6- methyl-quinazoline-8- carboxylic acid [2,6- difluoro-3-(propane-1- sulfonylamino)-phenyl]- amide | 476.1 | 13.23 (s, 1H), 9.88 (s, 1H), 8.66 (s, 1H), 8.60 (d, 1H), 8.48 (s, 1H), 8.36 (s, 1H), 7.35 (m, 1H), 7.18 (m, 1H), 3.12 (m, 1H), 3.08-2.97 (m, 2H), 1.84-1.64 (m, 2H), 0.98 (t, 3H), 0.90-0.79 (m, 2H), 0.77-0.62 (m, 2H) |
| 74 | 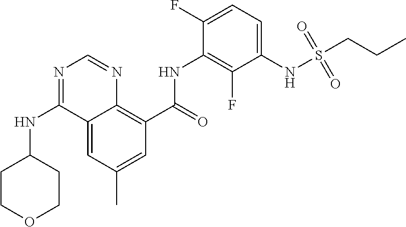 [SMILES: CCCS(=O)(=O)Nc1ccc(F)c(NC(=O)c2cc(C)cc3c(NC4CCOCC4)ncnc23)c1F] | 6-Methyl-4-(tetrahydro- pyran-4-ylamino)- quinazoline-8-carboxylic acid [2,6-difluoro-3- (propane-1- sulfonylamino)-phenyl]- amide | 520.2 | 13.24 (s, 1H), 9.75 (s, 1H), 8.61 (s, 1H), 8.42 (m, 3H), 7.48-7.23 (m, 1H), 7.20 (m, 1H), 4.48 (m, 1H), 4.12- 3.77 (m, 2H), 3.46 (t, 2H), 3.19-2.91 (m, 2H), 2.56 (s, 3H), 2.10-1.81 (m, 2H), 1.79-1.55 (m, 4H), 0.98 (t, 3H) |
| 75 | 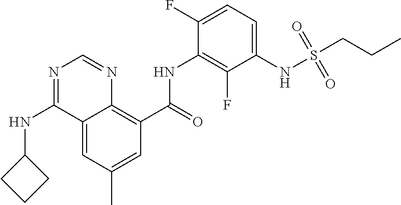 [SMILES: CCCS(=O)(=O)Nc1ccc(F)c(NC(=O)c2cc(C)cc3c(NC4CCC4)ncnc23)c1F] | 4-Cyclobutylamino-6- methyl-quinazoline-8- carboxylic acid [2,6- difluoro-3-(propane-1- sulfonylamino)-phenyl]- amide | 490.1 | 13.22 (s, 1H), 9.78 (s, 1H), 8.70 (m, 1H), 8.58 (s, 1H), 8.47 (m, 2H), 7.34 (m, 1H), 7.18 (m, 1H), 4.77 (dd, J = 15.7, 8.1 Hz, 1H), 3.13-2.95 (m, 2H), 2.56 (s, 3H), 2.36 (dd, J = 17.7, 9.1 Hz, 2H), 2.27-2.10 (m, 2H), 1.85- 1.68 (m, 4H), 0.97 (t, 3H) |
| 76 | 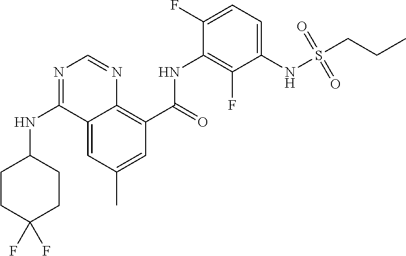 [SMILES: CCCS(=O)(=O)Nc1ccc(F)c(NC(=O)c2cc(C)cc3c(NC4CCC(F)(F)CC4)ncnc23)c1F] | 4-(4,4-Difluoro- cyclohexylamino)-6- methyl-quinazoline-8- carboxylic acid [2,6- difluoro-3-(propane-1- sulfonylamino)-phenyl]- amide | 554.3 | 13.20 (s, 1H), 9.89 (s, 1H), 8.62 (s, 1H), 8.47 (m, 2H), 8.40-8.19 (m, 1H), 7.34 (m, 1H), 7.17 (m, 1H), 4.46 (bs, 1H), 3.01 (m, 2H), 2.55 (s, 3H), 2.12 (m, 2H), 2.03 (m, 4H), 1.73 (m, 4H), 0.97 (t, 3H) |

### Example 77

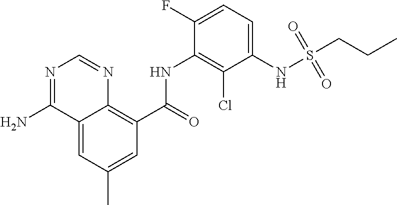 [SMILES: CCCS(=O)(=O)Nc1ccc(F)c(NC(=O)c2cc(C)cc3c(N)ncnc23)c1Cl]

### 4-Amino-6-methyl-quinazoline-8-carboxylic acid [2-chloro-6-fluoro-3-(propane-1-sulfonylamino)-phenyl]-amide

Step A:

To a stirred solution of N-(3-Amino-2-chloro-4-fluorophenyl)-N-(4-methoxybenzyl)propane-1-sulfonamide (500 mg, 1.29 mmol) in toluene (5 mL, 50 mmol) was slowly added 2M trimethylaluminum in hexane (678 uL, 1 mmol). The reaction mixture was stirred at room temperature for 1 hour and methyl 4-chloro-6-methylquinazoline-8-carboxylate (306 mg, 1.29 mmol) was added. The resulting mixture was stirred at 80° C. for 2 days, cooled to room temperature and quenched with an aqueous solution of potassium sodium tartrate (1N, 50 mL). This mixture was stirred at room temperature for 1 hour and poured into EtOAc. The organic layer was washed with brine, dried over MgSO₄, filtered and evaporated under reduced pressure. The crude product was purified using flash column chromatography (0-50% ethyl acetate: heptane) to give 4-chloro-N-(2-chloro-6-fluoro-3-(N-(4-methoxybenzyl)propylsulfonamido)phenyl)-6-methylquinazoline-8-carboxamide (236 mg, 31%). ¹H NMR (500 MHz, DMSO-d6) δ 12.00 (s, 1H), 9.24 (s, 1H), 8.69 (s, 1H), 8.38 (s, 1H), 7.31 (p, 2H), 7.17 (d, J=8.6 Hz, 2H), 6.84 (d, J=8.6 Hz, 2H), 4.86 (d, J=13.7 Hz, 1H), 4.61 (d, J=14.7 Hz, 1H), 3.71 (s, 3H), 3.39-3.15 (m, 2H), 2.66 (s, 3H), 1.81 (m, 2H), 1.02 (t, 3H). LC/MS: m/z 591.1 (100%) [M+1].

Step B:

Using a similar procedure as described for Example 70, step C, substituting 4-chloro-N-(2-chloro-6-fluoro-3-(N-(4-methoxybenzyl)propylsulfonamido)phenyl)-6-methyl-quinazoline-8-carboxamide for 4-chloro-N-(2,6-difluoro-3-(propylsulfonamido)phenyl)-6-methylquinazoline-8-carboxamide, gave 4-amino-N-(2-chloro-6-fluoro-3-(N-(4-methoxy-benzyl)propylsulfonamido)phenyl)-6-methylquinazoline-8-carboxamide as an off-white solid. LC/MS: m/z 572.2 [M+1].

Step C:

To 4-amino-N-(2-chloro-6-fluoro-3-(N-(4-methoxybenzyl)propylsulfonamido)-phenyl)-6-methylquinazoline-8-carboxamide (75 mg, 0.13 mmol) dissolved in methylene chloride (4 mL, 60 mmol) was added trifluoroacetic acid (2 mL, 25 mmol). The reaction mixture was stirred at room temperature for 18 hours and the volatile solvent was concentrated. The resulting oil was redissolved in ethyl acetate and washed with water. The organic layer was dried over magnesium sulfate, filtered, and evaporated in vacuo. Trituration with ether afforded 4-amino-6-methyl-quinazoline-8-carboxylic acid [2-chloro-6-fluoro-3-(propane-1-sulfonylamino)-phenyl]-amide (50 mg, 80%). ¹H NMR (500 MHz, DMSO-d₆) δ 9.55 (s, 1H), 8.51-8.35 (m, 2H), 8.16 b(s, 2H), 7.54-7.27 (m, 2H), 3.18-3.07 (m, 2H), 2.53 (s, 3H), 1.85-1.72 (m, 2H), 0.99 (t, 3H). LC/MS: m/z 452.1 [M+1].

### Example 78

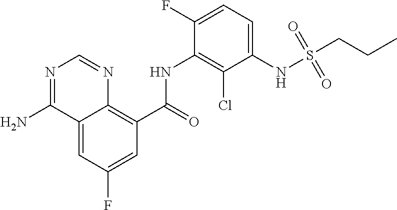 [SMILES: CCCS(=O)(=O)Nc1ccc(F)c(NC(=O)c2cc(F)cc3c(N)ncnc23)c1Cl]

### 4-Amino-6-fluoro-quinazoline-8-carboxylic acid [2-chloro-6-fluoro-3-(propane-1-sulfonylamino)-phenyl]-amide

Step A:

Using a similar procedure as described for Example 70, step A, substituting 6-fluoro-4-hydroxyquinazoline-8-carboxylic acid for 4-hydroxy-6-methylquinazoline-8-carboxylic acid, provided 4-chloro-6-fluoroquinazoline-8-carbonyl chloride which was used in subsequent reaction without further purification.

Step B:

Using a similar procedure as described in Example 70, step B, substituting 4-chloro-6-fluoroquinazoline-8-carbonyl chloride for 4-chloro-6-methylquinazoline-8-carbonyl chloride, afforded 4-chloro-N-(2-chloro-6-fluoro-3-(propylsulfonamido)phenyl)-6-fluoroquinazoline-8-carboxamide as an off-white solid which was used in the next step without further purification. LC/MS: m/z 475.1 [M+1].

Step C:

Using a similar procedure as described in Example 70, step C, substituting 4-chloro-N-(2-chloro-6-fluoro-3-(propylsulfonamido)phenyl)-6-fluoroquinazoline-8-carboxamide for 4-chloro-N-(2,6-difluoro-3-(propylsulfonamido)phenyl)-6-methyl-quinazoline-8-carboxamide afforded 4-amino-6-fluoro-quinazoline-8-carboxylic acid [2-chloro-6-fluoro-3-(propane-1-sulfonylamino)-phenyl]amide (80 mg, 60%) as a white solid. ¹H NMR (500 MHz, DMSO-d₆) δ 13.42 (s, 1H), 9.57 (s, 1H), 8.58 (s, 1H), 8.40 (s, 2H), 8.31 (s, 2H), 7.46 (m, 1H), 7.38 (m, 1H), 3.19-3.06 (m, 2H), 1.78 (m, 2H), 0.99 (t, 3H). LC/MS: m/z 456.0 [M+1].

### Example 79

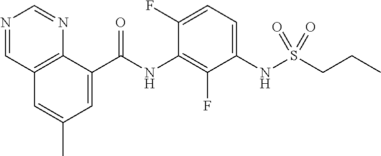 [SMILES: CCCS(=O)(=O)Nc1ccc(F)c(NC(=O)c2cc(C)cc3cncnc23)c1F]

### 6-Methyl-quinazoline-8-carboxylic acid [2,6-difluoro-3-(propane-1-sulfonylamino)-phenyl]-amide

Using a similar procedure as described in Example 54, substituting 4-amino-6-methyl-quinazoline-8-carboxylic acid [2,6-difluoro-3-(propane-1-sulfonylamino)-phenyl]-amide for quinazoline-8-carboxylic acid [2,6-difluoro-3-(propane-1-sulfonylamino)-phenyl]amide gave the title compound as a white solid. ¹H NMR (500 MHz, DMSO-d₆) δ 12.15 (s, 1H), 9.75 (s, 2H), 9.45 (s, 1H), 8.67 (d, J=1.8 Hz, 1H), 8.26 (s, 1H), 7.50-7.32 (m, 1H), 7.26 (t, J=9.0 Hz, 1H), 3.16-3.01 (m, 2H), 2.64 (s, 3H), 1.86-1.69 (m, 2H), 0.99 (t, J=7.4 Hz, 3H). LC/MS: m/z 421.0 [M+1].

Table 7 shows the activity of certain compounds of the invention tested in the above B-RAF V600E inhibition assay (Example A).

TABLE 7

| Example | BRAF V600E IC₅₀ (μM) |
|---|---|
| 1 | 0.00177 |
| 2 | 0.00125 |
| 3 | 0.00053 |
| 4 | 0.19100 |
| 5 | 0.00477 |
| 6 | 0.05040 |
| 7 | 0.00007 |
| 8 | 0.01083 |
| 9 | 0.00039 |
| 10 | 0.00055 |
| 11 | 0.00413 |
| 12 | 0.00155 |
| 13 | 0.00380 |
| 14 | 0.02790 |
| 15 | 0.00050 |
| 16 | 0.00260 |
| 17 | 0.00058 |
| 18 | 0.00153 |
| 19 | 0.00497 |
| 20 | 0.00224 |
| 21 | 0.01851 |
| 22 | 0.06489 |
| 23 | 0.00028 |
| 24 | 0.00014 |
| 25 | 0.00009 |
| 26 | 0.00031 |
| 27 | 0.00008 |
| 28 | 0.00011 |
| 29 | 0.00008 |
| 30 | 0.00008 |
| 31 | 0.00040 |
| 32 | 0.00038 |
| 33 | 0.00017 |
| 34 | 0.01270 |
| 35 | 0.00016 |
| 36 | 0.01477 |
| 37 | 0.02148 |
| 38 | 0.00701 |
| 39 | 0.00750 |
| 40 | 0.00220 |
| 41 | >1.0 |
| 42 | >1.0 |
| 43 | 0.29273 |
| 44 | >1.0 |
| 45 | 0.54536 |
| 46 | >1.0 |
| 47 | 0.10471 |
| 48 | >1.0 |
| 49 | 0.73700 |
| 50 | >1.0 |
| 51 | 0.14288 |
| 52 | 0.28975 |
| 53 | 0.97628 |
| 54 | 0.11880 |
| 55 | 0.03221 |
| 56 | 0.20377 |
| 57 | 0.00096 |
| 58 | 0.01711 |
| 59 | 0.00149 |
| 60 | 0.00082 |
| 61 | 0.01926 |
| 62 | 0.00307 |
| 63 | 0.00101 |
| 64 | 0.00311 |
| 65 | 0.00114 |
| 66 | 0.00075 |
| 67 | 0.00042 |
| 68 | 0.01454 |
| 69 | 0.01948 |
| 70 | 0.00140 |
| 71 | >1.0 |
| 72 | — |
| 73 | 0.87940 |
| 74 | >1.0 |
| 75 | >1.0 |
| 76 | >1.0 |
| 77 | 0.00045 |
| 78 | 0.00049 |
| 79 | 0.05884 |

Table 8 shows the activity of certain compounds of the invention tested in the above cellular ERK 1/2 phosphorylation assay (Example A1).

TABLE 8

| | P-ERK Malme3-M |
|---|---|
| Example | IC₅₀ (μM) |
| 1 | 0.00867 |
| 2 | 0.00960 |
| 3 | 0.00723 |
| 4 | 0.43270 |
| 5 | 0.04074 |
| 6 | 1.72420 |
| 7 | 0.00720 |
| 8 | 0.03264 |
| 9 | 0.01204 |
| 10 | 0.00673 |
| 11 | 0.04502 |
| 12 | 0.00854 |
| 13 | 0.01487 |
| 14 | 0.38130 |
| 15 | 0.00608 |
| 16 | 0.01517 |
| 17 | 0.00870 |
| 18 | 0.01818 |
| 19 | 0.02590 |
| 20 | 0.03237 |
| 21 | 1.31620 |
| 22 | 1.10600 |
| 23 | 0.00389 |
| 24 | 0.00405 |
| 25 | 0.00221 |
| 26 | 0.01079 |
| 27 | 0.00407 |
| 28 | 0.01444 |
| 29 | 0.02531 |
| 30 | 0.00710 |
| 31 | 0.04199 |
| 32 | 0.01770 |
| 33 | 0.01252 |
| 34 | 0.01564 |
| 35 | 0.00748 |
| 36 | 0.02522 |
| 37 | 0.03973 |
| 38 | 0.02804 |
| 39 | 0.03269 |
| 40 | 0.15809 |
| 41 | — |
| 42 | — |
| 43 | 2.90980 |
| 44 | — |
| 45 | — |
| 46 | — |
| 47 | 1.16770 |
| 48 | — |
| 49 | — |
| 50 | — |
| 51 | 2.30810 |
| 52 | 5.29450 |
| 53 | — |
| 54 | 0.52650 |
| 55 | 0.09282 |
| 56 | 0.63790 |
| 57 | 0.08758 |
| 58 | 0.30095 |
| 59 | 0.08370 |
| 60 | 0.05340 |
| 61 | 0.11629 |
| 62 | 0.04400 |
| 63 | 0.00760 |
| 64 | 0.00970 |
| 65 | 0.00940 |
| 66 | 0.00304 |
| 67 | 0.00280 |
| 68 | 0.12380 |
| 69 | 0.30650 |
| 70 | 0.00859 |
| 71 | — |
| 72 | — |
| 73 | — |
| 74 | — |
| 75 | — |
| 76 | — |
| 77 | 0.00783 |
| 78 | 0.01567 |
| 79 | 0.25360 |

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

Specific reference is made to U.S. Provisional Patent Appl. Nos. 61/312,448 filed Mar. 10, 2010 and 61/238,105 filed Aug. 28, 2009, which are both incorporated herein by reference in their entirety for all purposes.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound selected from Formula I: 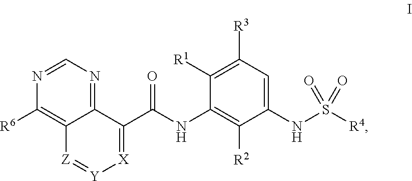
stereoisomers, tautomers, prodrugs and pharmaceutically acceptable salts thereof, wherein:
X is N or CR¹²;
Y is N or CR¹³;
Z is N or CR¹⁴, wherein no more than two of X, Y and Z can be N at the same time;
R¹ and R² are independently selected from hydrogen, halogen, CN, —C(O)NR⁶R⁷, C₁-C₃ alkyl, C₁-C₃ alkenyl, C₂-C₃ alkynyl and C₁-C₃ alkoxy;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, 3-6 membered heterocyclyl, a 5-6 membered heteroaryl or NR⁶R⁷, wherein the cycloalkyl, alkyl, alkenyl, alkynyl, phenyl, heterocyclyl and heteroaryl are optionally substituted with OR¹⁵, halogen, phenyl, C₃-C₄ cycloalkyl or C₁-C₄ alkyl optionally substituted with halogen;
R⁵ is hydrogen, C₁-C₆ alkyl or NR⁸R⁹;
R⁶ and R⁷ are each independently hydrogen or C₁-C₆ alkyl optionally substituted by halogen; or
R⁶ and R⁷ are independently taken together with the atom to which they are attached to form a 3-6 membered heterocyclyl optionally substituted by halogen, oxo or C₁-C₃ alkyl;
R⁸ is hydrogen;
R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)3-6-membered heterocyclyl or (C₀-C₃ alkyl)5-6-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;
R¹⁰ and R¹¹ are independently hydrogen or C₁-C₆ alkyl optionally substituted by halogen; or
R¹⁰ and R¹¹ are taken together with the atom to which they are attached to form a 3-6 membered heterocyclyl optionally substituted by halogen, oxo or C₁-C₃ alkyl;
R¹² is hydrogen, C₁-C₃ alkyl or halogen;
R¹³ is hydrogen, C₁-C₆ alkyl, C₂-C₆ alkenyl, C₂-C₆ alkynyl or halogen, wherein said alkyl, alkenyl and alkynyl are optionally substituted by OR¹⁸;
R¹⁴ is hydrogen, C₁-C₃ alkyl or halogen;
R¹⁵ is hydrogen or C₁-C₃ alkyl optionally substituted by halogen;
R¹⁶ and R¹⁷ are independently hydrogen or C₁-C₆ alkyl optionally substituted by halogen; or
R¹⁶ and R¹⁷ are taken together with the atom to which they attached to form a 3-6 membered heterocyclyl optionally substituted by halogen, oxo or C₁-C₃ alkyl; and
R¹⁸ is hydrogen or C₁-C₃ alkyl.

2. The compound of claim 1, wherein:
X is N or CR¹²;
Y is N or CR¹³;
Z is N or CR¹⁴, wherein no more than two of X, Y and Z can be N at the same time;
R¹ and R² are independently selected from hydrogen, halogen, CN, C₁-C₃ alkyl and C₁-C₃ alkoxy;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, a 5-6 membered heteroaryl or NR⁶R⁷, wherein the cycloalkyl, alkyl, alkenyl, alkynyl, phenyl and heteroaryl are optionally substituted with OR¹⁵, halogen, phenyl, C₃-C₄ cycloalkyl, or C₁-C₄ alkyl optionally substituted with halogen;
R⁵ is hydrogen or NR⁸R⁹;
R⁶ and R⁷ are each independently hydrogen or C₁-C₆ alkyl optionally substituted by halogen; or
R⁶ and R⁷ are independently taken together with the atom to which they are attached to form a 3-6 membered heterocyclyl optionally substituted by halogen, oxo or C₁-C₃ alkyl;
R⁸ is hydrogen;
R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)3-6-membered heterocyclyl or (C₀-C₃ alkyl)5-6-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;
R¹⁰ and R¹¹ are independently hydrogen or C₁-C₆ alkyl optionally substituted by halogen; or
R¹⁰ and R¹¹ are taken together with the atom to which they attached to form a 3-6 membered heterocyclyl optionally substituted by halogen, oxo or C₁-C₃ alkyl;
R¹² is hydrogen, C₁-C₃ alkyl or halogen;
R¹³ is hydrogen, C₁-C₃ alkyl or halogen;
R¹⁴ is hydrogen, C₁-C₃ alkyl or halogen;
R¹⁵ is hydrogen or C₁-C₃ alkyl optionally substituted by halogen; and
R¹⁶ and R¹⁷ are independently hydrogen or C₁-C₆ alkyl optionally substituted by halogen; or
R¹⁶ and R¹⁷ are taken together with the atom to which they attached to form a 3-6 membered heterocyclyl optionally substituted by halogen, oxo or C₁-C₃ alkyl.

3. A compound of claim 1, wherein X is CR¹² and Z is CR¹⁴.

4. A compound of claim 1, wherein X is CH and Z is CH.

5. A compound of claim 1, wherein X, Y and Z are CH.

6. A compound of claim 1, wherein X is CR¹²; Z is CR¹⁴ and Y is N.

7. A compound of claim 1, wherein X and Z are CH and Y is N.

8. A compound of claim 1, wherein R¹, R² and R³ are independently selected from hydrogen, halogen or C₁-C₃ alkyl; R⁴ is C₃-C₄ cycloalkyl or C₁-C₆ alkyl optionally substituted with OH, halogen or C₃-C₄ cycloalkyl; and R⁵ is hydrogen or NHR⁹.

9. (canceled)

10. A compound of claim 1, wherein the residue: 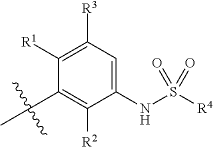
of Formula I is selected from: 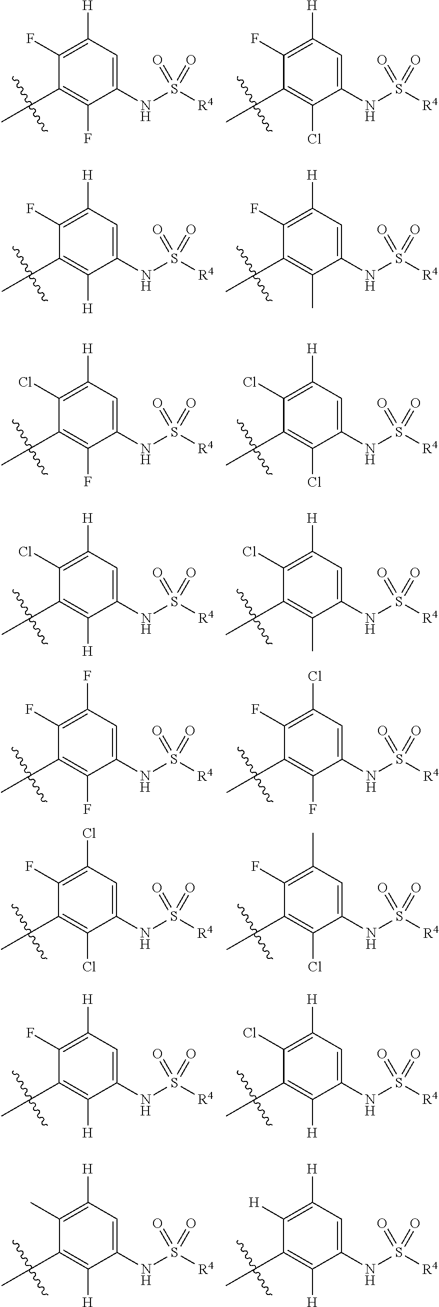 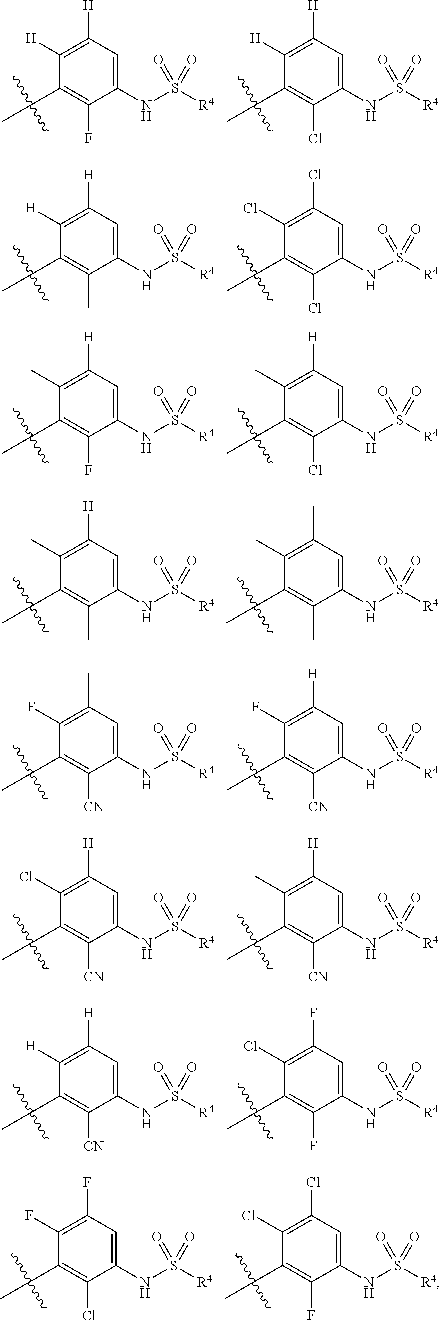
wherein the wavy lines represent the point of attachment of the residue in Formula I.

11-21. (canceled)

22. A compound of claim 1, wherein R⁴ is cyclopropyl, ethyl, propyl, butyl, isobutyl, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃—N(CH₃)CH₂CH₃, —N(CH₃)₂, or pyrrolidinyl.

23-26. (canceled)

27. A compound of claim 1, wherein R⁵ is NHR⁹; and R⁹ is hydrogen, C₁-C₃ alkyl optionally substituted by halogen, C₃-C₆ cycloalkyl optionally substituted by halogen or 3-6 membered heterocyclyl optionally substituted by C₁-C₃ alkyl.

28. A compound of claim 1, wherein R⁵ is NHR⁹; and R⁹ is hydrogen, 2-fluoroethyl, cyclopropyl, cyclobutyl, cyclopentyl, cyclohexyl, 4,4-difluorocyclohexyl, N-methylazetidinyl, morpholinyl, tetrahydropyranyl or piperidinyl.

29. (canceled)

30. A pharmaceutical composition, comprising a compound of claim 1, and a pharmaceutically acceptable carrier or excipient.

31. A method of preventing or treating a disease or disorder modulated by b-Raf, comprising administering to a mammal in need of such treatment an effective amount of a compound of claim 1.

32. A method of preventing or treating cancer, comprising administering to a mammal in need of such treatment an effective amount of a compound of claim 1, alone or in combination with one or more additional compounds having anti-cancer properties.

33. The method of claim 32, wherein the cancer is a sarcoma.

34. The method of claim 32, wherein the cancer is a carcinoma.

35. The method of claim 32, wherein the carcinoma is squamous cell carcinoma.

36. The method of claim 32, wherein the carcinoma is an adenoma or adenocarcinoma.

37. The method of claim 32, wherein the cancer is breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's or leukemia.

38-43. (canceled)

44. A compound of claim 1, wherein:
X is N or CR¹²;
Y is N or CR¹³;
Z is N or CR¹⁴, wherein no more than two of X, Y and Z can be N at the same time;
R¹ and R² are independently selected from hydrogen, halogen, CN, C₁-C₃ alkyl, C₁-C₃ alkenyl, C₁-C₃ alkynyl and C₁-C₃ alkoxy;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, a 5-6 membered heteroaryl or NR⁶R⁷, wherein the cycloalkyl, alkyl, alkenyl, alkynyl, phenyl and heteroaryl are optionally substituted with OR¹⁵, halogen, phenyl, C₃-C₄ cycloalkyl or C₁-C₄ alkyl optionally substituted with halogen;
R⁵ is hydrogen or NR⁸R⁹;
R⁶ and R⁷ are each independently hydrogen or C₁-C₆ alkyl optionally substituted by halogen; or
R⁶ and R⁷ are independently taken together with the atom to which they are attached to form a 3-6 membered heterocyclyl optionally substituted by halogen, oxo or C₁-C₃ alkyl;
R⁸ is hydrogen;
R⁹ is hydrogen, (C₀-C₃ alkyl)NR¹⁰R¹¹, (C₀-C₃ alkyl)OR¹⁰, (C₁-C₃ alkyl)SR¹⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl)3-6-membered heterocyclyl or (C₀-C₃ alkyl)5-6-membered heteroaryl, wherein said alkyl, alkenyl, alkynyl, cycloalkyl, heterocyclyl, heteroaryl and phenyl are optionally substituted by halogen, oxo, OR¹⁶, NR¹⁶R¹⁷ or C₁-C₃ alkyl;
R¹⁰ and R¹¹ are independently hydrogen or C₁-C₆ alkyl optionally substituted by halogen; or
R¹⁰ and R¹¹ are taken together with the atom to which they are attached to form a 3-6 membered heterocyclyl optionally substituted by halogen, oxo or C₁-C₃ alkyl;
R¹² is hydrogen, C₁-C₃ alkyl or halogen;
R¹³ is hydrogen, C₁-C₃ alkyl, C₁-C₃ alkenyl, C₁-C₃ alkynyl or halogen, wherein said alkyl, alkenyl and alkynyl are optionally substituted by OR¹⁸;
R¹⁴ is hydrogen, C₁-C₃ alkyl or halogen;
R¹⁵ is hydrogen or C₁-C₃ alkyl optionally substituted by halogen;
R¹⁶ and R¹⁷ are independently hydrogen or C₁-C₆ alkyl optionally substituted by halogen; or
R¹⁶ and R¹⁷ are taken together with the atom to which they attached to form a 3-6 membered heterocyclyl optionally substituted by halogen, oxo or C₁-C₃ alkyl; and
R¹⁸ is hydrogen or C₁-C₃ alkyl.

45. A compound of claim 1 selected from the group consisting of:
4-Amino-quinazoline-8-carboxylic acid [2,6-difluoro-3-(propane-1-sulfonylamino)-phenyl]-amide;
4-Amino-pyrido[4,3-d]pyrimidine-8-carboxylic acid [2,6-difluoro-3-(propane-1-sulfonylamino)-phenyl]-amide;
4-Amino-quinazoline-8-carboxylic acid [2-chloro-6-fluoro-3-(propane-1-sulfonylamino)-phenyl]-amide;
4-Amino-quinazoline-8-carboxylic acid [6-fluoro-2-methoxy-3-(propane-1-sulfonylamino)-phenyl]-amide;
4-Amino-quinazoline-8-carboxylic acid [2-chloro-3-(propane-1-sulfonylamino)-phenyl]-amide;
4-Amino-quinazoline-8-carboxylic acid [2-fluoro-5-(propane-1-sulfonylamino)-phenyl]-amide;
4-Amino-quinazoline-8-carboxylic acid [2,6-dichloro-3-(propane-1-sulfonylamino)-phenyl]-amide;
4-Amino-quinazoline-8-carboxylic acid [2-fluoro-3-(propane-1-sulfonylamino)-phenyl]-amide;
4-Amino-quinazoline-8-carboxylic acid [2,3,6-trifluoro-5-(propane-1-sulfonylamino)-phenyl]-amide;
4-Amino-quinazoline-8-carboxylic acid [2-chloro-6-fluoro-3-(2-methyl-propane-1-sulfonylamino)-phenyl]-amide;
4-Amino-quinazoline-8-carboxylic acid [2,5-difluoro-3-(propane-1-sulfonylamino)-phenyl]-amide;
4-Amino-quinazoline-8-carboxylic acid [2,6-difluoro-3-(2-methyl-propane-1-sulfonylamino)-phenyl]-amide;
4-Amino-quinazoline-8-carboxylic acid (3-cyclopropylmethanesulfonylamino-2,6-difluoro-phenyl)-amide;
4-Amino-quinazoline-8-carboxylic acid [5-chloro-2-fluoro-3-(propane-1-sulfonylamino)-phenyl]-amide;
4-Amino-quinazoline-8-carboxylic acid [6-chloro-2-fluoro-3-(2-methyl-propane-1-sulfonylamino)-phenyl]-amide;
4-Amino-quinazoline-8-carboxylic acid (2-chloro-3-cyclopropylmethanesulfonylamino-6-fluoro-phenyl)-amide;
4-Amino-quinazoline-8-carboxylic acid [6-chloro-2-fluoro-3-(propane-1-sulfonylamino)-phenyl]-amide;
4-Amino-quinazoline-8-carboxylic acid (6-chloro-3-ethanesulfonylamino-2-fluoro-phenyl)-amide;
4-Amino-quinazoline-8-carboxylic acid (3-ethanesulfonylamino-2,6-difluoro-phenyl)-amide;
4-Amino-quinazoline-8-carboxylic acid (2-chloro-3-ethanesulfonylamino-6-fluoro-phenyl)-amide;
4-Amino-quinazoline-8-carboxylic acid [2-methyl-5-(propane-1-sulfonylamino)-phenyl]-amide;
4-Amino-quinazoline-8-carboxylic acid [2-chloro-5-(propane-1-sulfonylamino)-phenyl]-amide;
4-Amino-quinazoline-8-carboxylic acid [2,6-difluoro-3-(3-fluoro-propane-1-sulfonylamino)-phenyl]-amide;
4-Amino-quinazoline-8-carboxylic acid [2-chloro-6-fluoro-3-(3-fluoro-propane-1-sulfonylamino)-phenyl]-amide;
4-Amino-quinazoline-8-carboxylic acid [6-chloro-2-fluoro-3-(3-fluoro-propane-1-sulfon ylamino)-phenyl]-amide;
4-Amino-quinazoline-8-carboxylic acid [2,3,6-trifluoro-5-(3-fluoro-propane-1-sulfonylamino)-phenyl]-amide;
4-Amino-quinazoline-8-carboxylic acid [2,6-dichloro-3-(3-fluoro-propane-1-sulfonylamino)-phenyl]-amide;
4-Amino-quinazoline-8-carboxylic acid [2-cyano-6-fluoro-3-(propane-1-sulfonylamino)-phenyl]-amide;
4-Amino-quinazoline-8-carboxylic acid [6-chloro-2-cyano-3-(propane-1-sulfonylamino)-phenyl]-amide;
4-Amino-quinazoline-8-carboxylic acid [2-cyano-3-(propane-1-sulfonylamino)-phenyl]-amide;
4-Amino-quinazoline-8-carboxylic acid [6-chloro-2-ethynyl-3-(propane-1-sulfonylamino)-phenyl]-amide;
4-Amino-quinazoline-8-carboxylic acid (3-benzenesulfonylamino-2,6-difluoro-phenyl)-amide;
4-Amino-quinazoline-8-carboxylic acid [2,6-difluoro-3-(furan-2-sulfonylamino)-phenyl]-amide;
4-Amino-quinazoline-8-carboxylic acid [2,6-difluoro-3-(pyrrolidine-1-sulfonylamino)-phenyl]-amide;
4-Amino-quinazoline-8-carboxylic acid [2-cyano-6-fluoro-3-(pyrrolidine-1-sulfonylamino)-phenyl]-amide;
4-Amino-N-(3-(N,N-dimethylsulfamoylamino)-2,6-difluorophenyl)quinazoline-8-carboxamide;
4-Amino-N-(2-chloro-3-(N-ethyl-N-methylsulfamoylamino)-6-fluorophenyl)quinazoline-8-carboxamide;
4-Amino-N-(6-chloro-3-(N,N-dimethylsulfamoylamino)-2-fluorophenyl)quinazoline-8-carboxamide;
4-Amino-N-(2-chloro-3-(N,N-dimethylsulfamoylamino)-6-fluorophenyl)quinazoline-8-carboxamide;
4-Amino-6-(3-hydroxy-prop-1-ynyl)-quinazoline-8-carboxylic acid [2,6-difluoro-3-(propane-1-sulfonylamino)-phenyl]-amide;
4-Cyclohexylamino-quinazoline-8-carboxylic acid [2,3,6-trifluoro-5-(propane-1-sulfonylamino)-phenyl]-amide;
4-(Tetrahydropyran-4-ylamino)-quinazoline-8-carboxylic acid [2,3,6-trifluoro-5-(propane-1-sulfonylamino)-phenyl]-amide;
4-(2-Fluoro-ethylamino)-quinazoline-8-carboxylic acid [2,3,6-trifluoro-5-(propane-1-sulfonylamino)-phenyl]-amide;
4-(Piperidin-4-ylamino)-quinazoline-8-carboxylic acid [2,3,6-trifluoro-5-(propane-1-sulfonylamino)-phenyl]-amide;
4-Cyclopropylamino-quinazoline-8-carboxylic acid [2,3,6-trifluoro-5-(propane-1-sulfonylamino)-phenyl]-amide;
4-(4,4-Difluoro-cyclohexylamino)-quinazoline-8-carboxylic acid [2,3,6-trifluoro-5-(propane-1-sulfonylamino)-phenyl]-amide;
4-(Morpholin-4-ylamino)-quinazoline-8-carboxylic acid [2,3,6-trifluoro-5-(propane-1-sulfonylamino)-phenyl]-amide;
4-(1-Methyl-1H-pyrazol-3-ylamino)-N-(2,3,6-trifluoro-5-(propylsulfonamido)phenyl)quinazoline-8-carboxamide;
4-Ethylamino-quinazoline-8-carboxylic acid [2,3,6-trifluoro-5-(propane-1-sulfonylamino)-phenyl]-amide;
4-Methylamino-quinazoline-8-carboxylic acid [2,3,6-trifluoro-5-(propane-1-sulfonylamino)-phenyl]-amide;
4-(Cyclopropylamino)-N-(2,3,6-trifluoro-5-(3-fluoropropylsulfonamido)phenyl)quinazoline-8-carboxamide;
4-Ethylamino-quinazoline-8-carboxylic acid [2,3,6-trifluoro-5-(3-fluoro-propane-1-sulfonylamino)-phenyl]-amide;
4-Cyclopropylamino-quinazoline-8-carboxylic acid (3-cyclopropylmethanesulfonylamino-2,6-difluoro-phenyl)-amide;
Quinazoline-8-carboxylic acid [2,6-difluoro-3-(propane-1-sulfonylamino)-phenyl]amide
Quinazoline-8-carboxylic acid [2,6-difluoro-3-(3-fluoro-propane-1-sulfonylamino)-phenyl]-amide;
Quinazoline-8-carboxylic acid (3-cyclopropylmethanesulfonylamino-2,6-difluoro-phenyl)-amide;
Quinazoline-8-carboxylic acid [2,6-dichloro-3-(3-fluoro-propane-1-sulfonylamino)-phenyl]-amide;
Quinazoline-8-carboxylic acid [6-chloro-2-fluoro-3-(propane-1-sulfonylamino)-phenyl]-amide;
Quinazoline-8-carboxylic acid [6-chloro-2-fluoro-3-(3-fluoro-propane-1-sulfonylamino)-phenyl]-amide;
4-Methyl-quinazoline-8-carboxylic acid [2,6-dichloro-3-(3-fluoro-propane-1-sulfonylamino)-phenyl]-amide;
4-Methyl-quinazoline-8-carboxylic acid [2,6-difluoro-3-(3-fluoro-propane-1-sulfonylamino)-phenyl]-amide;
4-Methyl-quinazoline-8-carboxylic acid [6-chloro-2-fluoro-3-(3-fluoro-propane-1-sulfonylamino)-phenyl]-amide;
4-Amino-pyrido[3,2-d]pyrimidine-8-carboxylic acid [2-chloro-6-fluoro-3-(propane-1-sulfonylamino)-phenyl]-amide;
4-Amino-pyrido[3,2-d]pyrimidine-8-carboxylic acid [2,6-difluoro-3-(propane-1-sulfonylamino)-phenyl]-amide;
4-Amino-pyrido[3,2-d]pyrimidine-8-carboxylic acid [6-chloro-2-fluoro-3-(propane-1-sulfonylamino)-phenyl]-amide;
4-Amino-pyrido[3,2-d]pyrimidine-8-carboxylic acid [2,6-difluoro-3-(3-fluoro-propane-1-sulfonylamino)-phenyl]-amide;
4-Amino-pyrido[3,2-d]pyrimidine-8-carboxylic acid [6-chloro-2-fluoro-3-(3-fluoro-propane-1-sulfonylamino)-phenyl]-amide
4-Amino-pyrido[3,2-d]pyrimidine-8-carboxylic acid [6-carbamoyl-2-fluoro-3-(3-fluoro-propane-1-sulfonylamino)-phenyl]-amide;
4-Amino-pyrido[3,2-d]pyrimidine-8-carboxylic acid [6-cyano-2-fluoro-3-(propane-1-sulfonylamino)-phenyl]-amide;
4-Amino-6-methyl-quinazoline-8-carboxylic acid [2,6-difluoro-3-(propane-1-sulfonylamino)-phenyl]-amide;
4-Cyclopentylamino-6-methyl-quinazoline-8-carboxylic acid [2,6-difluoro-3-(propane-1-sulfonylamino)-phenyl]-amide;
6-Methyl-4-(1-methyl-azetidin-3-ylamino)-quinazoline-8-carboxylic acid [2,6-difluoro-3-(propane-1-sulfonylamino)-phenyl]-amide;
4-Cyclopropylamino-6-methyl-quinazoline-8-carboxylic acid [2,6-difluoro-3-(propane-1-sulfonylamino)-phenyl]-amide;
6-Methyl-4-(tetrahydro-pyran-4-ylamino)-quinazoline-8-carboxylic acid [2,6-difluoro-3-(propane-1-sulfonylamino)-phenyl]-amide;
4-Cyclobutylamino-6-methyl-quinazoline-8-carboxylic acid [2,6-difluoro-3-(propane-1-sulfonylamino)-phenyl]-amide;
4-(4,4-Difluoro-cyclohexylamino)-6-methyl-quinazoline-8-carboxylic acid [2,6-difluoro-3-(propane-1-sulfonylamino)-phenyl]-amide;
4-Amino-6-methyl-quinazoline-8-carboxylic acid [2-chloro-6-fluoro-3-(propane-1-sulfonylamino)-phenyl]-amide;
4-Amino-6-fluoro-quinazoline-8-carboxylic acid [2-chloro-6-fluoro-3-(propane-1-sulfonylamino)-phenyl]-amide; and
6-Methyl-quinazoline-8-carboxylic acid [2,6-difluoro-3-(propane-1-sulfonylamino)-phenyl]-amide.
